(19) United States
(12) Patent Application Publication
(10) Pub. No.: US 2023/0076063 A1
(43) Pub. Date: Mar. 9, 2023

(54) COMPOSITIONS AND METHODS FOR TREATING OR AMELIORATING A MYCOBACTERIUM TUBERCULOSIS INFECTION

(71) Applicant: San Diego State University (SDSU )Foundation, dba San Diego State University Research Foundation, San Diego, CA (US)

(72) Inventors: Faramarz VALAFAR, San Diego, CA (US); Samuel MODLIN, San Diego, CA (US); Derek CONKLE-GUTIERREZ, San Diego, CA (US)

(21) Appl. No.: 17/787,114

(22) PCT Filed: Dec. 18, 2020
(86) PCT No.: PCT/US2020/066225

§ 371 Date: Jun. 17, 2022

Related U.S. Application Data
(60) Provisional application No. 62/950,890, filed on Dec. 19, 2019.

(51) Int. Cl. C12N 15/113; C12Q 1/689

(52) U.S. Cl. CPC C12N 15/1137; C12Q 1/689; C12N 2310/14; C12N 2310/11; C12N 2310/531; C12N 2310/12; C12N 2310/141; C12Q 1/6869

(57) ABSTRACT

In alternative embodiments, provided are products of manufacture and kits, and methods, for treating or ameliorating a Mycobacterium tuberculosis (TB) or a Mycobacterium africanum infection. In alternative embodiments, provided are products of manufacture and kits, and methods, that comprise or comprise use of DNA methylation inhibitory molecules for treating or ameliorating a Mycobacterium tuberculosis (TB) infection. In alternative embodiments, provided are methods and device for classifying drug-resistance phenotype, or diagnosing Multi-drug resistant Tuberculosis (MDR-TB), eXtensively Drug Resistant phenotype (XDR) tuberculosis, or for clinical decision support. In alternative embodiments, provided are kits for or treating or diagnosing drug resistance of, prognosing, or assisting in clinical decision making for a Mycobacterium tuberculosis (TB) or the Mycobacterium africanum infection.

### RELATED APPLICATIONS

This Patent Convention Treaty (PCT) International Application claims the benefit of priority under 35 U.S.C. § 119(e) of U.S. Provisional Patent Application Ser. No. (USSN) 62/950,890, Dec. 19, 2019. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under grant No. R01AI105185-06 awarded by NIH. The government has certain rights in the invention.

### TECHNICAL FIELD

This invention generally relates to infectious diseases and microbial genomics. In alternative embodiments, provided are products of manufacture and kits, and methods, for treating or ameliorating a Mycobacterium tuberculosis (TB) or a Mycobacterium africanum infection. In alternative embodiments, provided are products of manufacture and kits, and methods, that comprise or comprise use of DNA methylation inhibitory molecules for treating or ameliorating a Mycobacterium tuberculosis (TB) or a Mycobacterium africanum infection.

### BACKGROUND

In 2017, Mycobacterium tuberculosis (M. tuberculosis) killed 1.6 million people globally, the most of any infectious disease, despite significant TB control efforts and the availability of effective TB drugs. Multi-drug resistant Tuberculosis (MDR-TB) threatens control efforts and debilitates patients through a grueling and often ineffective treatment regimen (52% success)¹. M. tuberculosis has a low-mutation rate, and is reported to evolve chiefly through single nucleotide polymorphisms (SNPs)². However, subpopulations of the pathogen consistently persist through chemotherapeutics, eventually developing full antibiotic resistance. It is unclear how such a genetically static organism adapts so rapidly to drug treatment and varied immune pressures.

DNA methylation is a plausible yet scarcely explored alternative mechanism for phenotypic variation in M. tuberculosis. M. tuberculosis encodes three known DNA methyltransferases (MTases), MamA, MamB, and HsdM, which each target a different sequence motif for modification. Previous studies have shown that loss-of-function (knockout) variants in these genes are common, and often associate with lineage^{3,4}. These minor differences in genotype result in radically different methylomes, potentially explaining the phenotypic variation observed between lineages⁴. However, these studies examined only a handful of isolates from each lineage of M. tuberculosis, and could therefore not resolve whether MTase activity profiles are lineage-specific.

DNA methylation regulates gene expression in many prokaryotes^{5,6}, including M. tuberculosis. Regulatory interaction between transcription factors and DNA methylation has been mechanistically characterized in several species⁸⁻¹⁰ and hypothesized to occur at a limited number of sites in M. tuberculosis^{3,4}. Cis-regulation by DNA methylation was previously interrogated in M. tuberculosis through Single Molecule Real Time (SMRT)-sequencing, identifying seven differentially methylated sites upstream of differentially expressed genes¹¹. However, the study only examined Euro-American and Indo-Oceanic isolates for this analysis, and only considered the 200 base pairs (bp) upstream of differentially expressed genes.

Heterogeneous DNA methylation has been reported in several bacterial species. Heterogeneous methylation is caused by spontaneous knockout mutations in MTase coding genes¹², site-specific occlusion from DNA binding proteins^{10,13,14}, or intracellular stochastic methylation¹⁵. As DNA methylation regulates gene expression, heterogeneous methylation can create multiple phenotypes within isogenic populations¹². This phenotypic plasticity aids rapid adaptation to changing environmental pressures and nutrient constraints. However, no study has examined heterogeneous methylation in M. tuberculosis.

### SUMMARY

In alternative embodiments, provided are methods for treating or ameliorating a Mycobacterium tuberculosis (TB) or a Mycobacterium africanum infection, comprising inhibiting DNA methylation in an infecting Mycobacterium tuberculosis or a Mycobacterium africanum bacterium or bacterial population, the method comprising administering to an individual in need thereof a DNA methylation inhibitory molecule capable of inhibiting a Mycobacterium tuberculosis or a Mycobacterium africanum DNA methyltransferase,

wherein optionally the DNA methylation inhibitory molecule is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally,

and optionally the DNA methylation inhibitory molecule or the formulation or pharmaceutical composition is contained in or carried in a nanoparticle, a particle, a micelle or a liposome or lipoplex, a polymersome, a polyplex or a dendrimer,

and optionally the DNA methylation inhibitory molecule, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant.

and optionally the DNA methylation inhibitory molecule is an inhibitory nucleic acid, the optionally the inhibitory nucleic acid is contained in a nucleic acid construct or a chimeric or a recombinant nucleic acid, or an expression cassette, vector, plasmid, phagemid or artificial chromosome, optionally stably integrated into a TB cell's chromosome, or optionally stably episomally expressed in a TB cell,

and optionally the inhibitory nucleic acid is or comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme,

and optionally DNA methylation inhibitory molecules for inhibiting MamC (also called HsdM) comprise a small inhibitory molecule comprising:

| siRNA oligo | GC% | SEQ ID NO: 22 |
|---|---|---|
| CACGGCTCAACCGCCGGCTTCCTTA | 64.0 | SEQ ID NO: 23 |
| GCGCGACCTTCAAGAACCGTCTCTT | 56.01 | SEQ ID NO: 24 |
| GGCTCCGACATATTGTTCCTGTTGT | 48.0 | SEQ ID NO: 25 |
| GCTCCGACATATTGTTCCTGTTGTA | 44.0 | SEQ ID NO: 26 |
| CCGAGCCACTCGACTAGCTGGATAA | 56.01 | SEQ ID NO: 27 |
| CAGATGCTTATGAAGGAGCCGTTAA | 44.0 | SEQ ID NO: 28 |
| AGATGCTTATGAAGGAGCCGTTAAA | 40.0 | SEQ ID NO: 29 |
| GCGAAGCCACAAGGCGGGCGGTTAT | 64.0 | SEQ ID NO: 30 |
| TCGCCCGCGACTGGTTGCTCCTCTA | 64.0 | SEQ ID NO: 31 |
| TGCCTCTCGGCTAGCTGCTCTTCTA | 56.01 | SEQ ID NO: 32 |
| TCGGTGAGCTGATCGACCTATTTAA | 44.0 | SEQ ID NO: 33 |
| GATCTGATGGGTGAGGTCTACGAAT | 48.0 | SEQ ID NO: 34 |
| GAGGTCTACGAATACTTCCTCGGCA | 52.0 | SEQ ID NO: 35 |
| GAGGCATGTTTGTGCAGACCGAGAA | 52.0 | SEQ ID NO: 36 |
| GATCCGAAGGATGTCTCGATCTATG | 48.0 | SEQ ID NO: 37 |
| CAGATCGTGGAGGCGGATTTGGTTT | 52.0 | SEQ ID NO: 38 |
| CGGCTGCCGTCAAAGGGATTATGTA | 52.0 | SEQ ID NO: 39 |
| GGGTCGCTGTCGGCCAGCCAATACA | 64.0 | SEQ ID NO: 40 |
| CGGCGAAGAACATCGGTCAGCTGAT | 56.01 | SEQ ID NO: 41 |
| CCAGCGTGGTCAAGGTGATCGTGGA | 60.0 | SEQ ID NO: 42 |
| GCAGATCGTGGAGGCGGATTTGGTT | 56.01 | SEQ ID NO: 43 |
| CGCCAAAGACAAGGCGGCAGGTAAG | 60.0 | SEQ ID NO: 44 |
| CGGGTCGAAGTCGGCTGCCGTCAAA | 64.0 | SEQ ID NO: 45 |

and optionally DNA methylation inhibitory molecules for inhibiting MamA comprise a small inhibitory molecule comprising:

| siRNA oligo | GC% | |
|---|---|---|
| CCGCTTCGGAAACTGGGCATCCGAA | 60.0 | SEQ ID NO: 46 |
| CGCTATCGGGAGATCACCCTGGTTA | 56.01 | SEQ ID NO: 47 |
| TGGTTACCTTCGAGCGGCTGGTGTT | 56.01 | SEQ ID NO: 48 |
| GCCGACGTGGATGTGGGCATCGTGA | 64.0 | SEQ ID NO: 49 |
| GCGCCCAACTCAGCGGGCTGATCTA | 64.0 | SEQ ID NO: 50 |
| CCGACCTCTTTATGCTGCGCCAGAT | 56.01 | SEQ ID NO: 51 |
| CCCGAACGTCGATCCGGCAACTCTT | 60.0 | SEQ ID NO: 52 |
| CATCTTGGAGTTGGAGCCTAGGGAA | 52.0 | SEQ ID NO: 53 |
| GCGCAGAACTTGCCCAGGATGTTGA | 56.01 | SEQ ID NO: 54 |
| CCCAGGATGTTGATCTCCTGCTGAA | 52.0 | SEQ ID NO: 55 |
| CGCACACCCGGATGCTGGCTGGTTT | 64.0 | SEQ ID NO: 56 |
| CACCCGGATGCTGGCTGGTTTGACT | 60.0 | SEQ ID NO: 57 |
| GGATGCTGGCTGGTTTGACTGGTTA | 52.0 | SEQ ID NO: 58 |
| CCGAGCCGCTACGCTTGCTAGACTT | 60.0 | SEQ ID NO: 59 |
| GCTCCTGAGTTTGTCAGGCGGTGAT | 56.01 | SEQ ID NO: 60 |
| CGAAGAAGTGCAAGTGGCTACGGTT | 52.0 | SEQ ID NO: 61 |
| CCACAAGGTGTTGTCGCGCTGTAAG | 56.01 | SEQ ID NO: 62 |
| GCTGTAAGCGCAAGCGGCTCTAGTA | 56.01 | SEQ ID NO: 63 |
| GCGGCTCTAGTACCCGGCGTCAATA | 60.0 | SEQ ID NO: 64 |
| CCTCGCGTCTTGAACGGGTCCTACA | 60.0 | SEQ ID NO: 65 |

and optionally DNA methylation inhibitory molecules for inhibiting MamB comprise a small inhibitory molecule comprising:

| siRNA oligo | GC% | |
|---|---|---|
| CCGAAGTGGTTGGCCCACTAGTAGA | 56.01 | SEQ ID NO: 66 |
| CCCAGCTGCTCAAGCTGAACCACTA | 56.01 | SEQ ID NO: 67 |
| GAGGTTCTAGCAGCCGACGACCTTA | 56.01 | SEQ ID NO: 68 |
| GCAGACGGCGCAACCGGCTGTTGTT | 64.0 | SEQ ID NO: 69 |
| CGCTGTTCGACAACCCGCCAGTGTA | 60.0 | SEQ ID NO: 70 |
| CCGACTGGCTGCTCCCGCACGTATA | 64.0 | SEQ ID NO: 71 |
| CACTGTGCACAGAAGCGCTTGATAA | 48.0 | SEQ ID NO: 72 |
| CGCCGAGCCGTGCATGGCTGGTAAA | 64.0 | SEQ ID NO: 73 |
| CGCTGGTTCAGTGGTTTCTGCTGTA | 52.0 | SEQ ID NO: 74 |
| CGGCCTTTGATCGGCTGAATGTACA | 52.0 | SEQ ID NO: 75 |
| GGGTGCACGACGGTCAGTATCTGAA | 56.01 | SEQ ID NO: 76 |
| CCGGTGAAGGCAGCGACAAGCTGTT | 60.0 | SEQ ID NO: 77 |
| GAGCAGTTGGCGATGTTCTCGTTGT | 52.0 | SEQ ID NO: 78 |
| CCAACGAGATCATGCTGCTGGCGTA | 56.01 | SEQ ID NO: 79 |
| GACAACGGTGTTGTCGGATTCGTCT | 52.0 | SEQ ID NO: 80 |
| GCGTGGCCGGTTATCGGCGACAAGA | 64.0 | SEQ ID NO: 81 |
| CCGGGATGCGTGGTGTTACAACTTT | 52.0 | SEQ ID NO: 82 |
| AGCCCTTCTCGTGTCTGATGCTAAA | 48.0 | SEQ ID NO: 83 |
| CAAGTCACCAAAGACGACATCTTCT | 44.0 | SEQ ID NO: 84 |
| CAAGACCACTCAACGATCATCTACA | 44.0 | SEQ ID NO: 85 |

and alternative embodiments the siRNA inhibitory molecule comprises a sequence having at least about 90%, 95%, 98% or more sequence identity to any of these exemplary siRNA sequences.

In alternative embodiments of methods as provided herein:
the Mycobacterium tuberculosis or the Mycobacterium africanum DNA methyltransferase is a methyltransferase selected from the group consisting of MamA, MamB and HsdM; and/or
the DNA methylation inhibitory molecule capable of inhibiting a Mycobacterium tuberculosis DNA methyltransferase is or comprises a small molecule, an inhibitory nucleic acid (optionally and miRNA or antisense molecule), polypeptide or peptide (optionally an antibody capable of specifically binding to the Mycobacterium tuberculosis DNA methyltransferase and inhibiting its expression or activity, a lipid or a polysaccharide.

In alternative embodiments, provided are kits for or treating or ameliorating a tuberculosis (TB) infection, wherein optionally Mycobacterium tuberculosis (TB) or Mycobacterium africanum is the microbacterial agent of infection, comprising a DNA methylation inhibitory molecule capable of inhibiting a Mycobacterium tuberculosis or Mycobacterium africanum DNA methyltransferase, wherein optionally the DNA methylation inhibitory molecule is or comprises a DNA methylation inhibitory molecule used to practice a method as provided herein, and optionally the kit further comprises instructions for practicing a method as provided herein.

In alternative embodiments, provided are methods for treating or ameliorating a tuberculosis (TB) infection, wherein optionally Mycobacterium tuberculosis (TB) or Mycobacterium africanum is the microbacterial agent of infection, comprising inhibiting expression of at least one gene as set forth in Table 1 (FIG. 8), Table 2 (FIG. 9), the “TSS” column of FIG. 20, and/or in the first column (labeled TSS) of FIG. 23, the method comprising administering to an individual in need thereof a molecule capable of inhibiting expression of the gene or a polypeptide encoded by the gene, wherein optionally the molecule capable of inhibiting expression of the gene or a polypeptide encoded by the gene is or comprises a small molecule, an inhibitory nucleic acid (optionally and miRNA or antisense molecule), polypeptide or peptide (optionally an antibody capable of specifically binding to the Mycobacterium tuberculosis or the Mycobacterium africanum DNA methyltransferase and inhibiting its expression or activity, a lipid or a polysaccharide.

In alternative embodiments, provided are kits for or treating or ameliorating a Mycobacterium tuberculosis (TB) or a Mycobacterium africanum infection, comprising a molecule capable of inhibiting expression of at least one gene as set forth in Table 1 (FIG. 8), Table 2 (FIG. 9), the “TSS” column of FIG. 20, and/or in the first column (labeled TSS) of FIG. 23, and optionally further comprising instructions for practicing a method as provided herein.

In alternative embodiments, provided are methods for identifying targets for treating, ameliorating, diagnosing, or prognosing infection by a microbial agent, the method comprising an analysis of single-molecule sequencing data, wherein the analysis comprises deducing knowledge of a DNA sequence and the boundaries of genetic elements encoded therein and deducing knowledge of the base modification status of bases comprising the deduced DNA sequence.

In alternative embodiments of methods as provided herein:
the method provides evidence of druggability and/or utility to a user for helping to clear microbial infection, the method further comprising a series of single-molecule sequencing data processing steps that incorporate signals of DNA sequence order and DNA sequence modification, such that their coincidence is inferred and coincidences between base modification and identified genetic elements of the sequence that evidence druggability and/or utility for helping to clear microbial infection are returned to the user;
the genetic elements encoding a plurality of base modifying enzymes are deduced and/or prior knowledge of the identity of a plurality of genetic elements encoding base modifying enzymes are collated and correlated to sequencing kinetics of sequence contexts that are known/deduced to methylate, in order to deduce of the presence or absence of the phenomenon of intercellular mosaic methylation in the analyzed sample; and/or
the single-molecule sequencing data is processed through a series of analyses and returns estimates of the likelihood of prognostic outcomes based on the presence, absence, or contingencies dictating the presence/absence of the phenomenon of intercellular mosaic methylation to the user of the embodiment.

In alternative embodiments, provided are methods for diagnosing drug resistance in, prognosing, or assisting clinical decision making regarding infections where a Mycobacterium tuberculosis or a Mycobacterium africanum is known as or suspected to be the etiological agent of infection, the method comprising assaying a sample isolated from an infected patient known or suspected to harbor Mycobacterium tuberculosis for the presence of a DNA methylation at locations in a DNA sequence (e.g., a genome), and/or the presence of particular oligonucleotides indicative of capacity for DNA methylation and the degree and presence or capability for intercellular mosaic methylation within the strain(s) of M. tuberculosis or M. africanum infecting the patient.

In alternative embodiments of methods as provided herein:
the Mycobacterium tuberculosis or the Mycobacterium africanum DNA methyltransferase is a methyltransferase selected from the group consisting of MamA, MamB and HsdM;
the DNA methylation assay is or comprises a small reporter and/or bait molecule (optionally an antisense molecule or fluorescent reporter), polypeptide or peptide (optionally an antibody) capable of specifically binding to a sequence within a genetic element encoding Mycobacterium tuberculosis or the Mycobacterium africanum DNA methyltransferase and indicating its presence, absence, or relative abundance; and/or the DNA methylation assay is or comprises a small reporter and/or bait molecule (optionally an antisense molecule or fluorescent reporter), polypeptide or peptide (optionally an antibody) capable of specifically binding to a sequence of interest with differential affinity depending on the methylation status of a base or set of bases within the DNA sequence indicating the presence, absence, or relative abundance of methylation at the locus or set of loci of interest.

In alternative embodiments, provided are kits for or treating or diagnosing drug resistance of, prognosing, or assisting in clinical decision making for a Mycobacterium tuberculosis (TB) or the Mycobacterium africanum infection, comprising a DNA methylation detection assay capable of detecting and quantifying specific DNA sequences encoding DNA methyltransferases, and the DNA methylation status of at least one loci within and specific DNA sequences, wherein the at least one loci comprises a gene as set forth in the first column (labeled TSS) of FIG. 23, and/or FIG. 21, and optionally further instructions for practicing a method as provided herein.

In alternative embodiments, provided are methods of identifying a set of DNA base modification sites with discriminatory attributes in a bacterial species, based on the variability in modified fraction at the locus across samples of the bacterial species with the modifying enzyme active, as determined by a method as provided herein. In alternative embodiments, provided are methods of identifying and quantifying heterogeneity within a bacterial sample wherein the modification status of sites derived using the method as provided herein are used to identify and quantify heterogeneity and the phases that comprise it. In alternative embodiments, the base modification status at base modification sites is quantified by a reporter of modification status. In alternative embodiments, the length of DNA sequence fragments following exposure to an enzyme with catalytic specificity for unmodified DNA of the same sequence or a sequence overlapping the modification motif of interest is used as the reporter of modification status, and the method comprises:
a. extracting DNA from the bacterial sample of interest,
b. bathing the extracted DNA in a solution containing a DNA degradatory enzyme, an enzyme with a catalytic specificity for a DNA sequence motif, or a DNA endonuclease,
c. post-bathing, optionally amplifying the digested DNA and performing gel electrophoresis to separate digested DNA fragments according to their mass, charge, and optionally other physical attributes,
d. calibrating the length travelled in gel to DNA length in empirical control experiments on DNA of known length and nucleotide content,
e. mapping the lengths of fragmented DNA back to the modification motif sites in a de novo assembled genome to identify what two modification sites intervened by each DNA fragment;
f. quantifying the DNA fragments and using information obtained from (e.) to quantify modification patterns within the sample assayed.

In alternative embodiments, provided are methods for identifying modification sites with utility for classifying a bacterial phenotype of interest from the discriminatory modification sites identified through a method as provided herein.

In alternative embodiments, provided are methods for identifying pairs of modification sites as identified using methods as provided herein, that are additively informative for classifying a bacterial phenotype of interest.

In alternative embodiments, provided are methods for identifying pairs of modification sites identified through the methods as provided herein, for designing reporters of modification status by DNA fragment length. In alternative embodiments, the degradatory enzyme is a restriction endonuclease; the base modification of interest is DNA methylation and the modifying enzyme(s) are DNA methyltransferases; the bacterial species of interest is a human pathogen; the human pathogen is a member of the Mycobacterium tuberculosis or the Mycobacterium africanum complex; the member of the Mycobacterium tuberculosis complex is Mycobacterium tuberculosis or the Mycobacterium africanum; the member of the Mycobacterium tuberculosis complex is Mycobacterium africanum; the DNA methyltransferases are MamA, MamB, and HsdM; the discriminatory DNA methylation sites are those in FIG. 21; and/or, the method comprises use of a device for classifying drug-resistance phenotype, or diagnosing Multi-drug resistant Tuberculosis (MDR-TB, or MDR), eXtensively Drug Resistant phenotype (XDR) tuberculosis, or for clinical decision support.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A schematically illustrates that the methylomes of a global collection of 93 clinical isolates from all seven lineages of the M. tuberculosis complex (MTBC) were analyzed and the sequence of each isolate was de novo assembled into complete, circularized genomes and integrated with gene, promoter, and transcription factor binding site data;

FIG. 1B schematically illustrates how the analyzed genomes was Single Molecule Real Time (SMRT) sequenced and kinetic data was processed;

FIG. 1C schematically illustrates the relationship of DNA methyltransferase (MTase) genotype, epigenotype, phylogenetics and gene heterogeneity;

FIG. 1D schematically illustrates how this data was subjected to a methylome survey comparing motif sites with isolates, and promoter methylation, including showing the nucleic acid motif (SEQ ID NO:1) TANNNT; and nucleic acid sequences: (SEQ ID NO:2) TGGAATATTCTGGAGTCATGTCAGAGA; and (SEQ ID NO:3) ACCTTATAAGACCTCAGTACAGTCTCT; and

FIG. 1E schematically illustrates how using Bayes's classifier consistently hypomethylated loci found, where the sequence (SEQ ID NO:4) is repeated 3 times: CCCACCTGGAGAGTATCGCTGGAGATGTCGACACGCAGGCTGT.

FIG. 2A-D illustrate MTase activity patterns and genotypes across clinical and reference strains:

FIG. 2A illustrate images of boxplot of IPD ratio distributions within MamA (top pane), HsdM (middle pane), and MamB (bottom pane) target motifs for each M. tuberculosis isolate, where the boxplots are colored by mamA, hsdM, and mamB genotype;

FIG. 2B illustrates images of SNP-based phylogenetic trees with mutations mapped for each MTase;

FIG. 2C illustrates images of phylogeny of isolates with branches colored according to the MTase activity profile, where colors of the outer rung indicate lineage;

FIG. 2D graphically illustrates images density traces of sequencing kinetics for each isolate at every motif site, organized into panes by MTase (columns) and lineage (rows), and colored by the activity of their MTase;

as described in further detail in Example 1, below.

FIG. 3 graphically illustrates SNP distance versus differential methylation, showing a dot plot comparing the number of differentially methylated sites to SNP distance between clinical isolates and virulent M. tuberculosis type strain H37Rv;

as described in further detail in Example 1, below.

FIG. 4A-F illustrate characterizing methylation heterogeneity through SMALR, where native score (nat) is the subread-normalized natural log of IPDs:

FIG. 4A-D graphically illustrate the distribution of native scores among subreads for each isolate of the specified genotype, where each colored trace represents a single isolate;

FIG. 4A shows has a light blue trace with a mean native score identical to that of W136R;

FIG. 4B shows a light violet trace with a mean native score identical to that of the wild-type;

FIG. 4C (E270A genotype) and FIG. 4D (G152S genotype) both show reference traces, where each reference trace shares the same number of measurements (isolates) and identical standard deviation as the genotype it is being compared to;

FIG. 4E graphically illustrates the effect of methionine starvation inducing heterogeneous methylation within single subreads;

FIG. 4F graphically illustrates the kinetics of Methionine-starved H37Rv_{ΔmetA} differ from phase variant simulated mixture; and

FIG. 4G schematically illustrates stochastic versus phase-variable methylation, and how phase-variable methylation leads to discrete phases and stochastic phase-variable methylation leads to mosaic methylation;

as described in further detail in Example 1, below.

FIG. 5A-E illustrate that common motif sites were surveyed to observe how they varied across isolates and find common attributes among sets of sites following in vitro culture;

FIG. 5A illustrates that across isolates of the same genotype, IPD Ratio distributions were consistent within sets of isolates with the same genotype, except for knockdown mutants, highlighting these mutants as pertinent targets for further inquiry, and that most of the interesting isolates harbored knockdown mutations in mamA, although a few wild-type hsdM isolates exhibited methylation patterns inconsistent with the rest;

FIG. 5B illustrates density of common motif sites in E270A mutants, knockout mutants and wild type; and

FIG. 5C illustrates images of variability among active isolates in hsdM isolates (upper image), MamA isolates (middle image) and MamB isolates (lower image);

as described in further detail in Example 1, below.

FIG. 6A graphically illustrates that the IPD ratio at cobK:304 across HsdM active isolates was bimodal; and

FIG. 6B illustrates that the 18 cobK:304 methylated isolates were all Indo-Oceanic, and grouped together in our phylogenetic tree;

as described in further detail in Example 1, below.

FIG. 7A-E illustrate the configuration of orphan MTase motif sites at promoters:

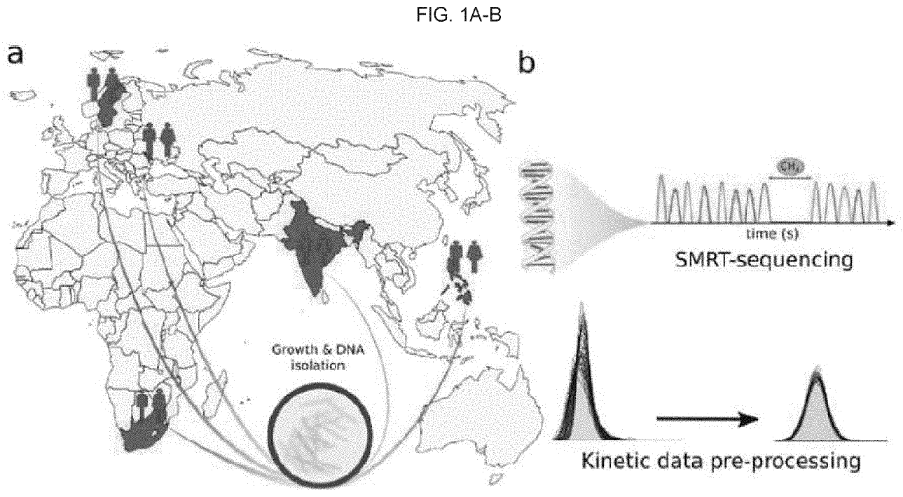

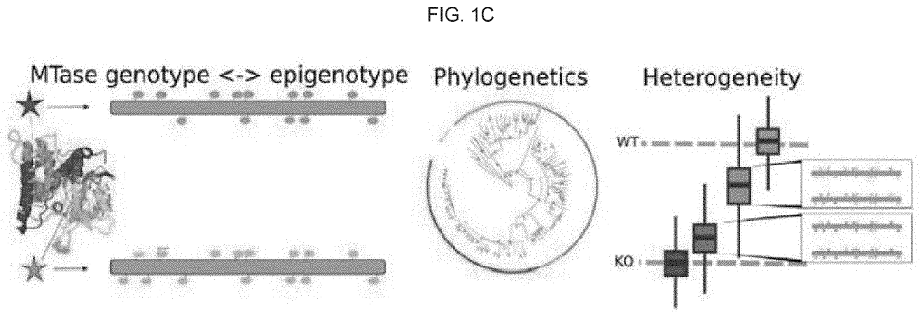

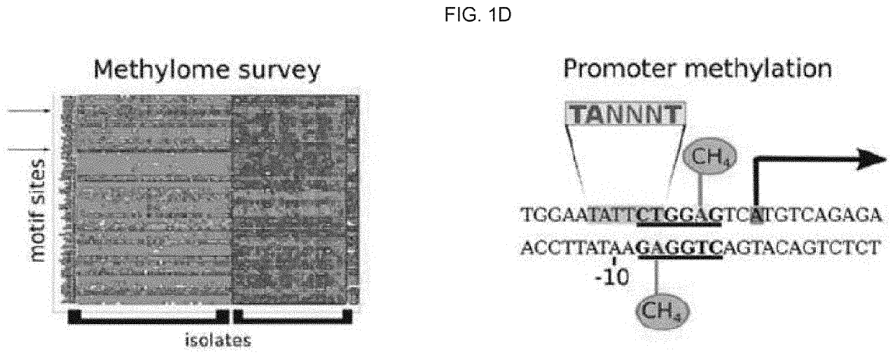

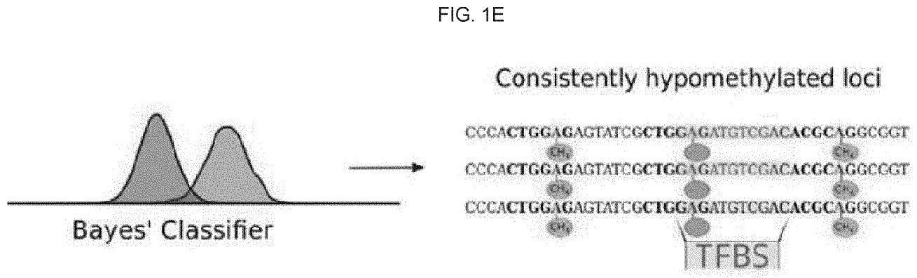

FIG. 1A graphically illustrates an MTase-SFBS-promoter configuration where for each MTase, a frequency plot is displayed for occurrences of unique MTase motifs at distances upstream of the TSS, and the canonical SigA binding motif is superimposed for conceptual clarity, but other SFBSs, and loci with no known SFBS in the −7 to −12 bp window upstream of annotated TSS are also included, and the sigma factor binding nucleic acid motif TANNNT (SEQ ID NO:1) is shown; also showing the peptide sequences GATYNNNNRTAC (SEQ ID NO: 5), CTGGAG (SEQ ID NO:6), GTANNNNATC (SEQ ID NO:7);

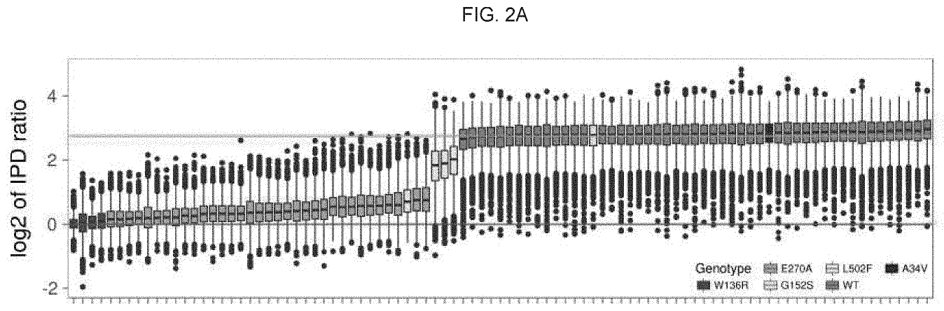

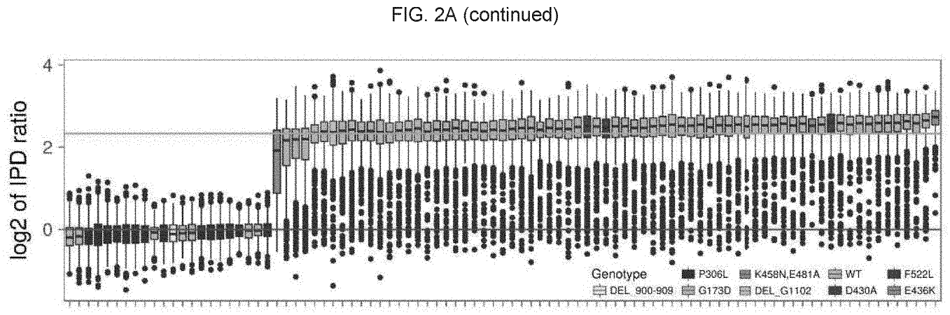

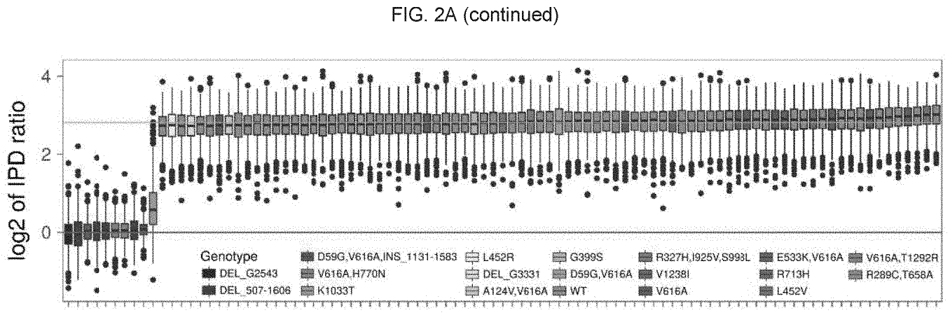

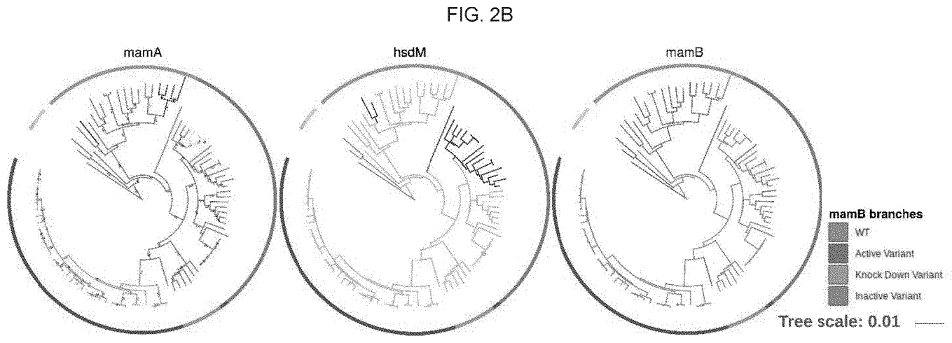

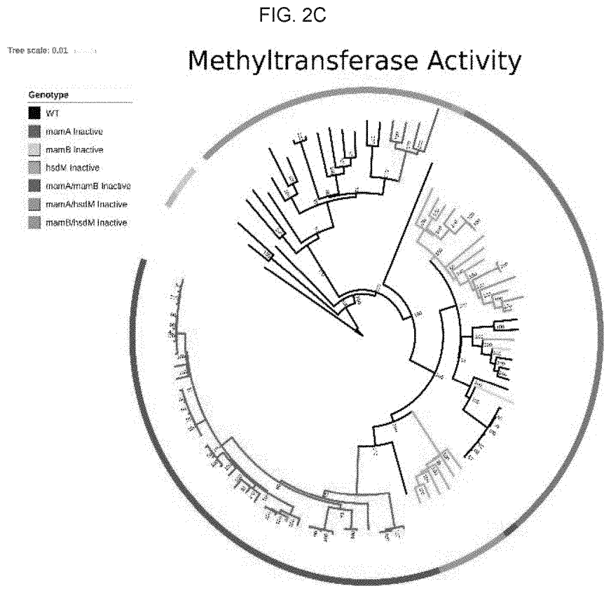

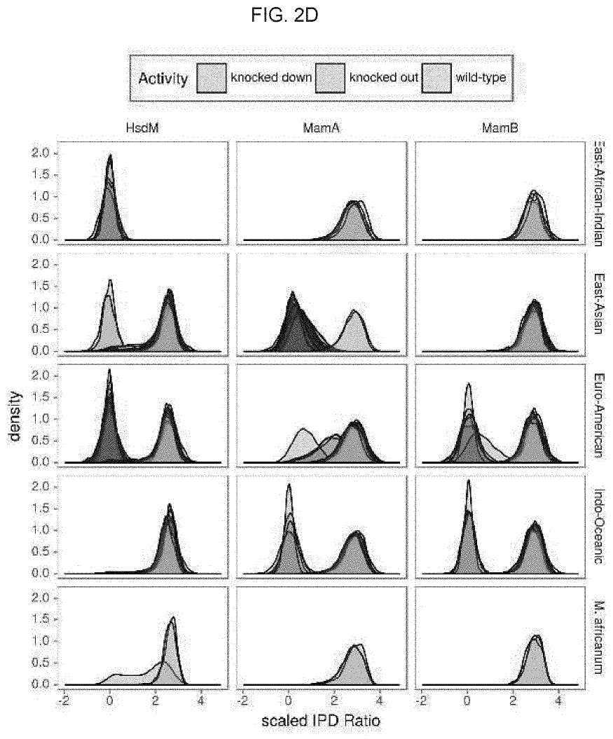

FIG. 2B graphically illustrates a histogram of the number of promoters with the −10 element overlapping a MTase motif site in at least 30 isolates, for each MTase and sigma factor;

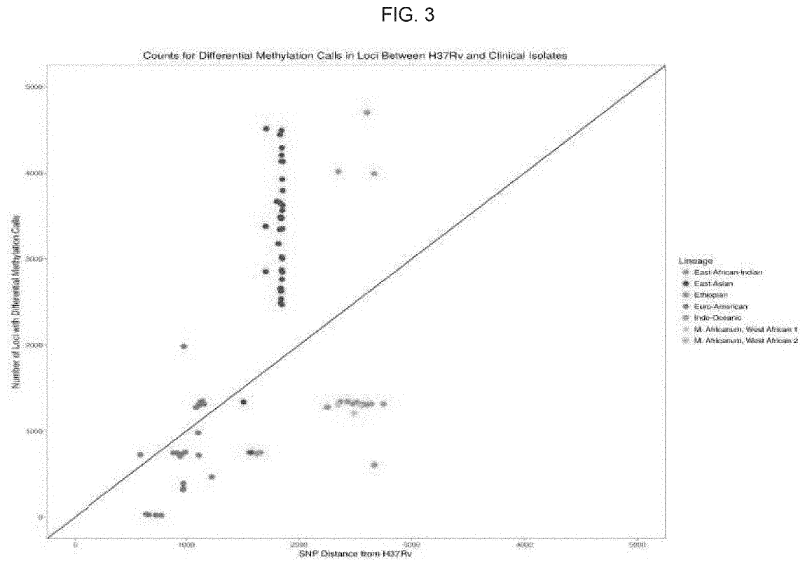

FIG. 3C graphically illustrates variability (SD of log 2(IPD Ratio) across isolates) in sequencing kinetics across isolates with active MTase (y-axis) for common promoter motifs positioned according to their distance upstream of their TSS (x-axis), where motif sites within three SD of the mean for MamB motifs are grey, and the outliers are highlighted in red, and labelled with downstream gene;

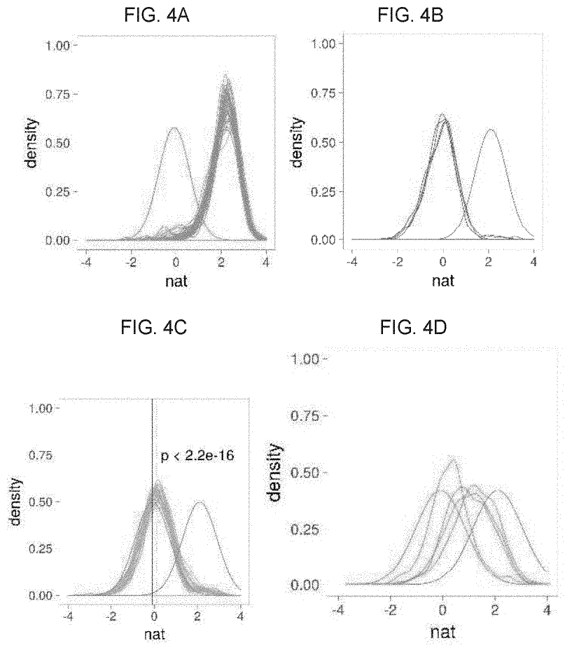

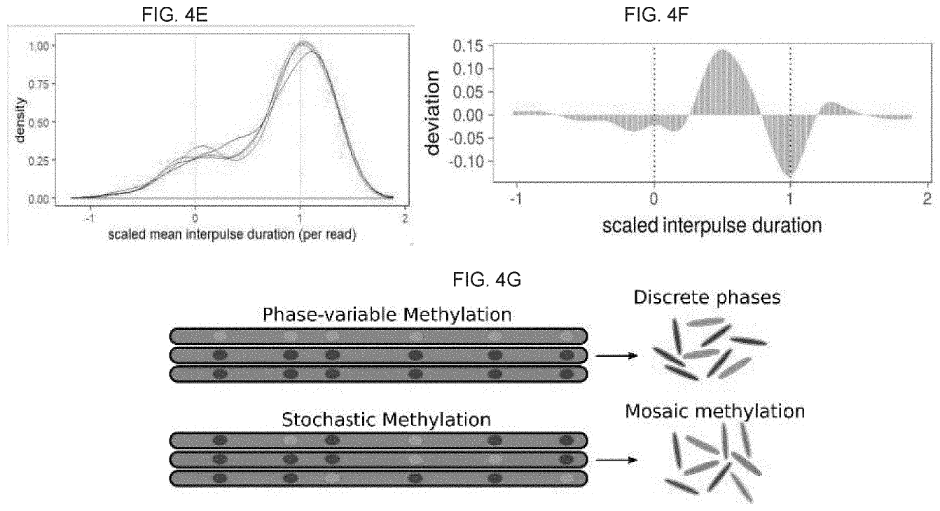

FIG. 4D graphically illustrates images of stacked histograms of a number of genes harboring promoter motif sites for each MTase, where darker shades indicate progressively substantiated promoters; and

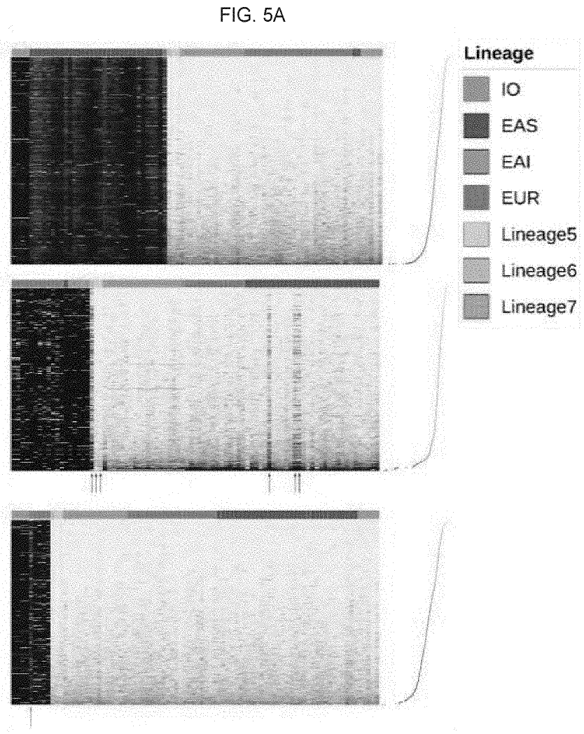

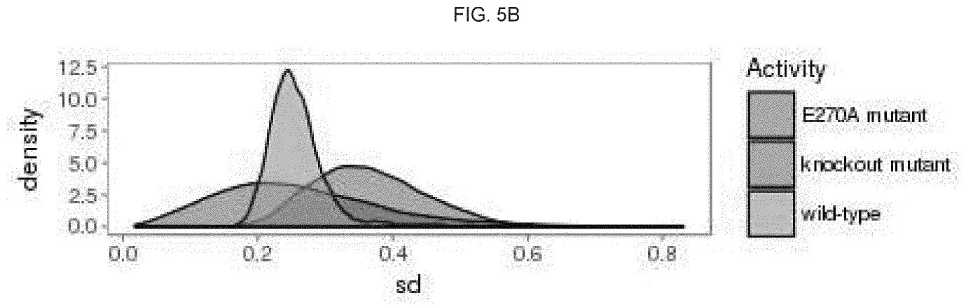

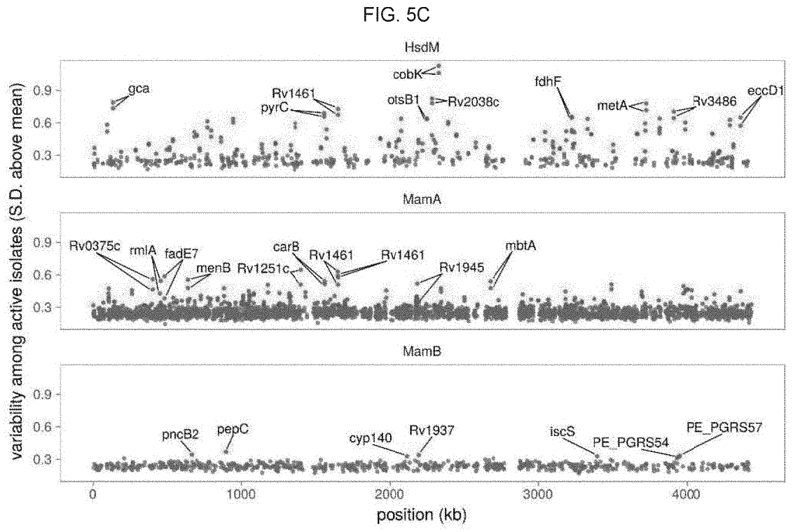

FIG. 5E graphically illustrates differentially expressed genes in a ΔHsdM study where all HsdM promoter motifs are positioned according to position within the promoter and Benjamin-Hochberg adjusted −log 10(p-value);

as described in further detail in Example 1, below.

FIG. 8 illustrates Table 1 which shows consistently hypomethylated MTase motif sites across clinical M. tuberculosis isolates; as described in further detail in Example 1, below.

FIG. 9 illustrates Table 2 which shows that promoter MTase motifs and hypomethylated motifs are in genes required to acquire host lipids, dictate their metabolic fate, and detoxify intermediates generated during their utilization; as described in further detail in Example 1, below.

FIG. 10 illustrates a DNA methylation inhibitory molecule used in exemplary methods as provided herein as described in Yadav M K, et al (2015), PLoS ONE 10(10): e0139238.

FIG. 11A-B illustrate images of heatmaps showing the activity of isolates, as indicated at the bottom of each heatmap, where isolates within each heatmap are sorted first by activity, and then by lineage, and lineage is shown at the top of each heatmap:

FIG. 11A illustrates a heatmap for MamA motifs; and

FIG. 11B illustrates a heatmap similar to the heatmap in FIG. 11A, but for HsdM motif sites, wherein all common motif sites within 50 bp upstream of a TSS are shown;

as described in further detail in Example 1, below.

FIG. 12A-C illustrate real sequencing kinetics and resistance phenotype data from Mycobacterium tuberculosis and Mycobacterium africanum, demonstrating proof-of-concept for this particular application, and exemplifying the general method; and FIG.D-F illustrate conceptual depictions of the remaining steps comprising the diagnostic/clinical decision support tool:

FIG. 12A graphically illustrates the identification of hypervariable motif sites;

FIG. 12B graphically illustrates associating modification status with phenotype and a Manhattan plot of the significance of association between methylated fraction of hypervariable motif sites identified in FIG. 12A and resistance phenotypes for 7 common anti-TB drugs, and eXtensively Drug Resistant phenotype (XDR);

FIG. 12C illustrates identifying correlated and uncorrelated sites;

FIG. 12D illustrates cutting DNA at unmodified motifs GAATTC (SEQ ID NO:8) and CTTAAG (SEQ ID NO:9);

FIG. 12E illustrates an image quantitating DNA fragment lengths using gel electrophoresis; and

FIG. 12F graphically illustrates readout modification abundances and classifications;

as described in further detail in Example 1, below.

FIG. 13A-C illustrate MamB mutations mapped to annotated functional domains and predicted 3D structure, where mapping of mutations in mamB, and their effect on methyltransferase (MTase) function, at (FIG. 13A) primary (multiple sequence alignment with mutations superimposed) (M. bovis active (SEQ ID NO: 10), M. microti active (SEQ ID NO:11), EAS-1 active (SEQ ID NO:12), EAS-3 active (SEQ ID NO:13), EAS-2 active (SEQ ID NO:14), 10-1 active (SEQ ID NO:15), 10-3 active (SEQ ID NO:16), 10-2 active (SEQ ID NO:17), EAM-1 active (SEQ ID NO:18), EAM-3 inactive (SEQ ID NO:19), and EAM-2 inactive (SEQ ID NO:20)), (FIG. 13B) secondary (mapped to functional domain annotation), and (FIG. 13C) tertiary levels (mapped to putative structures) of abstraction; as described in further detail in Example 1, below.

FIG. 14A-E graphically illustrate preprocessing quality control:

FIG. 14A graphically illustrates distribution of inter pulse duration (IPD) ratios across all bases for one of the replicate runs of H37Ra;

FIG. 14B graphically illustrates log 2 transformation converts log-normal distribution into normal distribution of log 2(IPD ratios), expressed in standard deviations from the mean (sd);

FIG. 14C graphically illustrates difference in sequencing kinetics between replicate H37Ra SMRT-sequencing runs across the genome;

FIG. 14D graphically illustrates difference in log 2(IPD ratio) between replicate runs as a function of coverage; and

FIG. 14E graphically illustrates Quantile-Quantile plot comparing IPD ratios at a subset of mamA motif (blue) and at non-mamA motifs (red) to theoretical values in a perfect normal distribution (black diagonal line), and also shows (SEQ ID NO:6) CTGGAG);

as described in further detail in Example 1, below.

FIG. 15A-C graphically illustrate methylation heterogeneity found in mamB:K1033T allele, where all three graphs measure native scores for all isolates of a genotype:

FIG. 15A graphically illustrates the wildtype, and the mean native score is 2.19, similar to that of the mamA wildtype isolates;

FIG. 15B graphically illustrates the K1033T genotype with only one isolate, and this graph possesses a dotted vertical line identifying the mean native score and a solid black line that identifies the mean native score of the inactive genotypes;

FIG. 15C graphically illustrates a representation of all mamB-knockout genotypes;

as described in further detail in Example 1, below.

FIG. 16A-C graphically illustrate evaluation of Bayesian Classifier for MamA (FIG. 16A), HsdM (FIG. 16B), and MamB (FIG. 16C):

FIG. 16D illustrates a histogram reporting the distribution of IPD ratios among bases within the target motifs of known methyltransferases HsdM, MamA, and MamB, after normalizing the IPD ratios of each base to the mean IPD ratio of all adenines within the isolate, and log transforming the data; and

FIG. 16E illustrates a violin plot showing the distribution of coverage at MTase motif sites, aggregated from all clinical isolates;

as described in further detail in Example 1, below.

FIG. 17A-C illustrate analyses of hypomethylated and hypervariable motif sites:

FIG. 17A graphically illustrates distribution of normalized IPD ratios at consistently hypomethylated MTase motif sites, across isolates, only loci present in at least half of the clinical isolates were included;

FIG. 17A graphically illustrates distribution of standard deviation size of kinetics (log 2 of the IPD Ratio) for each common (n>50) motif sites across isolates with the relevant MTase active; and

FIG. 17C graphically illustrates point plot of each common motif site position according to its mean and standard deviation across isolates with active MTases. Points are colors according to whether they are hypomethylated (blue), hypervariable (red), hypervariable and hypomethylated (purple), or meet none of these criteria (grey). The top 5 most variable motif sites and bottom 5 mean sites for each MTase are labelled, if they classified as hypervariable and/or hypomethylated;

as described in further detail in Example 1, below.

FIG. 18 graphically illustrates histogram showing sigma Factor Binding Site Motif and MTase Motif overlap; where overlap of MTase and SFBS motifs for M. tuberculosis Sigma factors, and the histogram height corresponds to the number of TSSs harboring an overlap at that position, as described in further detail in Example 1, below.

FIG. 19 illustrates a table showing the activity of observed methyltransferase genotypes; for each distinct methyltransferase (MTase) variant found in our M. tuberculosis isolates, the resulting sequencing kinetics signals of bases targeted by the MTase motif in that isolate were measured, and from them inferred the activity of the variant MTase, as described in further detail in Example 1, below.

FIG. 20 illustrates a table showing that out of the 4,486 shared MTase motif sites, 351 had variation at least three standard deviations above the mean variation among MamB sites, as described in further detail in Example 1, below.

FIG. 21 illustrates a table showing anomalous methylation patterns in orphan MTase motif sites, as described in further detail in Example 1, below.

FIG. 22 Transcription Factor Binding Motifs (TFBSs) overlapping with methylation motif sites, as described in further detail in Example 1, below.

FIG. 23 illustrates a table showing loci targeted by base-modifying enzymes of M. tuberculosis that are positioned to alter expression of genes responsible for differential resistance within the M. tuberculosis bacteria, as described in further detail in Example 1, below.

FIG. 24 illustrates a table showing genes that were differentially expressed between hsdM_{WT} and ΔhsdM (ΔhsdM-DE), and analysis of RNAseq data in wild-type versus HsdM-knockout demonstrates direct transcriptional influence by HsdM promoter methylation, as described in further detail in Example 1, below.

FIG. 25A-B illustrate that intercellular mosaic methylation (IMM) is distinct from other forms of mosaic-like DNA methylation, including a conceptual illustration contrasting DNA methylome diversification and epigenetic inheritance between IMM and other mosaic-like mechanisms of heterogeneous DNA adenine methylation:’

FIG. 25A schematically illustrates the nature of methylomic diversity depicts individual cells' chromosomes (gray bars) with methylation motifs (ovals), and oval colors represent distinct DNA methyltransferases (MTases); and

FIG. 25B schematically illustrates the relationship between daughter and parent strains as it relates to conservation of the whole methylome (top) and at a single methylation site (bottom);

as described in further detail in Example 2, below.

FIG. 26 illustrates an exemplary methylation-dependent restriction fragment length scheme for an epigenomic diagnostic device, as described in further detail in Example 2, below.

FIG. 27A illustrates the association between estimated methylated fraction (scaled IPD ratio) and resistance phenotypes; and

FIG. 27B illustrates INH resistance conferred by different genotypic mechanisms clustered by methylation level at two motif sites,

as described in further detail in Example 2, below.

The patent or application file contains at least one drawing executed in color. Copies of this patent or patent application publication with color drawing(s) will be provided by the Office upon request and payment of the necessary fee.

Like reference symbols in the various drawings indicate like elements.

### DETAILED DESCRIPTION

In alternative embodiments, provided are compositions, including products of manufacture and kits, and methods, for treating or ameliorating a Mycobacterium tuberculosis (TB) or a Mycobacterium africanum infection.

We have identified 371 M. tuberculosis genomic loci in gene promoters that are modified heterogeneously within clinical isolates. This affects gene expression, through their interaction with effectors of transcription. Of the identified loci, 63 fall within promoters of 33 genes whose products influence clinically important phenotypes (drug persistence, resistance, and tolerance). Heterogeneous modification at these loci can cause differential expression of these genes across bacilli of a population. Those bacilli whose differential expression offers an advantage under the environmental pressures (e.g. drug pressure) will survive and propagate, reducing drug treatment efficacy in infections that appear genetically susceptible to the prescribed antibiotics.

The fully de novo assembled set of DNA methylomes and genomes we have generated comprise the largest such set for M. tuberculosis, and, to our knowledge, any pathogen. This analysis was enabled by our unique methods of assembly and annotation, enabling comprehensive identification of these the modification sites for the first time. We have verified that these sites are present across global strains of M. tuberculosis and identified a subset of the sites described above that are positioned to modulate transcription of genes that are present across all or most clinical isolates examined, a challenging key step toward demonstrating their utility as therapeutic and diagnostic targets. Moreover, we have created methods for measuring their propensity to vary across members of the bacterial population isolated from a patient, demonstrating utility in prognostics that assess the capacity of a sample to persist until more permanent mechanisms of resistance to therapeutics emerges. We have identified a specific set of 33 genes whose expression influences key clinical phenotypes and are affected by methylation. These genes include efflux pumps and their regulators that influence drug tolerance, regulators of metabolic downshifts shown to induce a dormant phenotype, toxin-antitoxin modules that induce persistence through post-transcriptional mechanisms, and genes encoding products known to dictate resistance levels through both intrinsic and acquired mechanisms, and their regulation.

Described herein are molecular targets in the M. tuberculosis genome for the development of drugs, diagnostic tools, prognostic indicators, and clinical decision support for TB infection. These sites have been invisible to scientists despite widespread DNA sequencing because they operate above the DNA level, through the addition of chemical tags to DNA. These tags can change how much of certain genes in the bacteria are used. The use of these affected genes can help the bacteria withstand drug treatments without dying and are undetectable with existing diagnostics. Prioritizing these sites as targets for developing drugs, diagnostics, and prognostics hold promise to improve the toolkit available to doctors to effectively treat TB patients, and epidemiologists to better control the TB pandemic, which kills more adults than Malaria, AIDS, and all tropical diseases combined. This improved toolkit will enable doctors to make more informed treatment decisions and infectious disease scientists to more effectively control TB outbreaks.

Provided herein is a method of epigenetic diagnostics that can measure the markers that change rapidly (within a day) in response to the environmental and drug pressures. As such, our approach can be used as soon as the first day of treatment for detecting drug resistance and persistence that genetic markers are able to detect months later.

Most if not all bacteria, including pathogens, possess nucleic acid modifying enzymes. Nucleic acid (base) modification refers to the addition of chemical species to a DNA base. When “epigenetic mosaicism” is occurring, these enzymes modify their target nucleic acids incompletely within each cell, giving rise to a subpopulation of bacteria giving rise to a mosaic of modified and unmodified DNA bases in bacteria within infected tissues. The modification status at these bases can alter the phenotypes of infecting bacteria in clinically meaningful ways, affecting treatment outcome. “Epigenetic mosaicism” is our discovery and we have coined it as such in Example 1 for the first time.

We use DNA methylation as the example base modification throughout this document; the DNA base modification is used to demonstrate the principle of this invention. However, methods as described herein are not exclusive to DNA methylation, but can applies to other DNA base modifications as well. When we use the term “intercellular mosaic methylation”, the principle can be similarly applied to mosaic base modifications of other chemical species, the portion of the method involving inferring intercellular mosaic methylation from genotype, however, is restricted to base modifications conferred by genetically encoded mechanisms. We use sequencing kinetics as the signature for measuring modification from Pacific Biosciences SMRT-sequencing, and principles for using the measured current changes are from Oxford Nanopore data.

Intercellular mosaic methylation is a previously undescribed form of epigenetic heterogeneity where the base modification is DNA methylation. Intercellular mosaic methylation emerges from the previously described “intracellular stochastic methylation”, with the additional knowledge that kinetics across reads mapping to a particular site displays average kinetics that resemble neither invariable methylation, nor invariable non-methylation. This knowledge, in combination with observed intracellular stochastic methylation, imply a diverse array of combinations of methylated and nonmethylated sites across the cells the DNA sequences originated from. This diversity of combinations of methylated bases is what we refer to as “intercellular mosaic methylation.” Intercellular mosaic methylation is notably distinct from the comparatively well-described phenomenon of phase variant methylation (FIG. 4G), in which a subpopulation of cells have an inactive DNA methyltransferase, and the other subpopulation has an active DNA methyltransferase. In phase variant methylation, some cells have all methylation sites unmethylated, while others have (nearly) all methylated. The key difference of clinical importance between phase variant methylation and intercellular mosaic methylation is that phase variant methylation creates two distinct phenotypes that can be selected for, while intercellular mosaic methylation creates a spectrum of phenotypic diversity, providing far more opportunities to create a phenotype that can persist or resist environmental and drug pressures.

For example, see FIG. 4G, schematically illustrating how intercellular mosaic arises from stochastic methylation, this figure is a conceptual illustration depicting the distinction between methylome diversity within colonies exhibiting phase-variable methylation (top) and stochastic Methylation (bottom). Each gray segment represents chromosome from an individual cell within the colony. Each oval within the segment represents a methylation locus, illustrated as methylated (mint) or unmethylated (red).

These different methylation combinations cause differential phenotypes between members of infecting bacterial population. Based on this realization, provided herein are methods to:
1. Identify “intercellular mosaic methylation” from sequencing kinetics of isolated DNA from an organism, and the genetic or environmental factors that result in intercellular mosaic methylation. Subsequent steps are explained using the sequencing kinetics data, but the principle holds for Nanopore data as well, using current instead of kinetics.
   a. Identify the presence or absence of intercellular mosaic methylation from sequencing kinetics;
   b. Incorporating genomic data with (la) to identify Gene alleles for base-modifying enzymes that confer constitutive epigenetic mosaicism;
   c. Environmental or genetic manipulation prior to sequencing to identify mutations, nutrient constraints, and stressors that cause or induce intercellular mosaic methylation.
2. Identify DNA bases (loci) that will be differentially affected (because they are methylated in some cells and not in others) across cells of the sequenced population.
3. Incorporate genomic annotation data to identify loci from (2) that are phenotypically consequential. For pathogens, the focus is on loci identify affecting clinically important phenotypes (e.g. drug tolerance, resistance, persistence, and entry to dormancy).

We have applied methods as provided herein to Mycobacterium tuberculosis (M. tuberculosis), the primary bacterial cause of Tuberculosis, which killed more humans (1.5 million) than any other infectious disease in 2018. Applying our methods to M. tuberculosis revealed several dozen loci targeted by base-modifying enzymes of M. tuberculosis and are positioned to alter expression of genes responsible for differential resistance within M. tuberculosis bacteria (in Example 1, see Table 1, FIG. 23). This method can be used to obtain both the specific sites in the genome that may modulate antibiotic resistance levels among infectious bacteria and identify the propensity of a particular strain to possess intercellular mosaic methylation within a patient. This information can be used to infer heteroresistance within clinical samples, and subsequently inform treatment regimens by physicians, and to inform containment practices in cases of disease outbreaks caused by bacterial pathogens. As such, the methods behind this invention can be used to develop diagnostic, prognostic, and Clinical Decision Support (CDS) tools.

Example 1 describes intercellular mosaic methylation affecting areas of the genome in a bacterial pathogen, Mycobacterium tuberculosis, that are positioned such that they likely alter the level at which genes are expressed (Table 2, see FIG. 9, contains a subset of such genes). This is clinically consequential when, for instance, the genes involved reduce susceptibility to antimicrobial drugs. Prior work in detecting heterogeneous methylation in bacteria has been limited to identifying the presence or absence of heterogeneity within and across reads, but had not been extrapolated to the combinations of modified sites across members of a clonal population. Nor have methods to combine this data with genomic annotation to determine clinically important modified loci been described. Our method combines heterogeneity detection levels within individual reads with information about the aggregate average of kinetics signal across reads from a bacterial population mapping to a particular genetic locus.

One of the key technical advancements described herein is using the dual presence of within-read methylation heterogeneity and the kinetic average at a single site to demonstrate mosaicism in patterns throughout the colony. We further innovated by developing methods to maps that the genotype of the modifying enzymes to basal epigenetic mosaicism and demonstrating that such mosaicking is inducible by nutritional stress in bacterial strains with base modifying enzymes that do not cause epigenetic mosaicism at baseline. Methods as provided herein also incorporate genome annotations to infer loci where modification status is most likely phenotypically consequential.

We show that intercellular mosaic methylation is 1) detectable through analysis of sequencing kinetics data, 2) that it is likely to affect expression of genes mediating survival probability under drug treatment in the bacterial pathogens responsible for the most deaths of any infectious disease agent in the world, Mycobacterium tuberculosis 3) that it can be caused by genotype of base-modifying enzymes or induced by nutrient starvation.

We have applied a computational pipeline to Mycobacterium tuberculosis. Through this, we discovered intercellular mosaic methylation (a form of epigenetic heterogeneity), and that it is constitutively present in some strains of M. tuberculosis isolated from patients, and absent in others. Moreover, we have discovered that this constitutive intercellular mosaic methylation is determined by genotype (See FIGS. 4 and 15) of DNA methyltransferase enzymes and characterized the profiles for 42 alleles of three known DNA methyltransferases (MTases) in the M. tuberculosis genomes. The results described in Example 1 demonstrate the presence of constitutive intercellular mosaic methylation and determines the breadth of the spectrum of clinically relevant phenotypic diversity in TB infection in humans. Therefore, this cataloging of MTase allele relationship to constitutive intercellular mosaic methylation is valuable for informing treatment plans and TB control strategies during outbreaks.

We describe several hundred loci that are targeted by MTases and positioned to alter expression of genes through their effect on promoter strength and interaction with various molecular effectors of M. tuberculosis transcription. These include influencers of persistence, drug resistance, and drug tolerance M. tuberculosis. This catalog of MTase allele relationship to constitutive intercellular mosaic methylation allows development of diagnostic and prognostic tools of heteroresistance and persistence in M. tuberculosis through targeted genotypic assays. The results of these assays will inform infection control agencies and physicians of the capacity for isolates to heterogeneously modulate antibiotic resistance, drug tolerance levels, and persister cell formation propensity on a patient-specific basis. These phenomena often occur far below the sensitivity thresholds of extant diagnostic tools, challenging informed treatment and containment protocols by current methods. Finally, we envision a new line of TB treatment using phage therapy. This radically new approach uses the results of our MTase genotype analysis to design phages that can alter the infecting cells into a state that prevents the cells from diversification (through methylation phasing or intercellular mosaic methylation).

Data described herein demonstrates that MTase genotype is consistently predictive of methylation activity level, and of constitutive intercellular mosaic methylation. Moreover, the set of M. tuberculosis clinical isolates we have studied demonstrate:
1. Differential constitutive DNA methylation affects more of the genome than single-nucleotide polymorphisms, the most commonly compared information between strains for molecular diagnostics.
2. DNA Methylation is invariably present across all five major lineages of M. tuberculosis, and highly variable between strains in a manner that is not consistent with phylogeny alone.
3. Constitutive intercellular mosaic methylation is more frequent in hypervirulent and extensively drug-resistant strains.
4. By compiling the largest library of MTase allele MTase activity mappings in the world, and through methodological advancements, we have corrected previously published mappings; see e.g., FIG. 2; see FIG. 13; and in the Table as illustrated as FIG. 19.

FIG. 19: Activity of observed methyltransferase genotypes. For each distinct methyltransferase (MTase) variant found in our M. tuberculosis isolates, we measured the resulting sequencing kinetics signals of bases targeted by the MTase motif in that isolate, and from them inferred the activity of the variant MTase, reported here. Variants that were not present in our dataset could potentially be with respect to H37Rv instead of a wildtype MTase. Orange variants in column “Chiner-Oms et al.” were labeled “Partially methylated” by Chiner-Oms et al. *R47W and G154D were only found in H37Rv and H37Ra. **Inferred to be deleterious, since only found in conjunction with D59G and V616A, which result in wild-type methylation patterns in the absence of this insertion. ***Also inferred to be deleterious, since only found in conjunction with V616A. ****K458N only found in tandem with E481A. †For hsdM variant E481A, Our study sequenced the same isolate as Chiner-oms & colleagues, but our genotyping showed both E481A and K458N in hsdM, while they only reported E481A. Both studies showed a mild knockdown effect for this isolate, but it unclear which mutation causes it, or whether the effect is epistatic.

In the study described in Example 1, the methylomes of a global collection of 93 clinical isolates from all seven lineages of the M. tuberculosis complex (MTBC) were analyzed. The sequence of each isolate was de novo assembled into complete, circularized genomes and integrated with gene, promoter, and transcription factor binding site data, see FIG. 1A-D. This is the largest intra-species comparative methylome study to date, and the first to examine all seven MTBC lineages. Our analysis revealed the following. All but one of the 35 East-Asian isolates displayed heterogeneous methylation, and shared a distinct MTase genotype. Type strain H37Rv had the rarest MTase activity profile among studied isolates, while several MTase activity profiles converged across lineages. A subset of MTase motif sites were consistently hypomethylated across isolates, regardless of MTase activity, and showed clear evidence of transcription factor occlusion. Finally, MTase motif sites were frequently within strictly defined gene promoters, including several genes known to regulate clinically important phenotypes. These findings demonstrate that DNA methylation drives phenotypic plasticity in M. tuberculosis and may mediate differential adaptive capacity between strains.

### DNA Methyltransferase Inhibitory Molecules

In alternative embodiments, provided are products of manufacture and kits, and methods, that comprise or comprise use of DNA methylation inhibitory molecules for treating or ameliorating a Mycobacterium tuberculosis (TB) infection. In alternative embodiments, the DNA methylation inhibitory molecules can comprise small molecules, inhibitory nucleic acids and antibodies inhibitory to DNA methyltransferases (MTases) including MamA, MamB, and HsdM. In alternative embodiments, a DNA methylation inhibitory molecule is used as described in Yadav M K, et al (2015) The Small Molecule DAM Inhibitor, Pyrimidinedione, Disrupts Streptococcus pneumoniae Biofilm Growth In Vitro. PLoS ONE 10(10): e0139238; and as illustrated in FIG. 10.

### Products of Manufacture and Kits

Provided are products of manufacture and kits for practicing methods as provided herein, including DNA methylation inhibitory molecules, including for example, small molecules, inhibitory nucleic acids and antibodies inhibitory to DNA methyltransferases (MTases) including MamA, MamB, and HsdM.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: Epigenetic Mosaicism in Human Pathogen Mycobacterium tuberculosis Permits Rapid Adaptation without Genetic Mutation

This study analyzes the methylomes of 93 Mycobacterium tuberculosis complex clinical isolates, representing seven lineages, the largest to date from a single species. By integrating DNA methylation data with fully annotated, de novo assembled finished genomes, we uncovered three key findings. First, gene promoters are frequently methylated, including the promoters of notable resistance and dormancy regulators. Second, isolates from different lineages often share methyltransferase activity profiles, demonstrating epigenetic similarity between genetically distant strains, yet few isolates match type strain H37Rv. Finally, intracellular stochastic DNA methylation generates a mosaic of methylomes within isogenic colonies, increasing phenotypic diversity. This “intercellular mosaic methylation” was driven by methyltransferase mutations in 40 isolates and could also be induced by methionine starvation. Mutation-driven intercellular mosaic methylation was most prevalent in the Beijing sublineage, potentially contributing to its global success. Intercellular mosaic methylation provides an epigenetic mechanism of phenotypic plasticity in M. tuberculosis, demonstrating an adaptive strategy previously undescribed in pathogens.

### Results

Approach: We sought to compare methylomes across a global collection of mostly M/XDR clinical isolates (FIG. 1a) to determine how DNA methylation influences M. tuberculosis phenotype. We executed this comparison through four key strategies. First, we used finished genomes and hybrid annotations to transfer annotations from the well-studied virulent M. tuberculosis type strain H37Rv and annotate genes it lacked. This retained syntenic relationships and provided facile comparative analyses. Second, we integrated transcription start sites (TSSs), transcription factor binding sites (TFBSs), and sigma factor binding sites (SFBSs) into our methylome analyses (FIG. 1B). This helped us identify promoter methylation, explain hypomethylated bases, and examine regulatory interactions with MTase motif sites. Third, we included the sequencing kinetics at all MTase motif sites in each isolate (FIG. 1B). This contrasts with the approach of prior M. tuberculosis methylome studies^{4,11,16,} which examined only the MTase motifs identified by PacBio SMRT Analysis (KineticsTools)¹⁷ in each isolate. Our inclusive approach revealed MTase genotypes with knocked down function previously mischaracterized as complete knockouts. Finally, we used phylogenetics to identify convergent MTase activity across evolution and heterogeneity analysis to assess the capacity for epigenetic change within a colony (FIG. 1c).

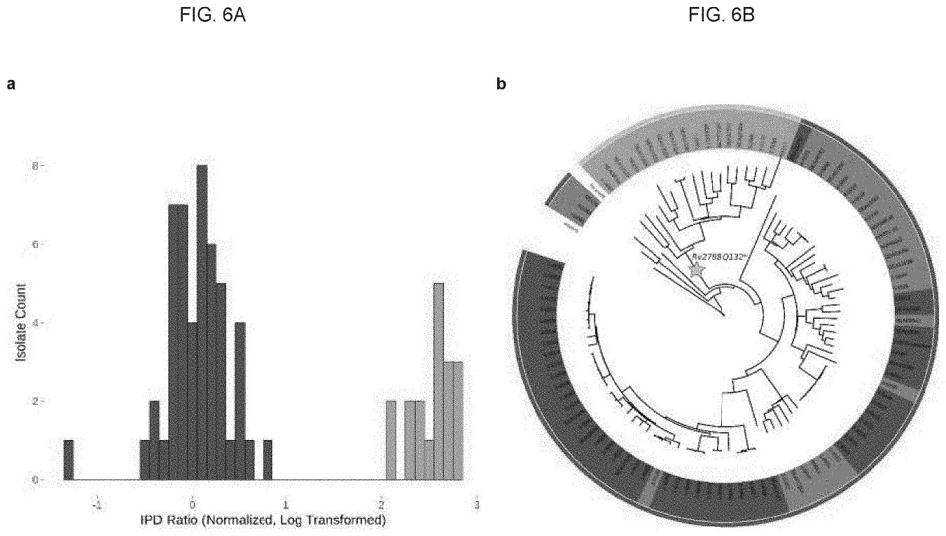

FIG. 6. Study design and approach to whole-methylome analysis. a, Isolate selection. M. tuberculosis clinical isolates were obtained from tuberculosis patient sputa from four countries of high TB-burden (India, Moldova, South Africa, and The Philippines), and Sweden (primarily isolated from migrants originating in high TB-burden countries). Isolates were cultured, and DNA extracted and sent to the Genomic Medicine Genomics Center at UCSD for amplification-free sequencing (PacBio RSII, P6C4 chemistry). Clinical isolates were supplemented by technical replicate control runs of avirulent reference strains, and publicly available clinical isolates along with technical triplicates of H37Rv (BioProject Nos. PRJNA555636, PRJNA329548, PRJEB8783). b, Methylome assembly and annotation. Raw kinetic data were transformed, scaled, and standardized according to run-specific statistics in unmodified bases (FIG. 14). Variation between technical replicates were used to adjust priors based on coverage to build pdfs for a Bayesian classifier. Methylation status of characterized motifs were classified with a Bayes' classifier based on kinetics of unmodified and modified bases for each motif (Methods). We processed all motifs of identified MTBC complex methyltransferases³ and assembled the methylome of each isolate with each motif site classified as methylated, hypomethylated, or indeterminate. We annotated each assembled methylome with overlapping and proximal features from our AnnoTUB assembly pipeline (see companion paper in this issue) annotations. We consulted annotations transferred from RATT¹⁸ to catch recurring patterns at motif sites present in virulent type strain H37Rv. c, Methylomic variation. We mapped MTase genotypes to methylation levels of their motifs to describe novel wild-type, knockdown, and knockout mutations responsible for varying degrees of motif methylation. We analyzed heterogenity with SMALR¹⁵ to characterize the capacity for methylomic variation within isogenic colonies and to probe for phase variation. We applied phylogenetic analysis of MTase genotypes and their corresponding methylation activity profiles to determine how DNA methylation across evolutionary time and identify epigenetic convergence across lineages. d, Methylome pattern discovery. We surveyed whole methylomes to identify motif sites and isolates with anomalous patterns. We examined motif sites consistently classified as hypomethylated for previously described¹⁰ causes of hypomethylation. We screened against published transcription factor binding site (TFBS) affinities¹⁹ to probe for interactions with DNA methylation. We also screened for proximal motif sites among hypomethylated bases, which can create epigenetic “switches”. Common configurations among MTase motif sites and promoter elements (transcription start sites and sigma factor binding motifs) were used to identify cis-regulatory interactions with DNA methylation.

### Epigenomic Convergence Across Lineages

For every isolate, the average Inter Pulse Duration (IPD) ratio was calculated across the reads mapping to each base¹⁷. To characterize the noise in these measurements, we compared the IPD ratios at each base between technical replicates of reference strain H37Ra (see FIG. 14).

FIG. 14: Preprocessing Quality Control. FIG. 14a, Distribution of inter pulse duration (IPD) ratios across all bases for one of the replicate runs of H37Ra. FIG. 14b, log 2 transformation converts log-normal distribution into normal distribution of log 2(IPD ratios), expressed in standard deviations from the mean (sd). FIG. 14c, Difference in sequencing kinetics between replicate H37Ra SMRT-sequencing runs across the genome. The x-axis is genome position, and the y-axis is the log 2 transformation of the IPD ratios reported by SMRT analysis for each base. Only a subsample of bases (n=100,000) are shown. Note that there were four discrepant bases the H37Ra replicates, each of which were single base insertions. This error rate (1/10⁶ or QV60) is expected of a good PacBio run at our average coverage². We removed the insertions from the discrepant run before comparing the IPD ratios between runs. The red dashed vertical line indicates the points removed. FIG. 14d, Difference in log 2(IPD ratio) between replicate runs as a function of coverage. FIG. 14e, Quantile-Quantile plot comparing IPD ratios at a subset of mamA motif (blue) and at non-mamA motifs (red) to theoretical values in a perfect normal distribution (black diagonal line). Green horizontal lines depict extremes expected to appear only once in the theoretical normal distribution.

We then identified all bases matching the known target motifs³ of established M. tuberculosis MTases and examined their IPD ratios (FIG. 2). Comparing the distribution of these IPD ratios in each isolate established their MTase activity profile, and the functional impact of MTase mutations (FIG. 2a). Each MTases had at least three distinct knockout or knockdown mutations (FIG. 2a). Phylogenetic analysis demonstrated convergent MTase activity across lineage, and diversity within lineage (FIG. 2b, 2c). The East-Asian, Euro-American, and Indo-Oceanic lineages each had multiple profiles among their members (FIG. 2c, 2d). This contradicts previous analysis on a smaller isolate set that reported lineage-specific methylation⁴.

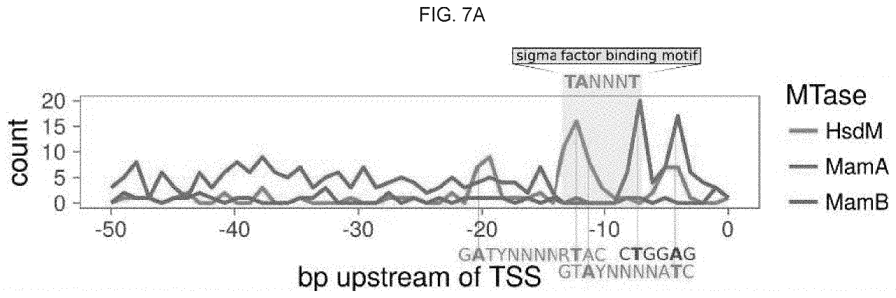

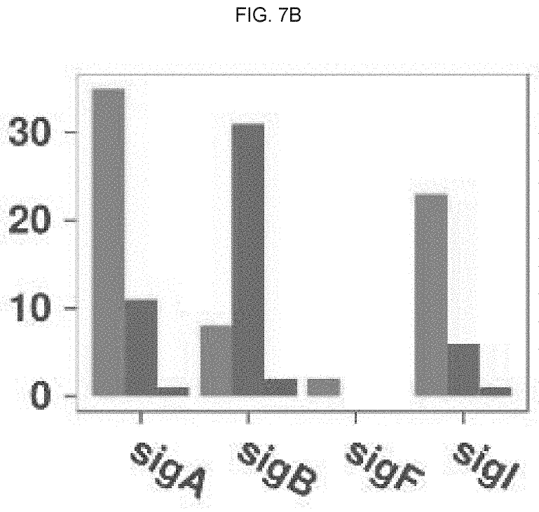

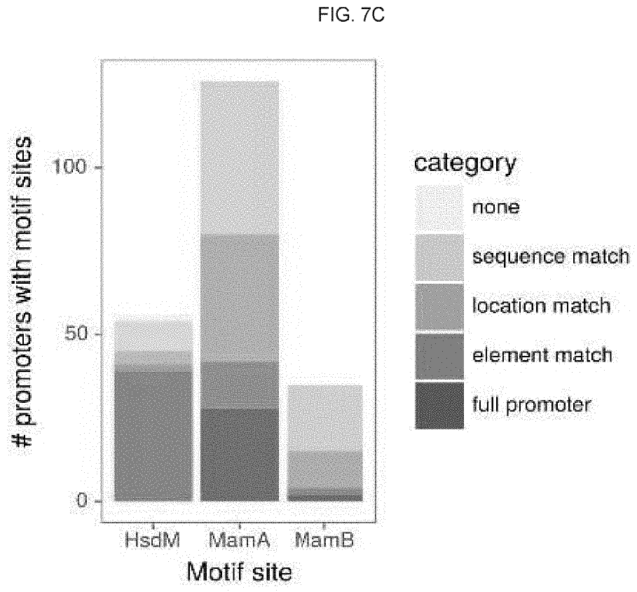

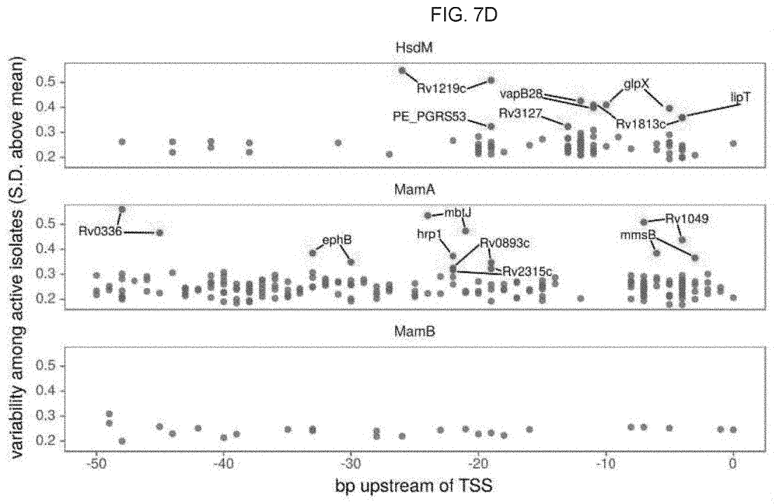

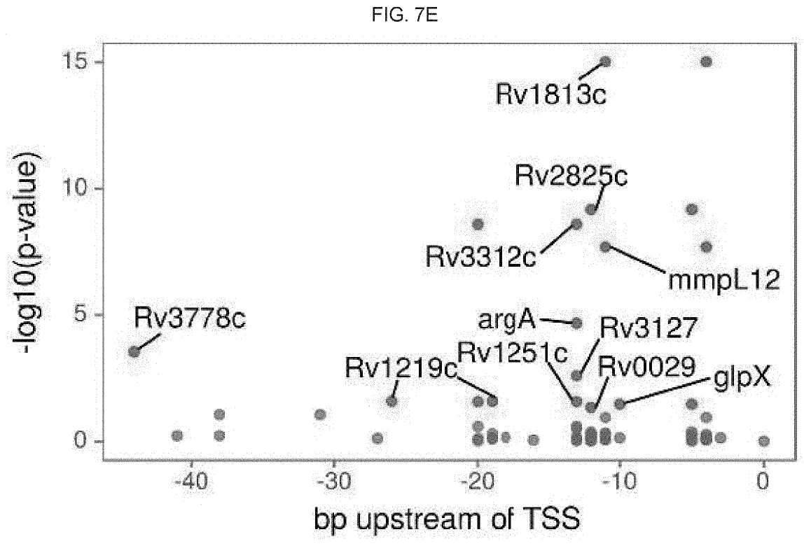

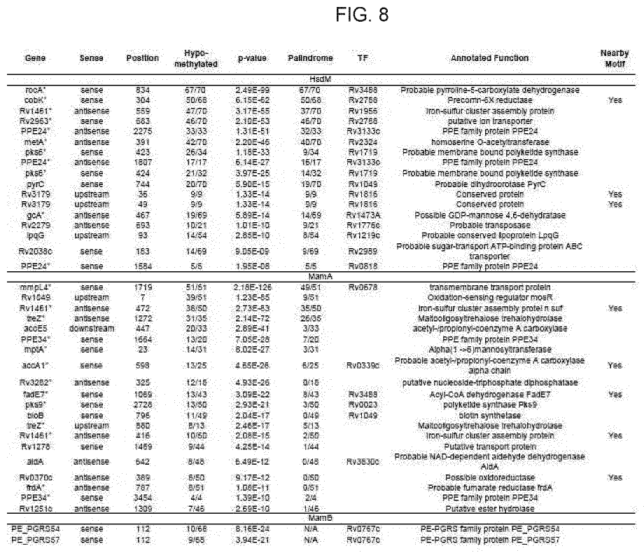

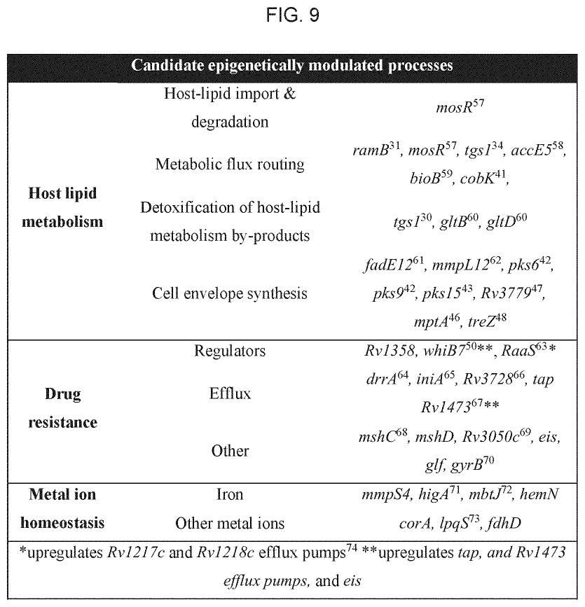

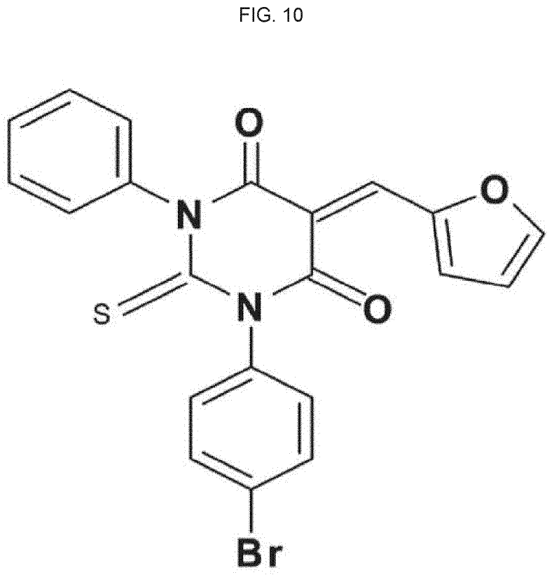

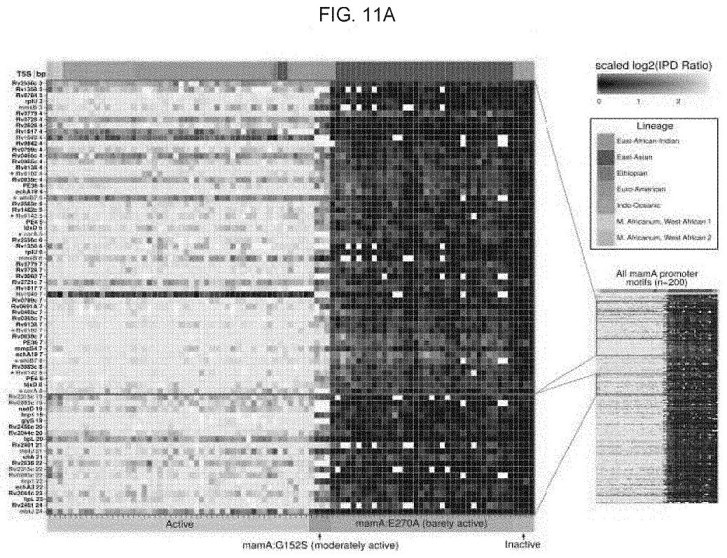

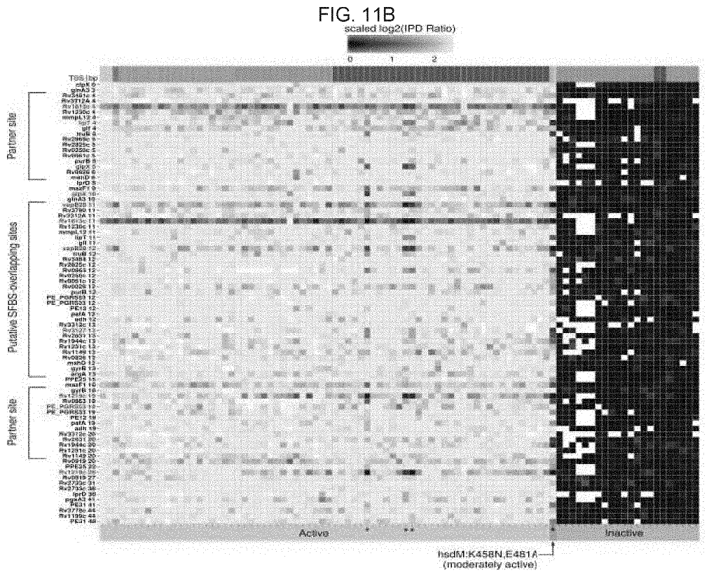

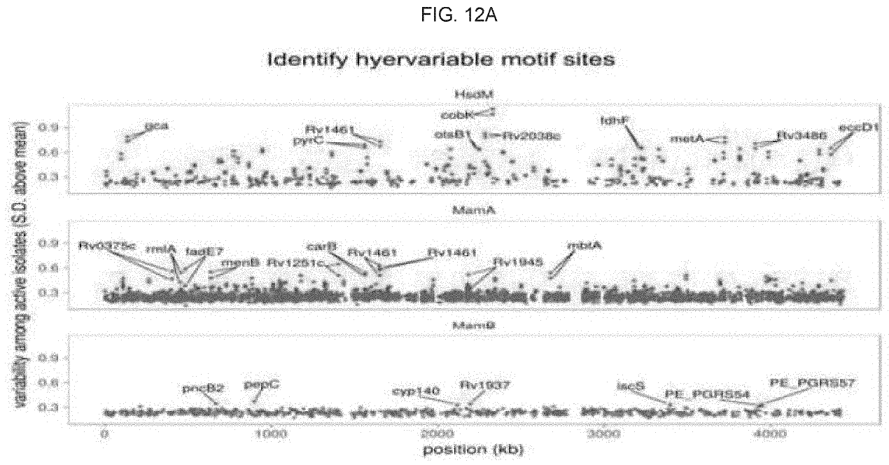

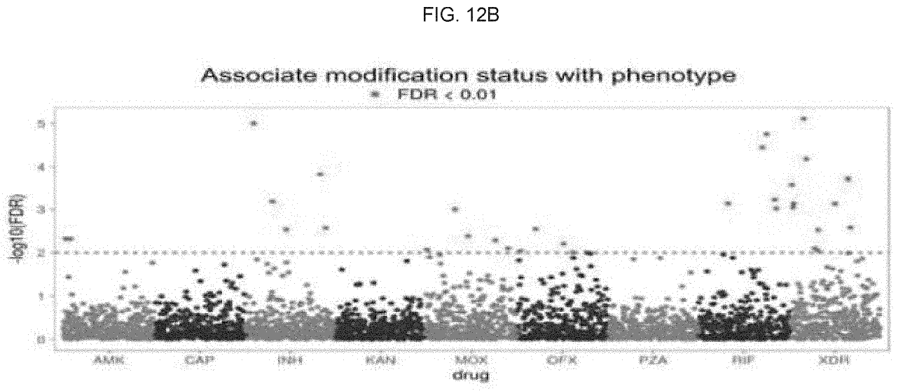

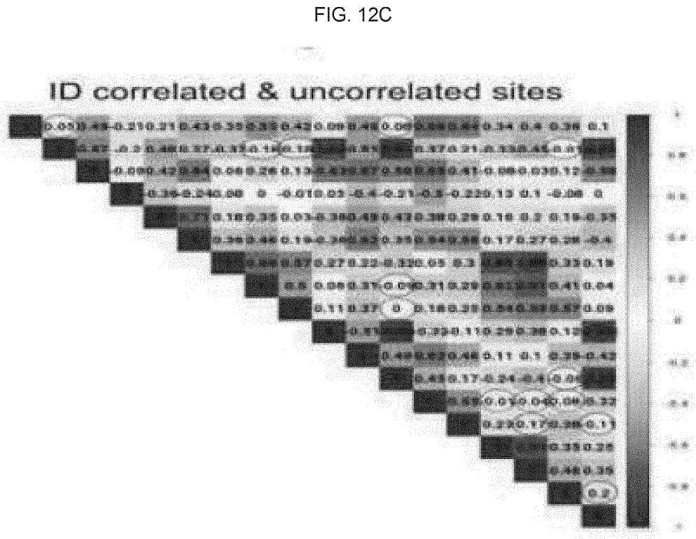

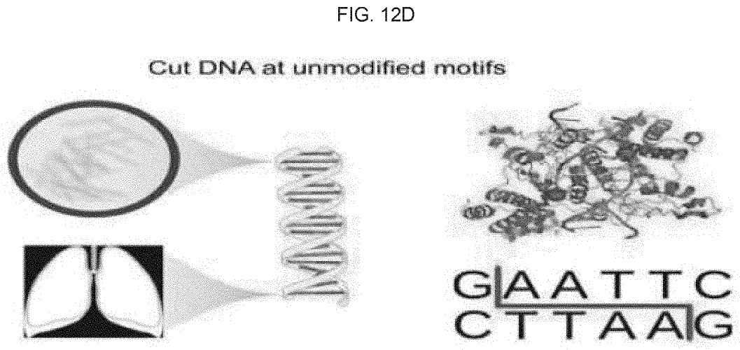

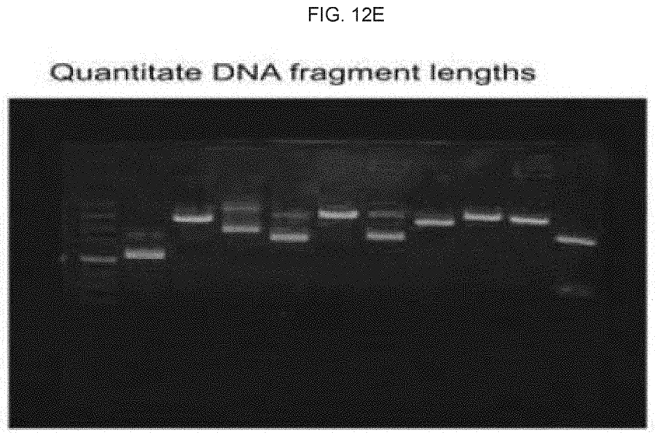

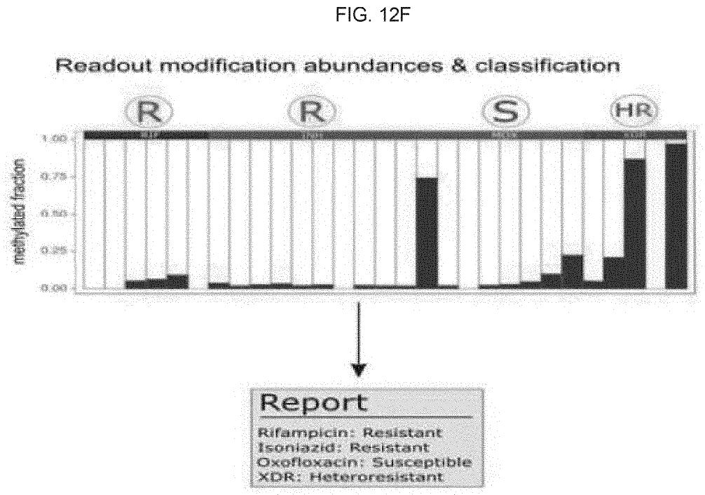

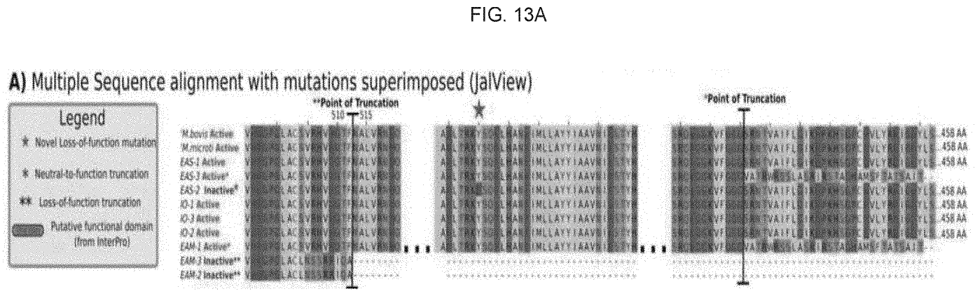

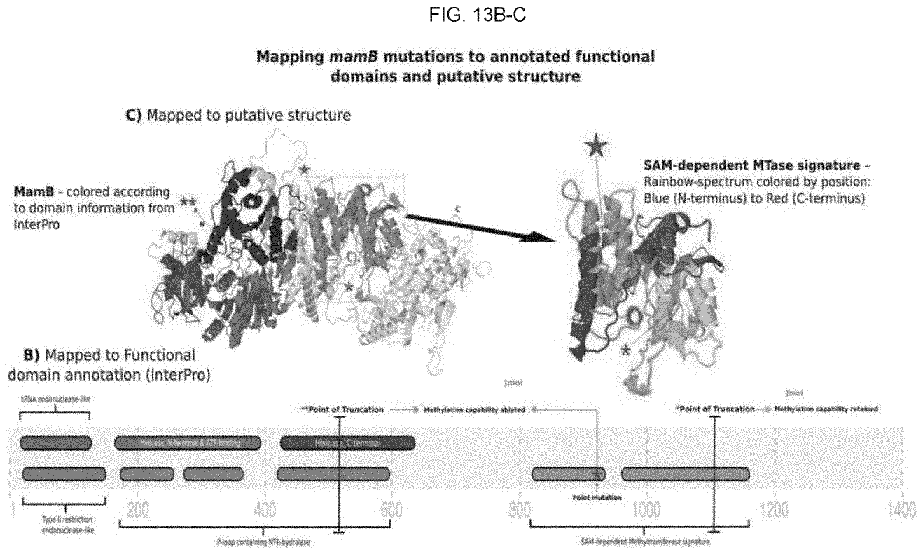

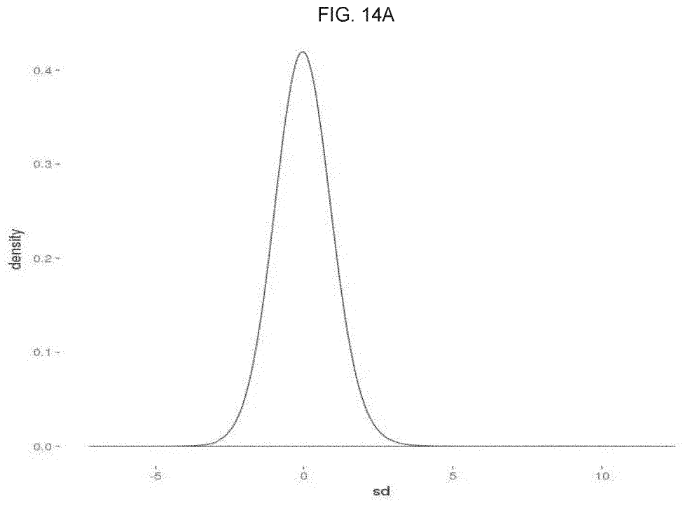

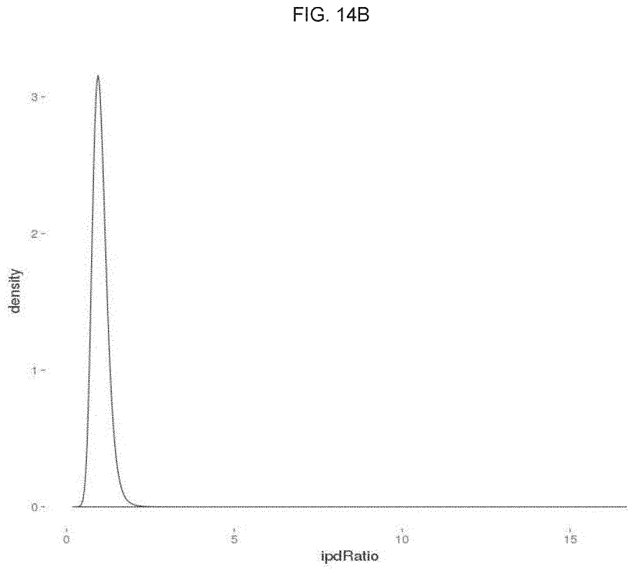

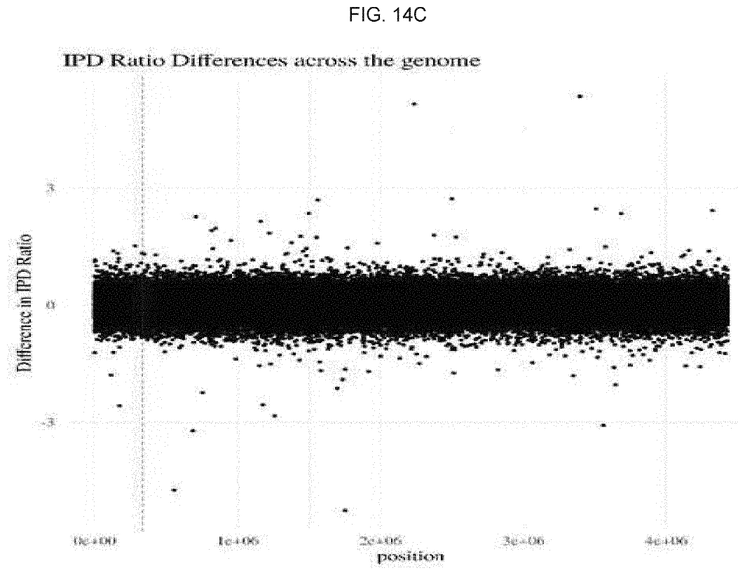

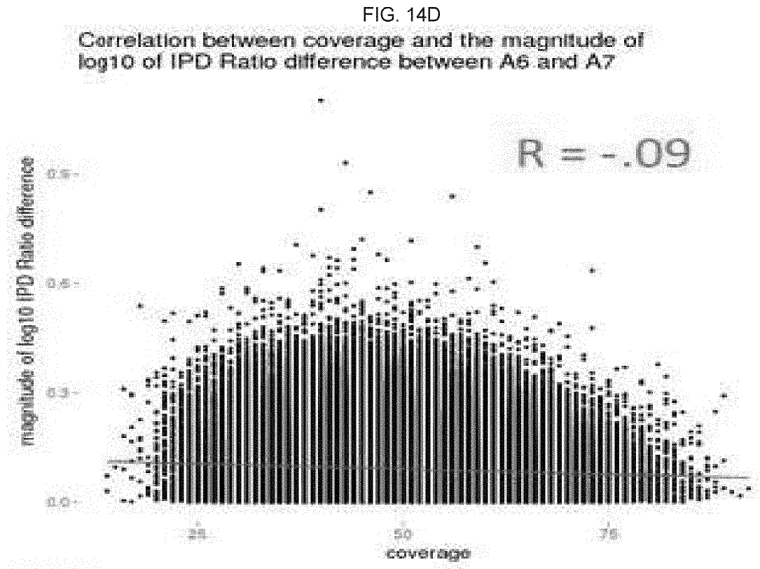

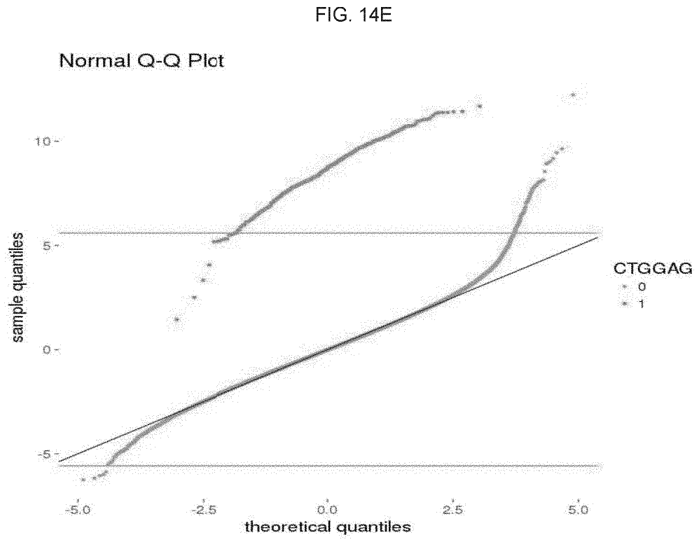

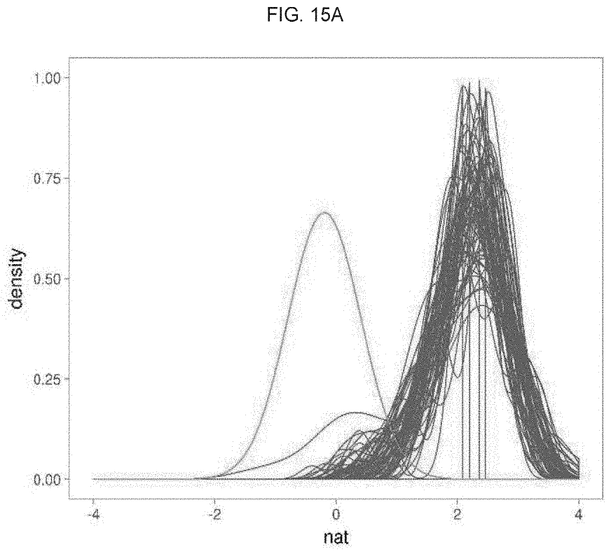

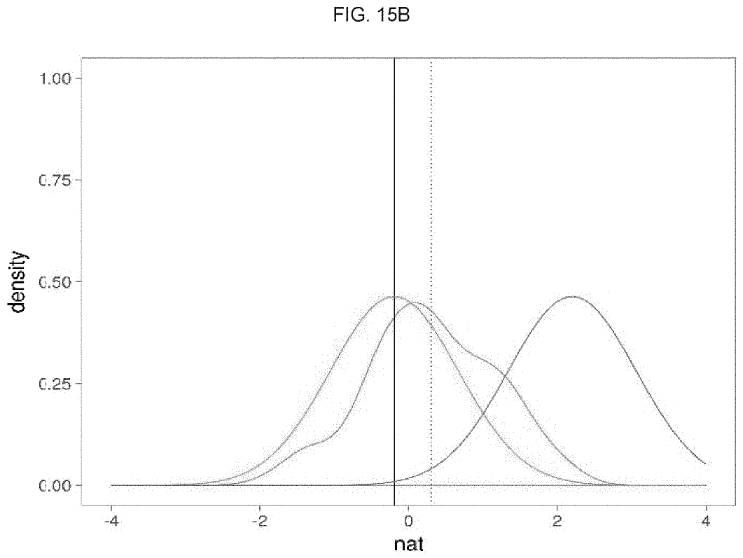

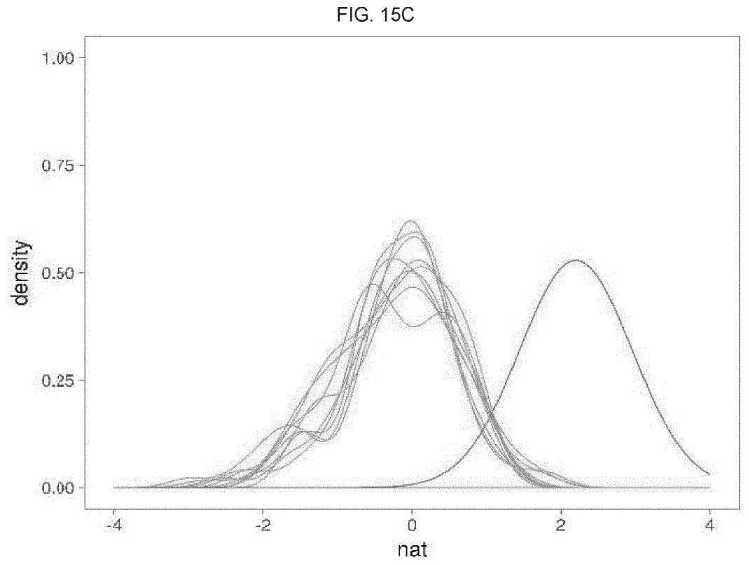

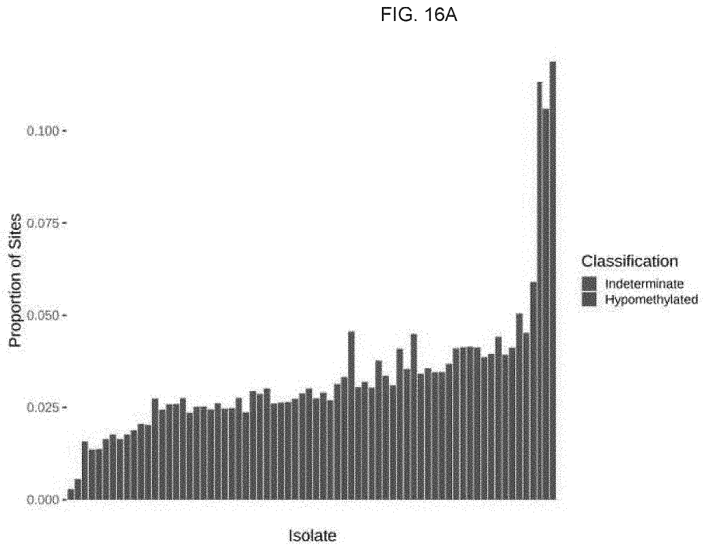

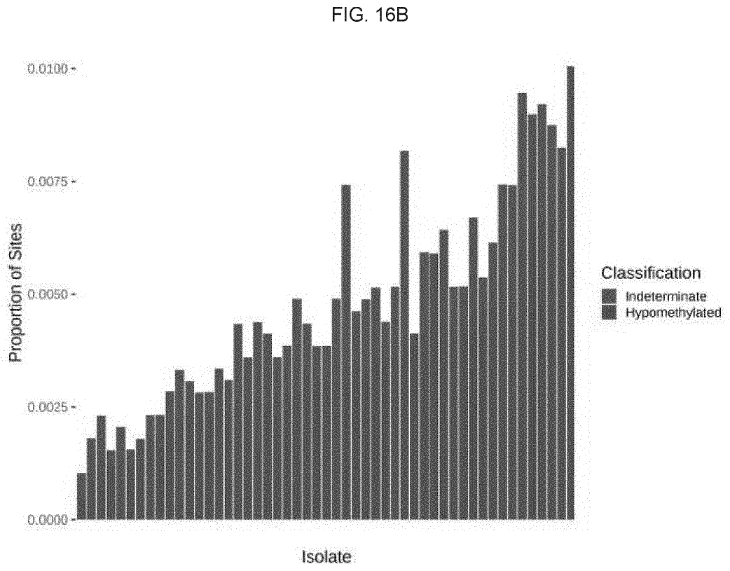

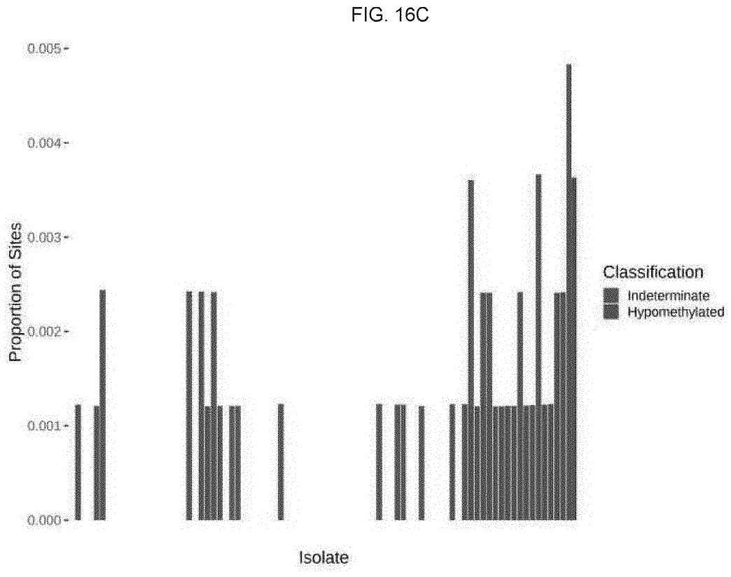

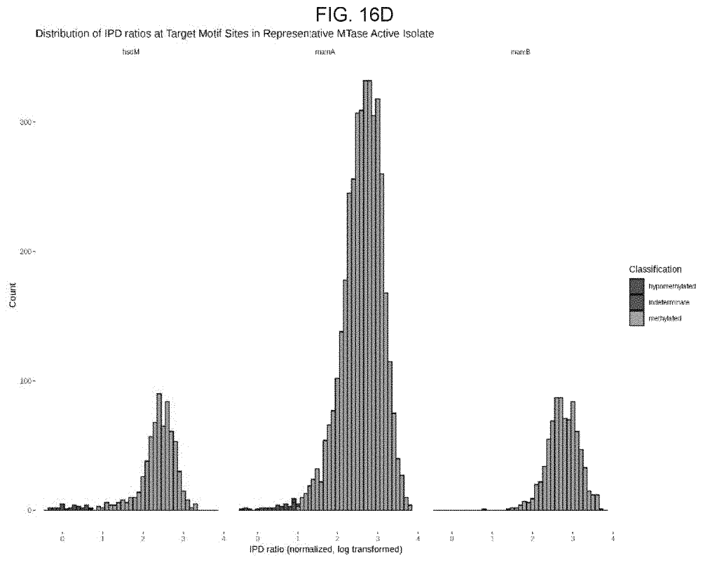

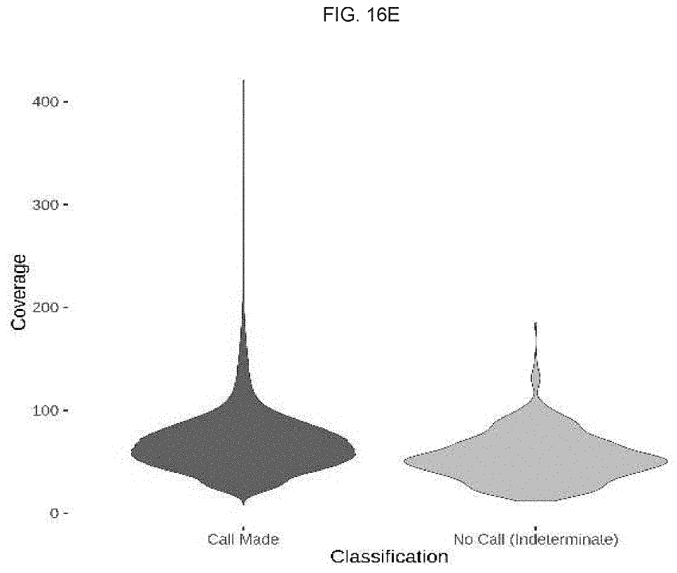

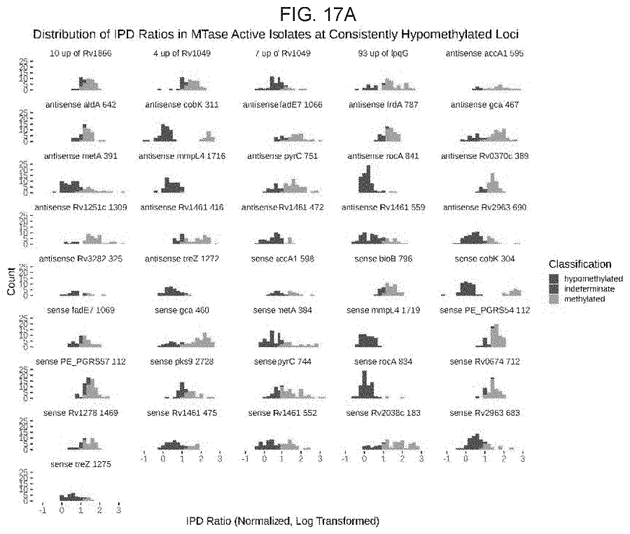

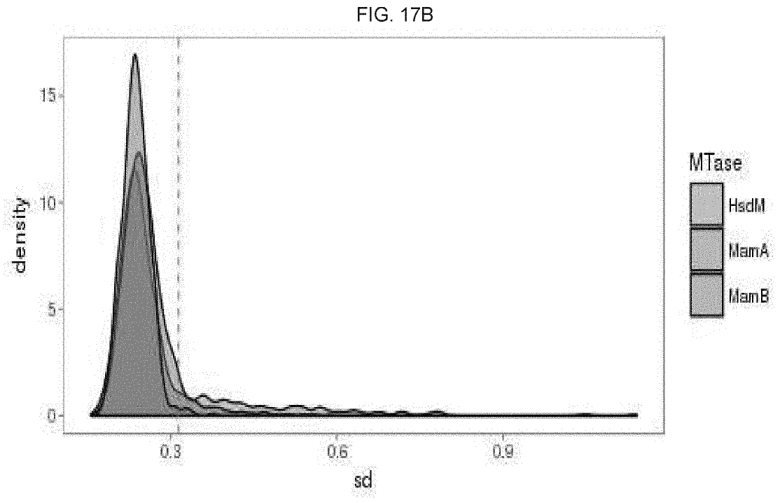

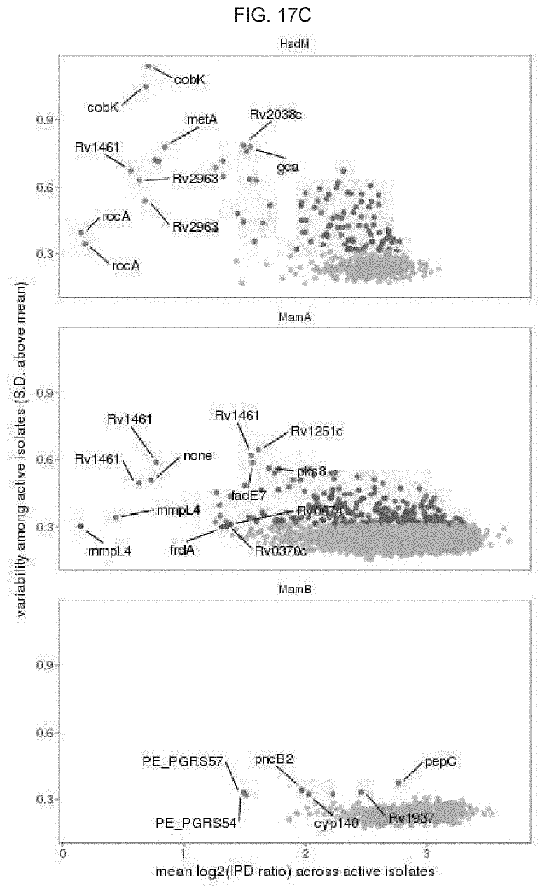

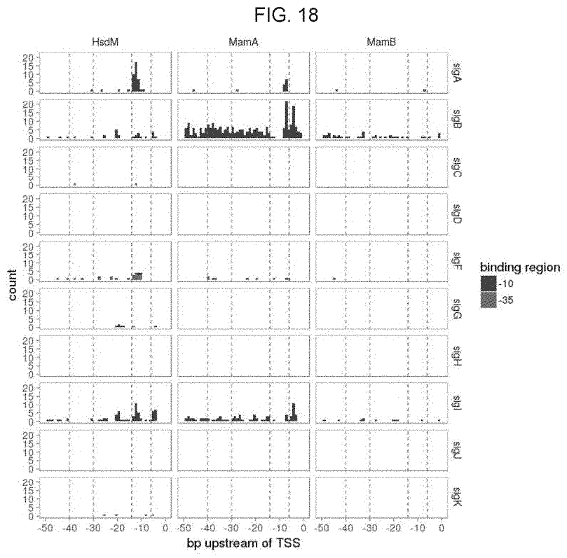

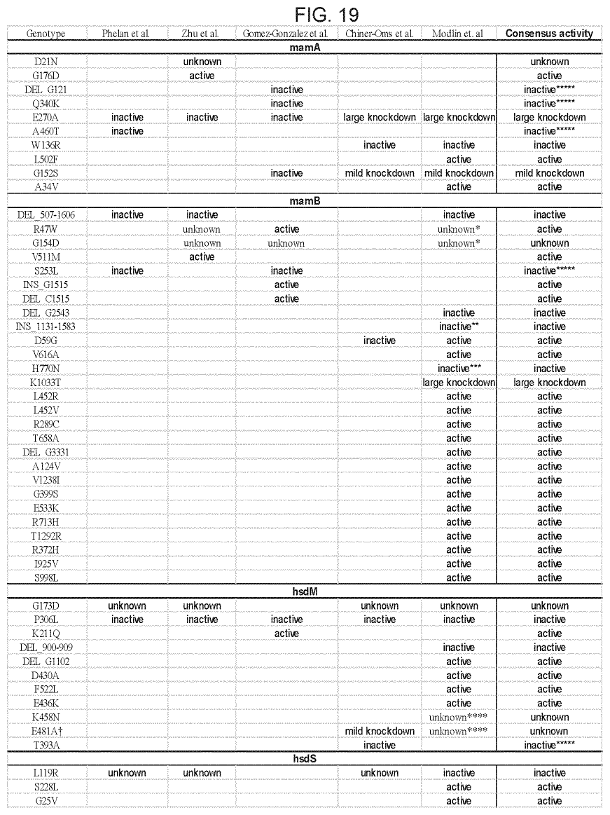

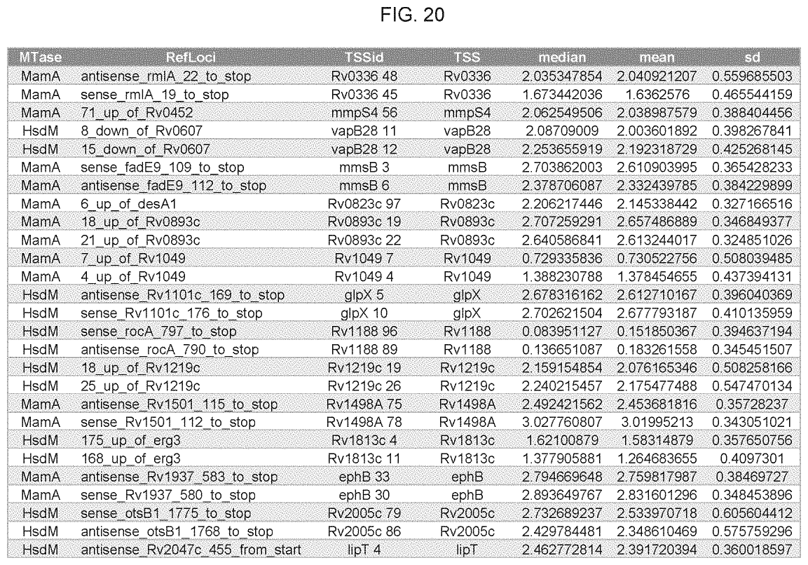

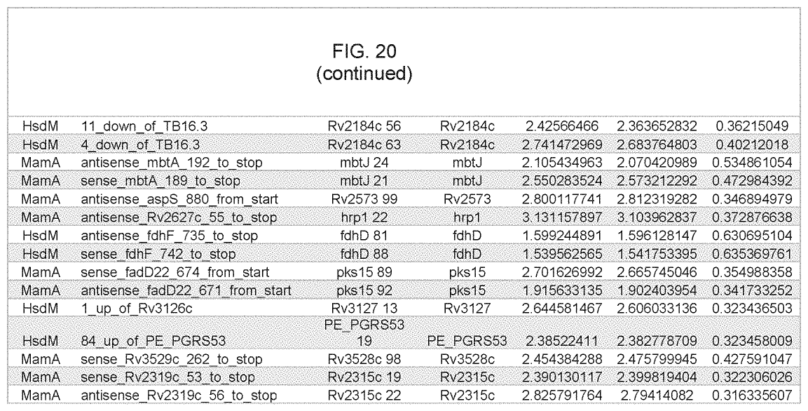

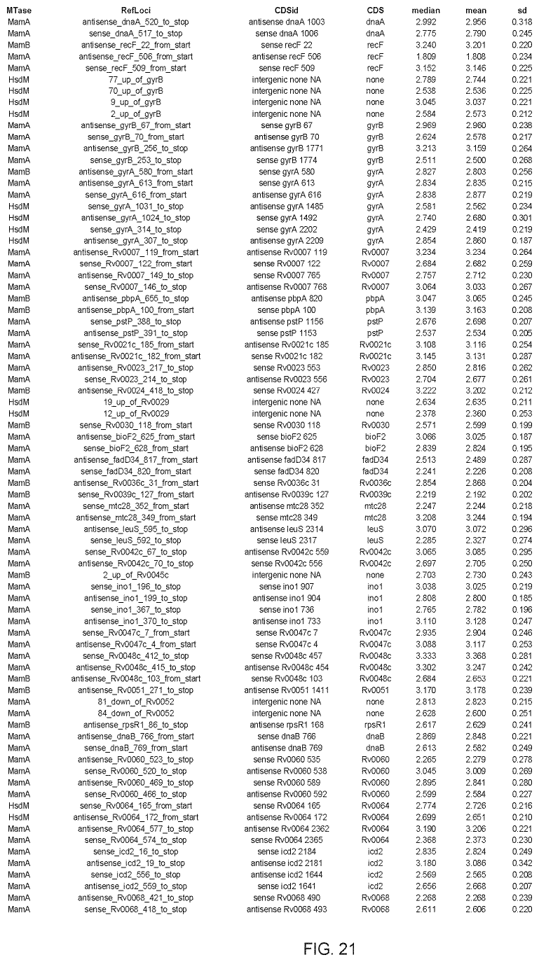

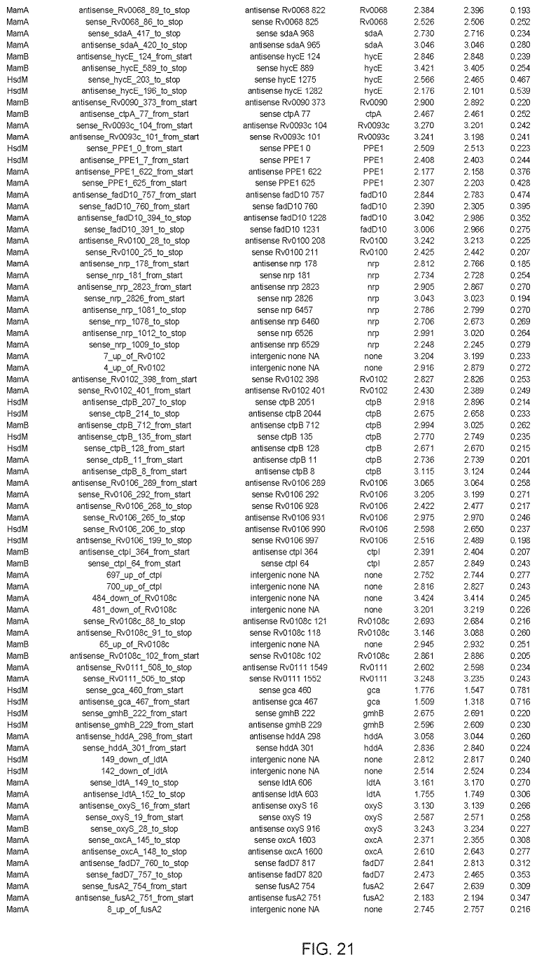

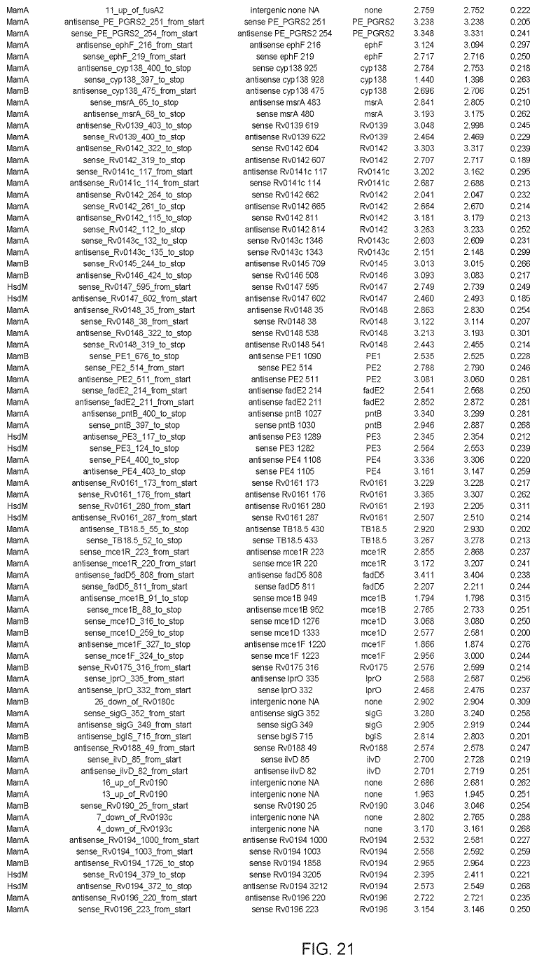

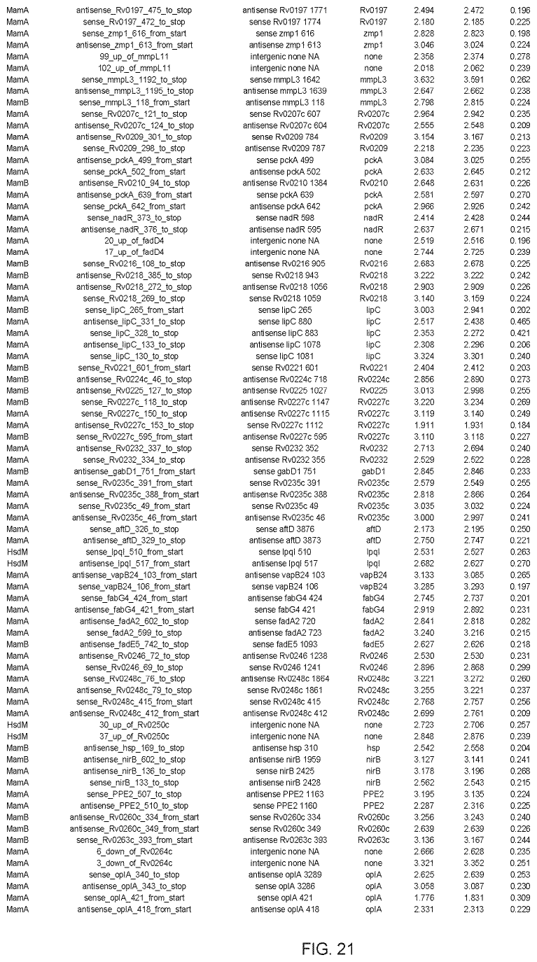

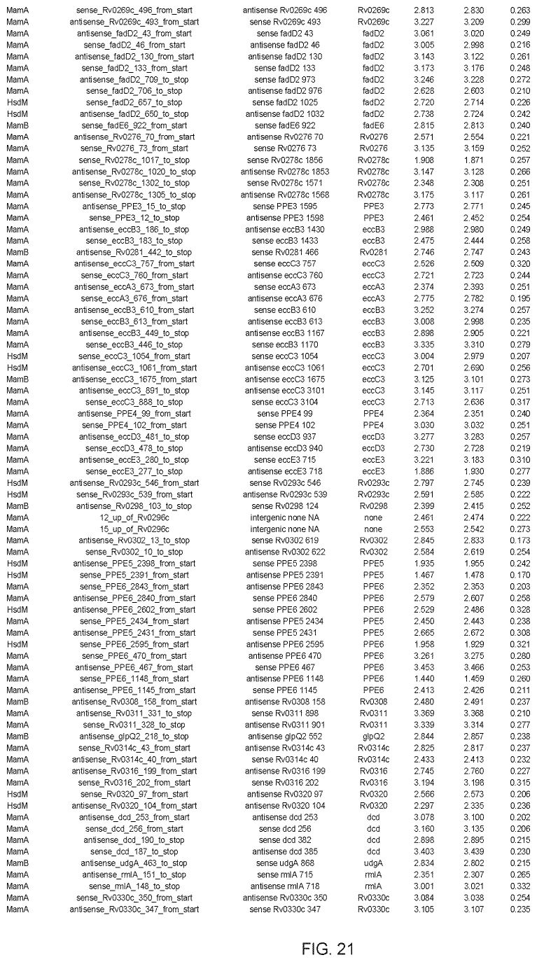

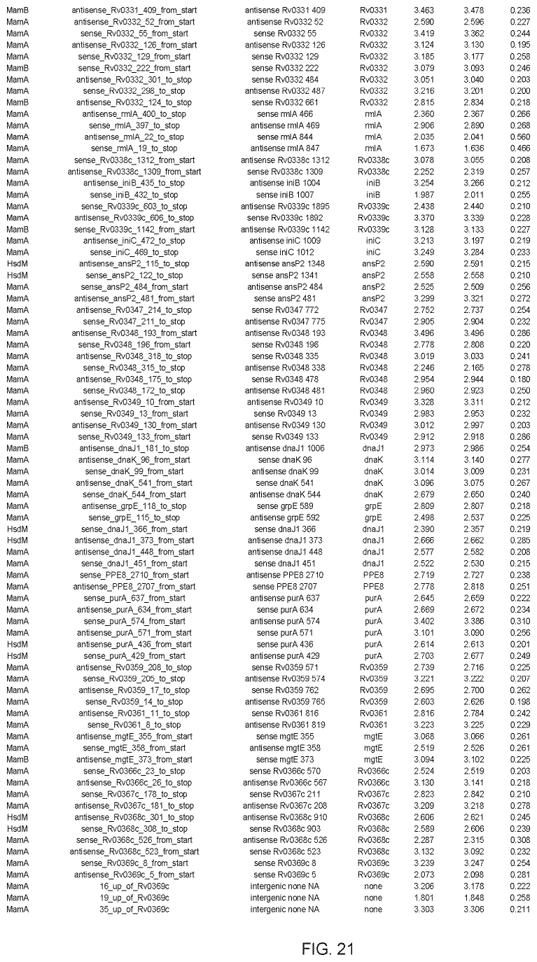

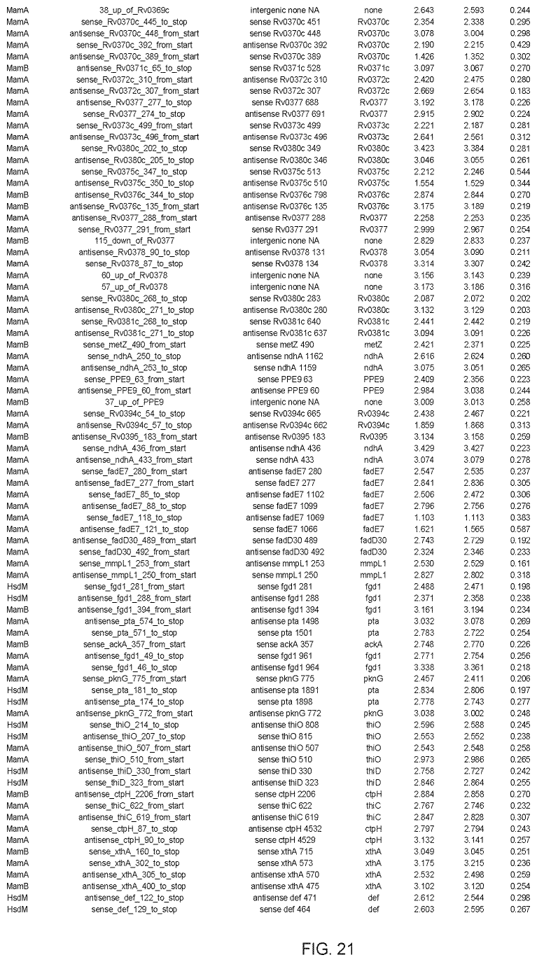

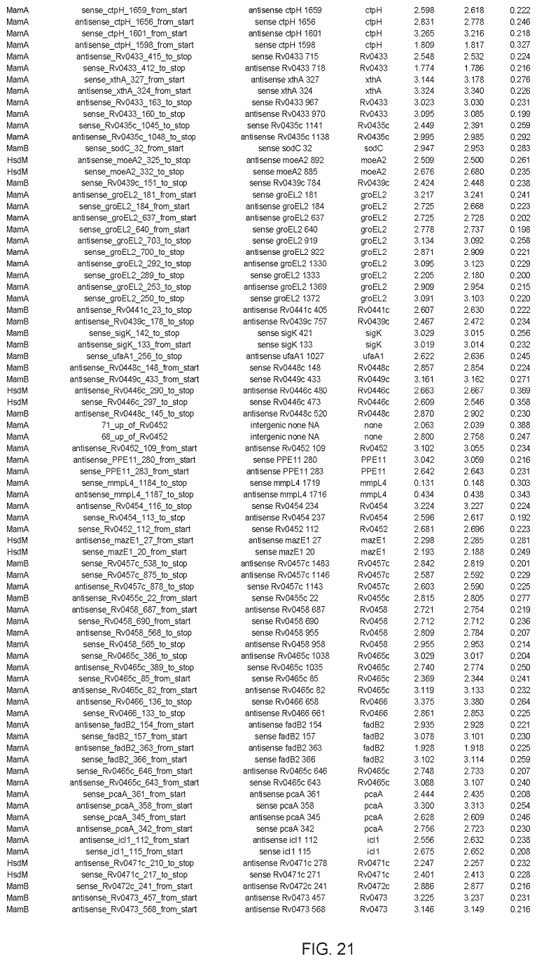

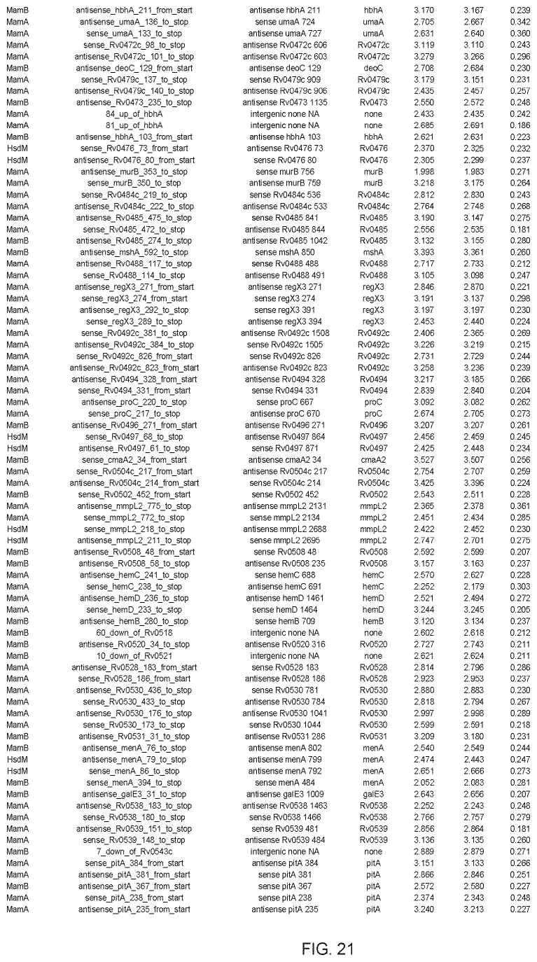

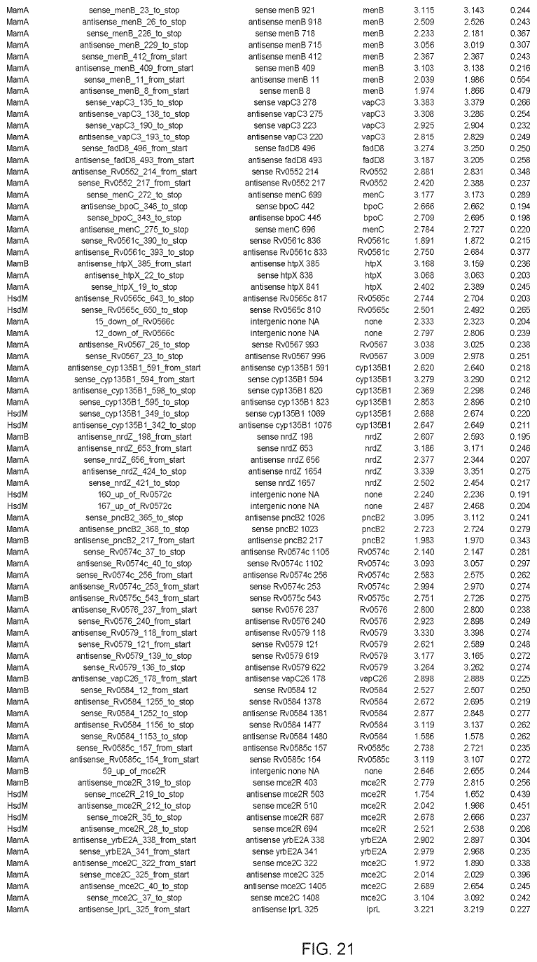

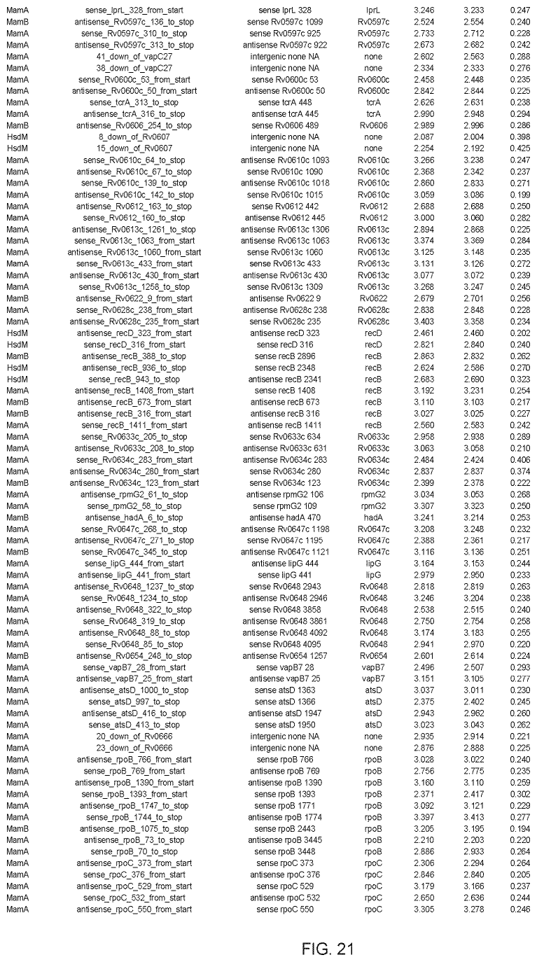

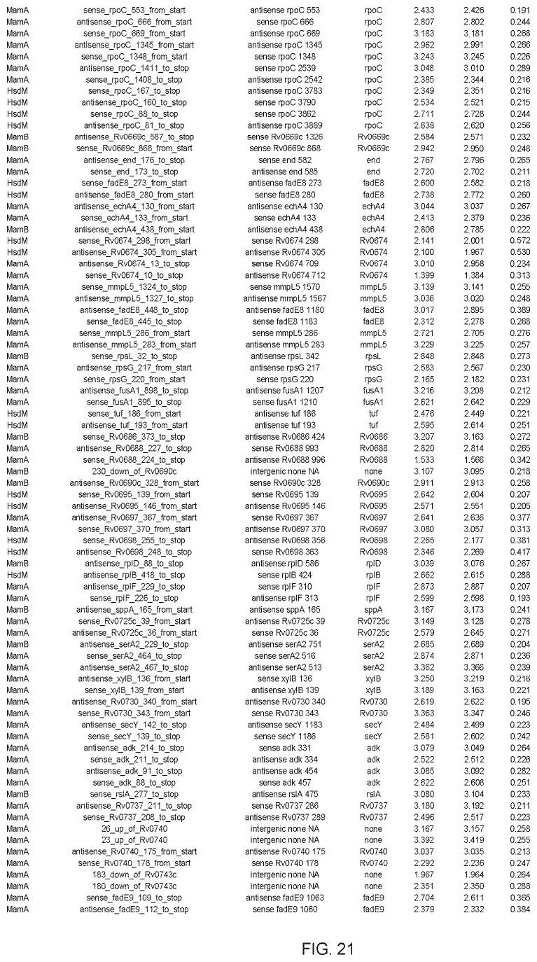

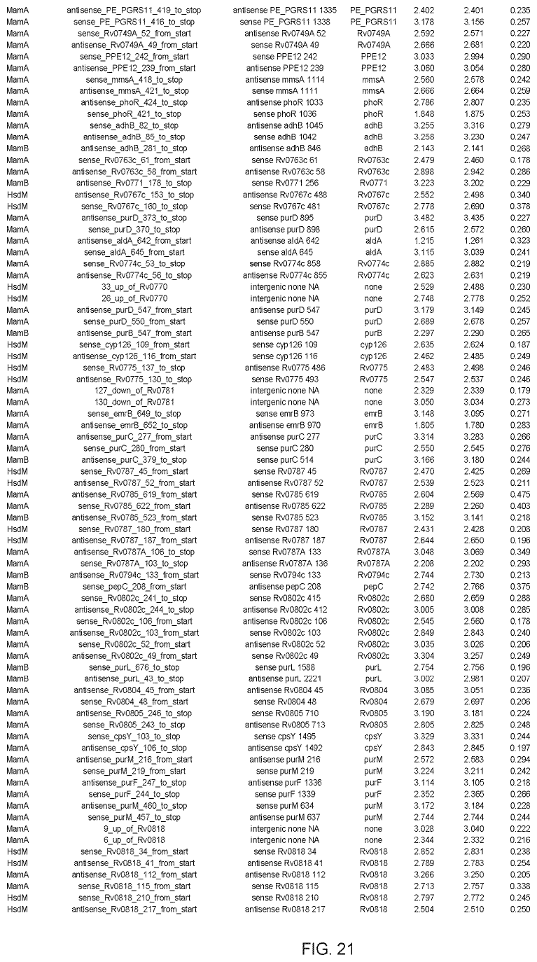

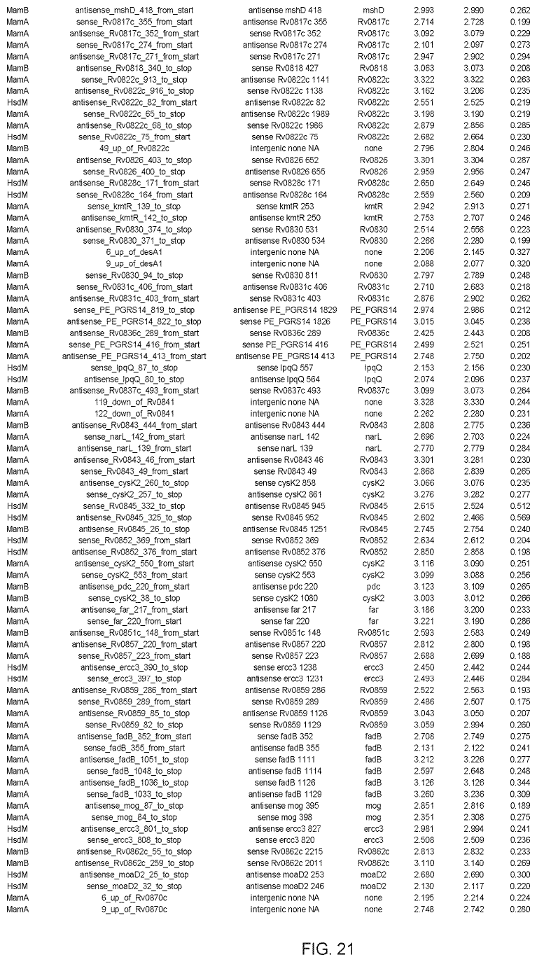

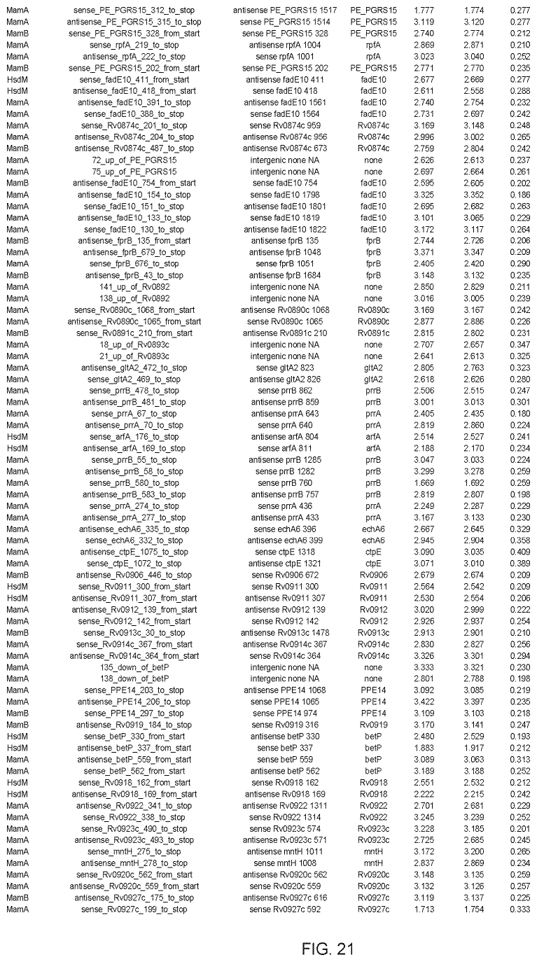

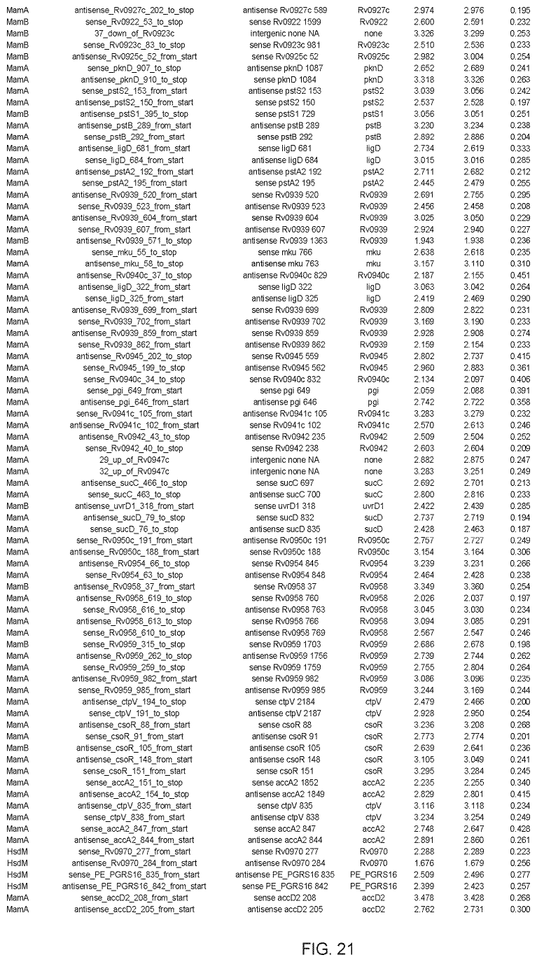

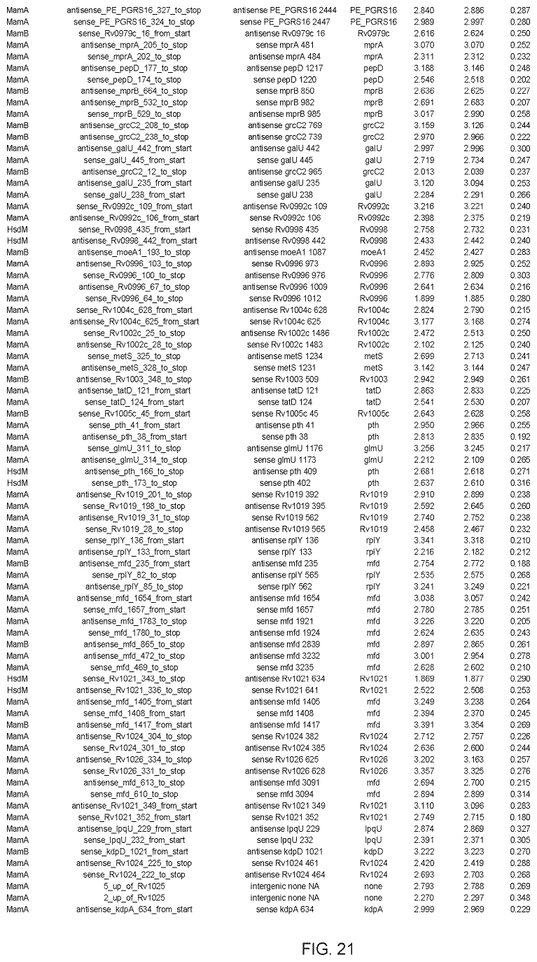

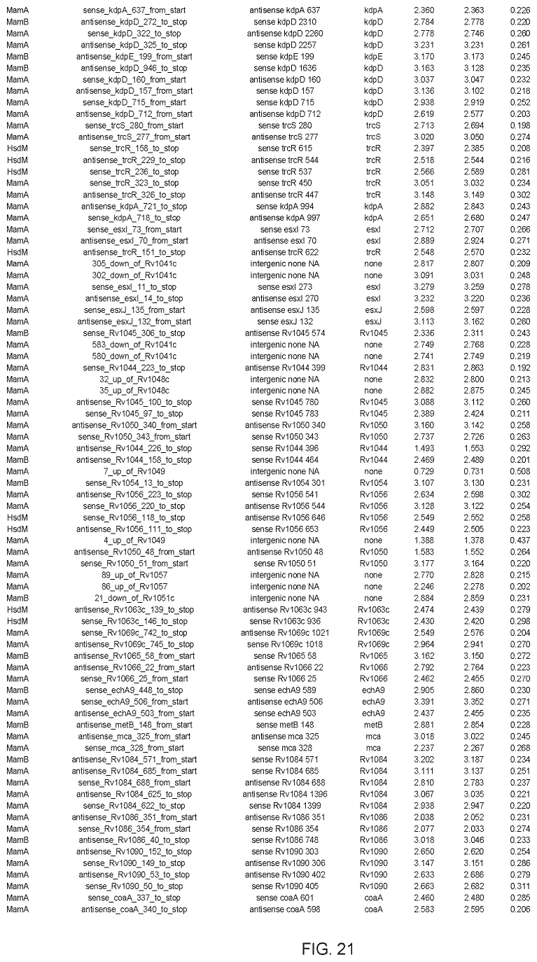

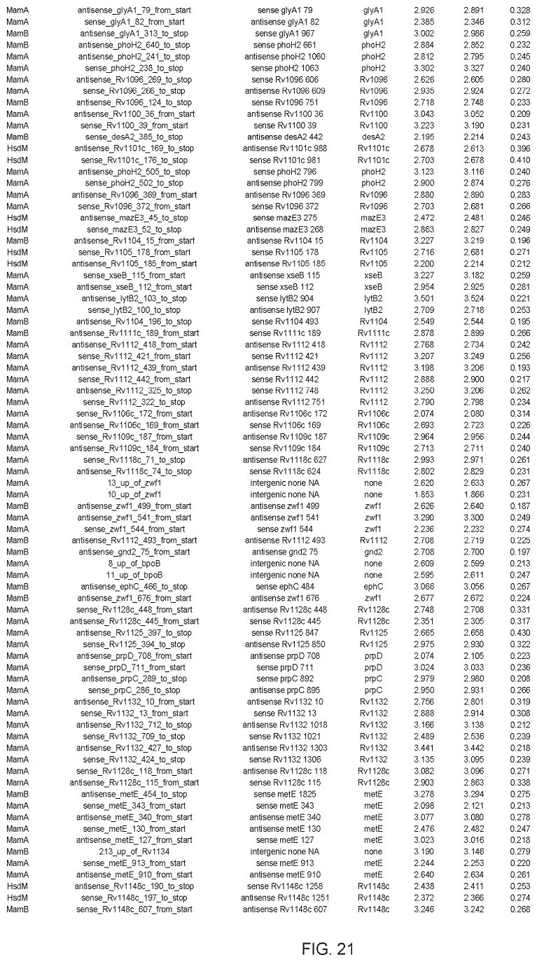

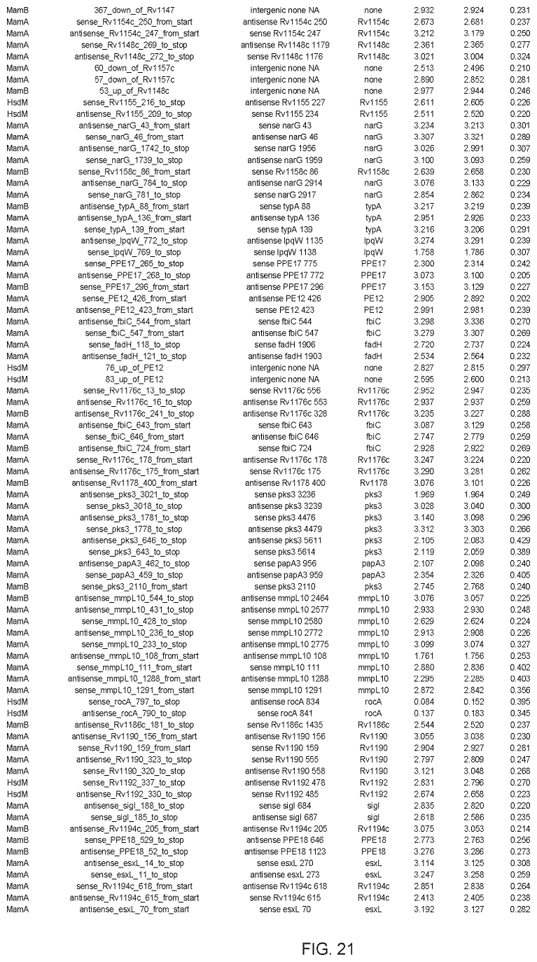

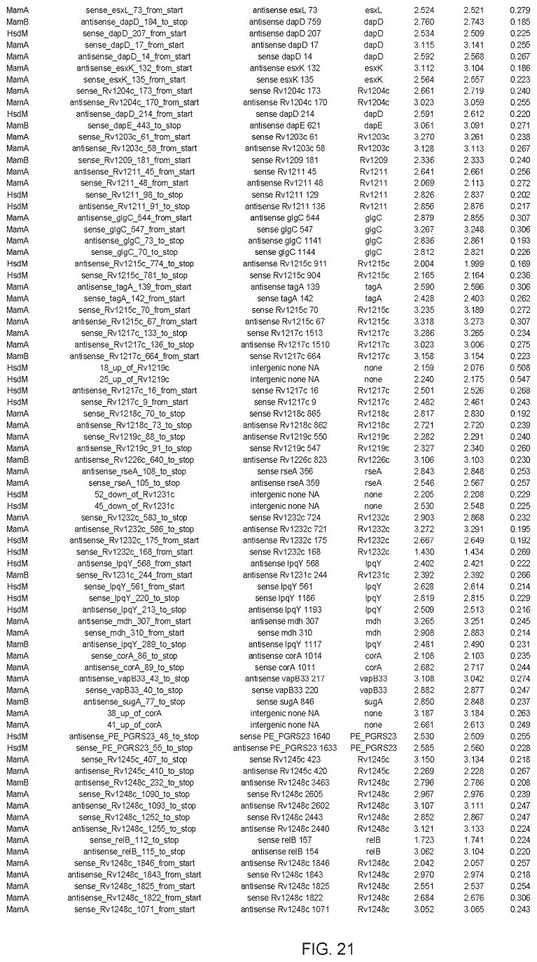

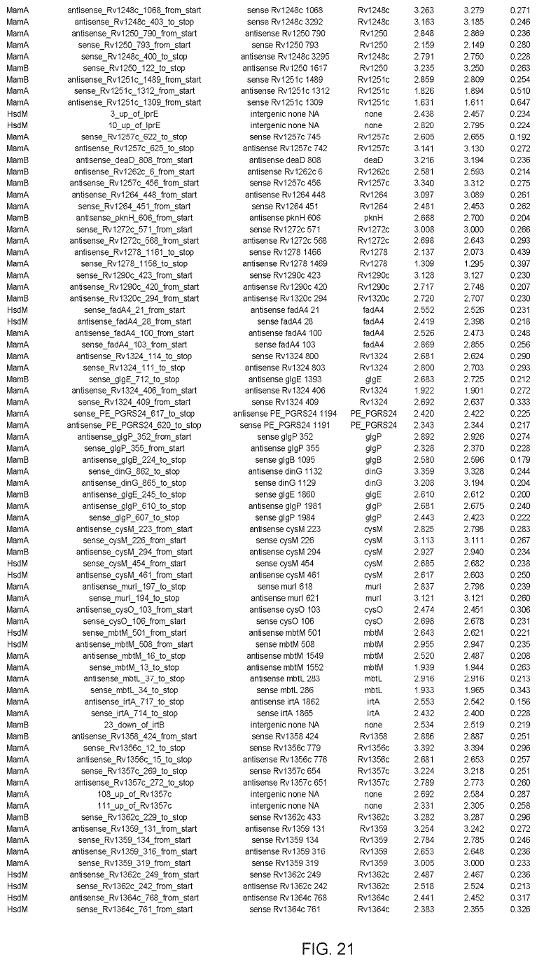

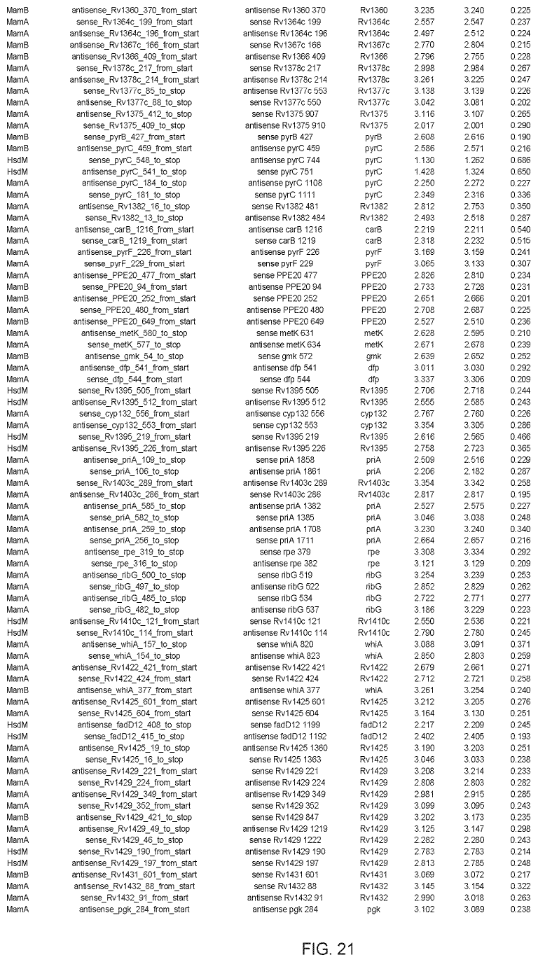

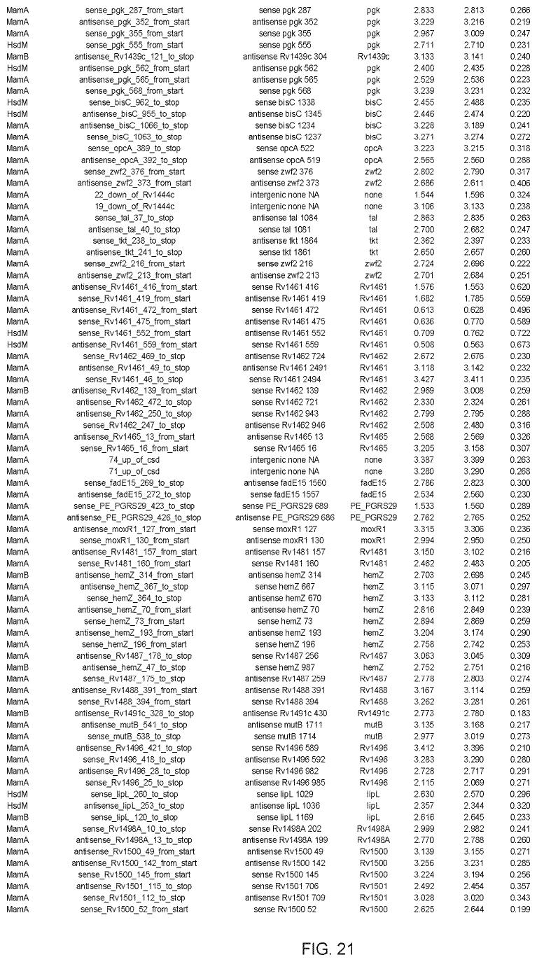

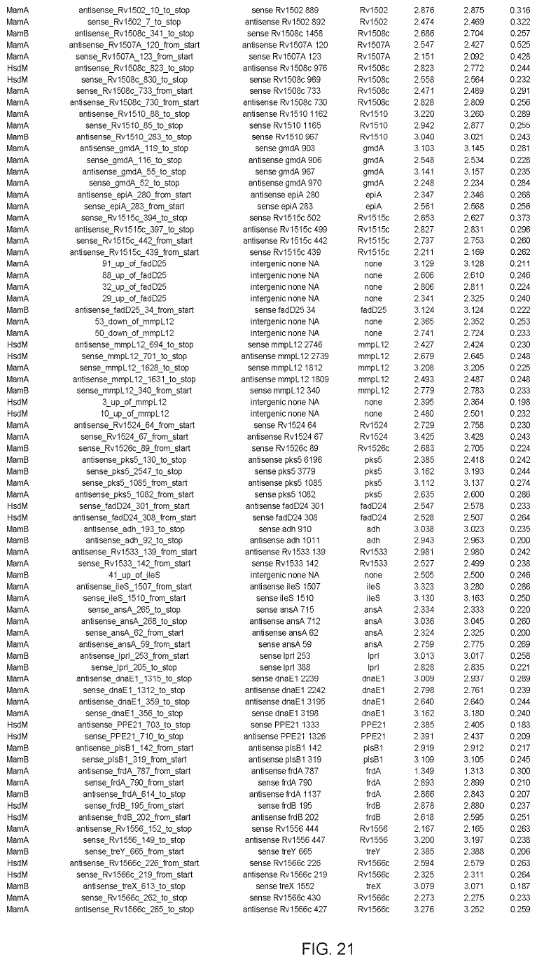

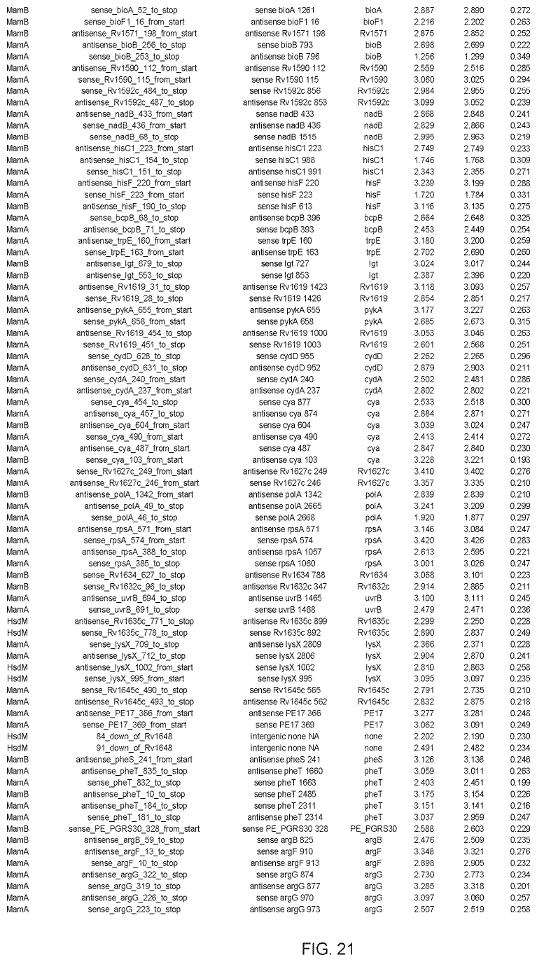

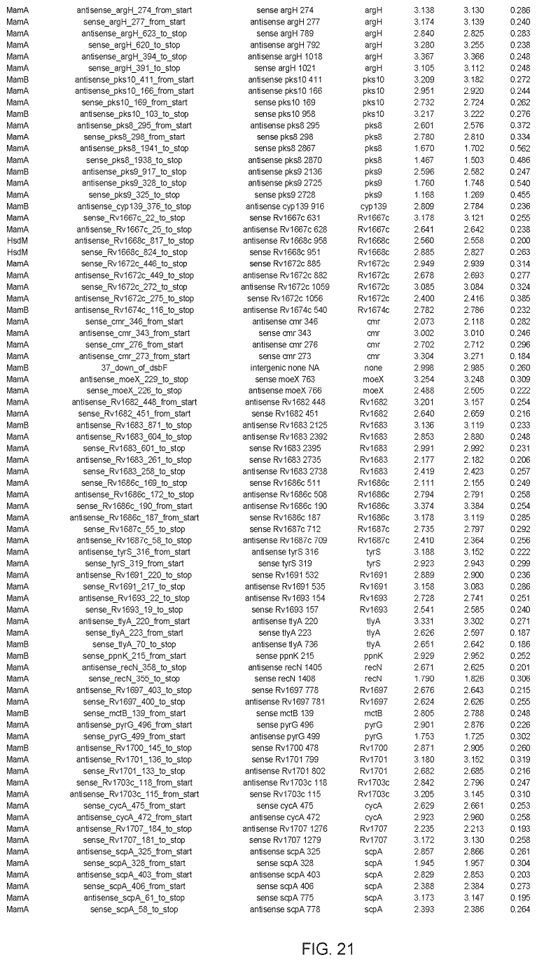

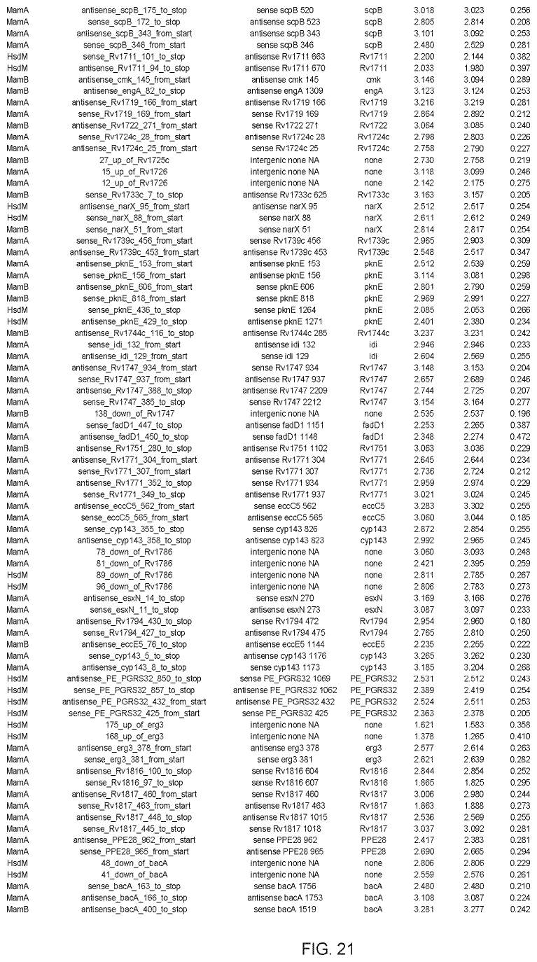

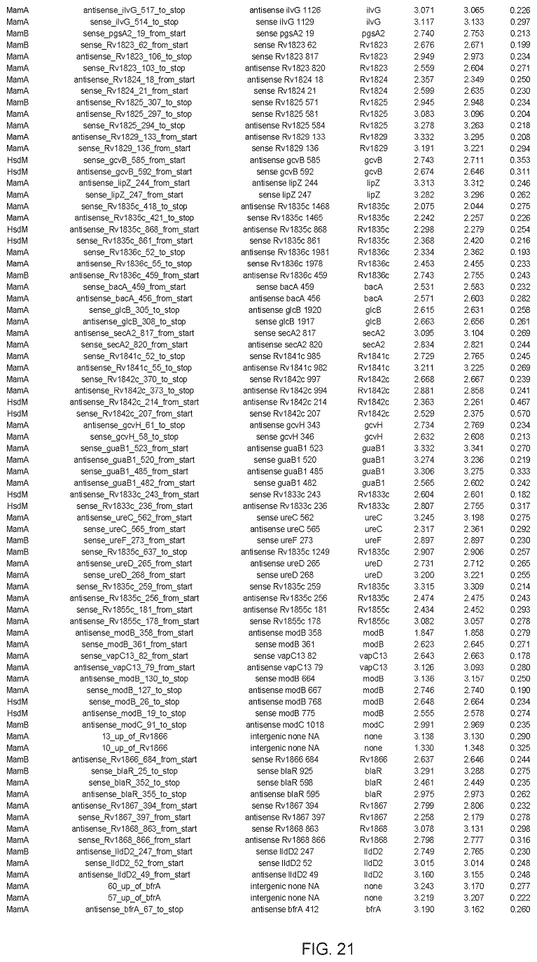

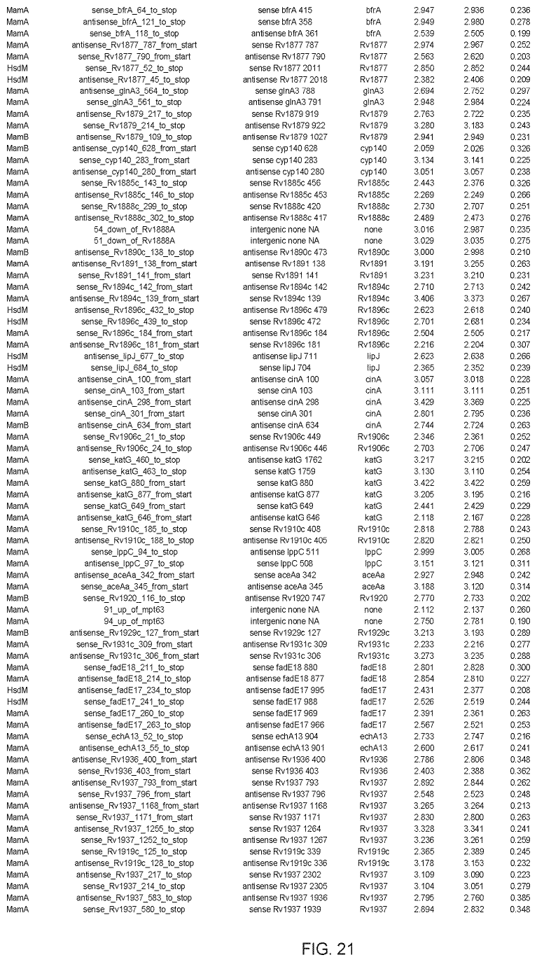

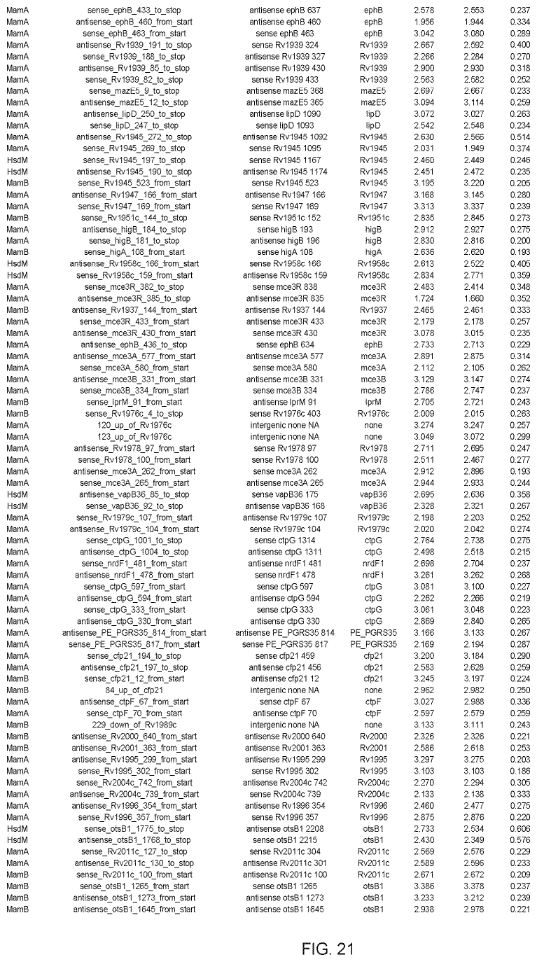

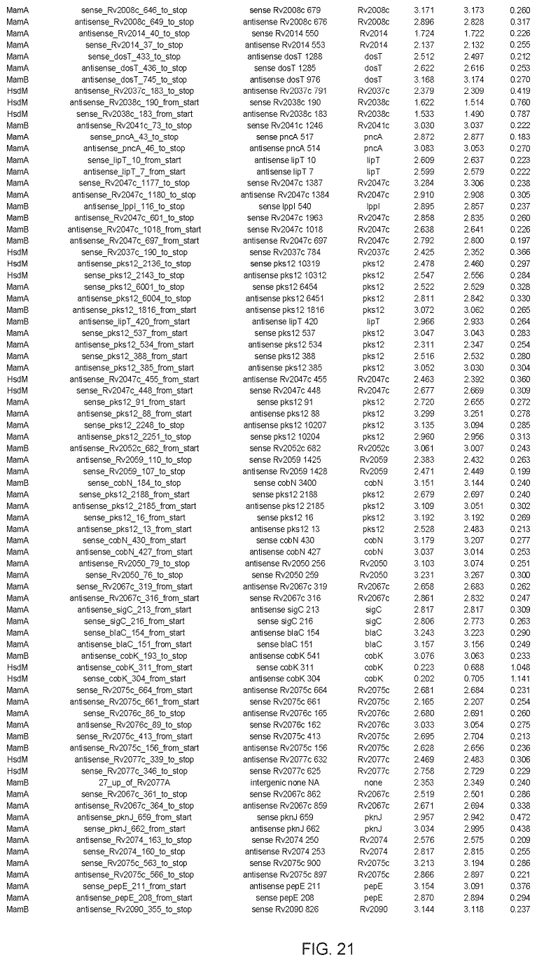

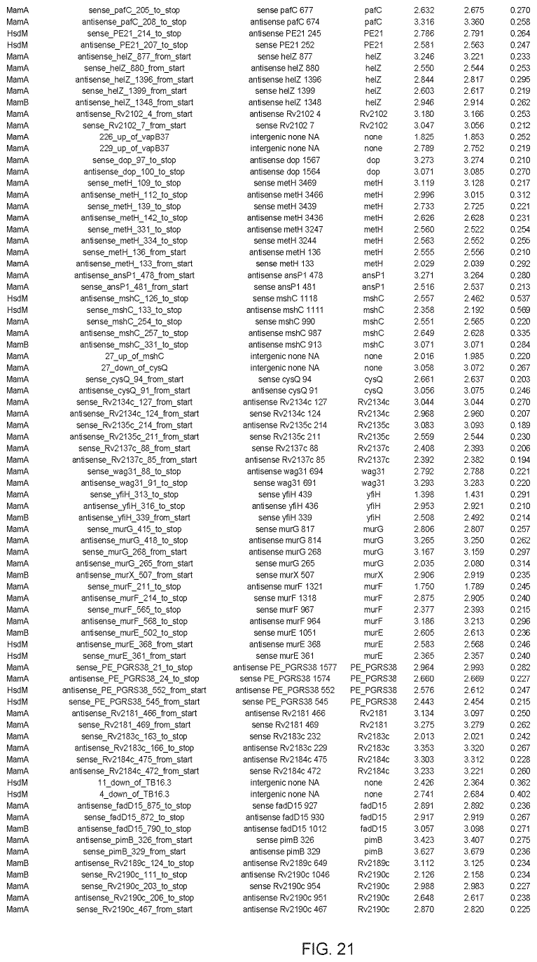

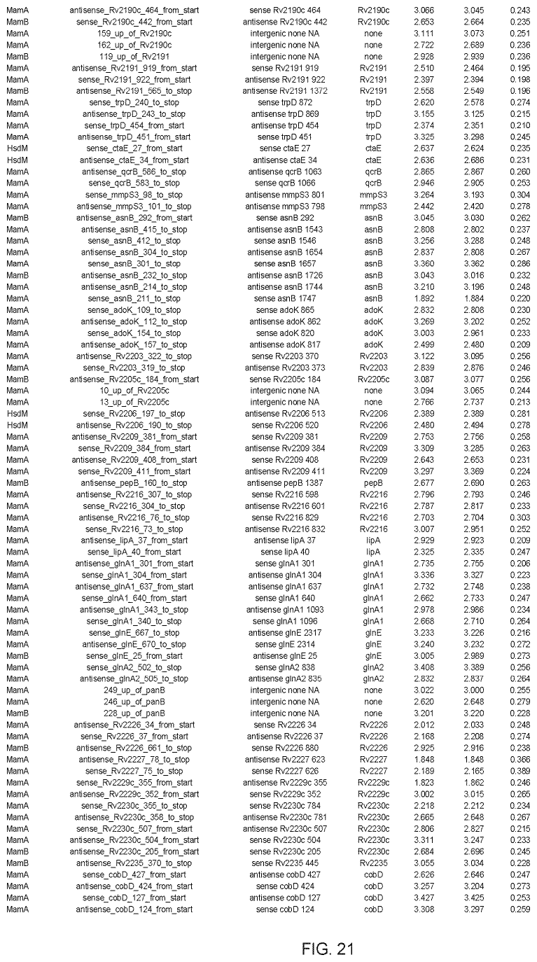

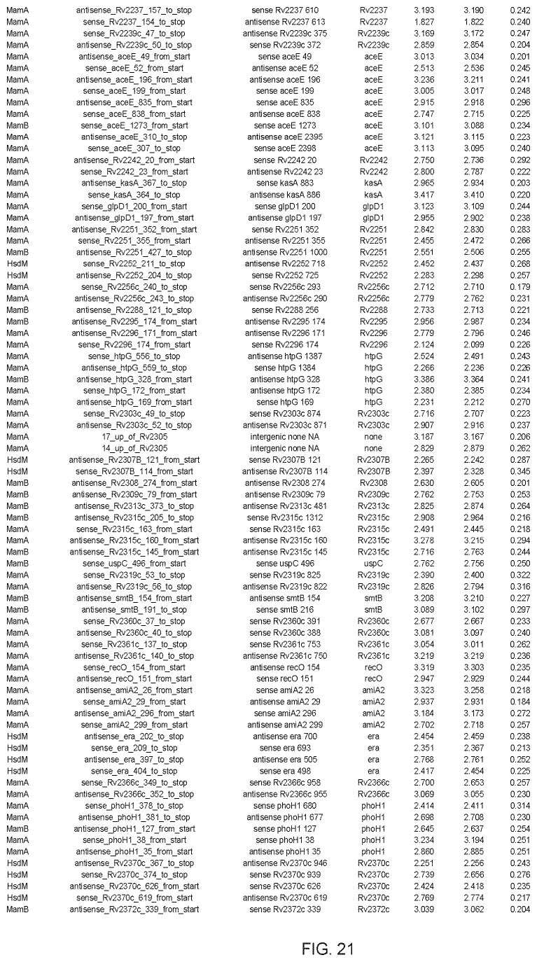

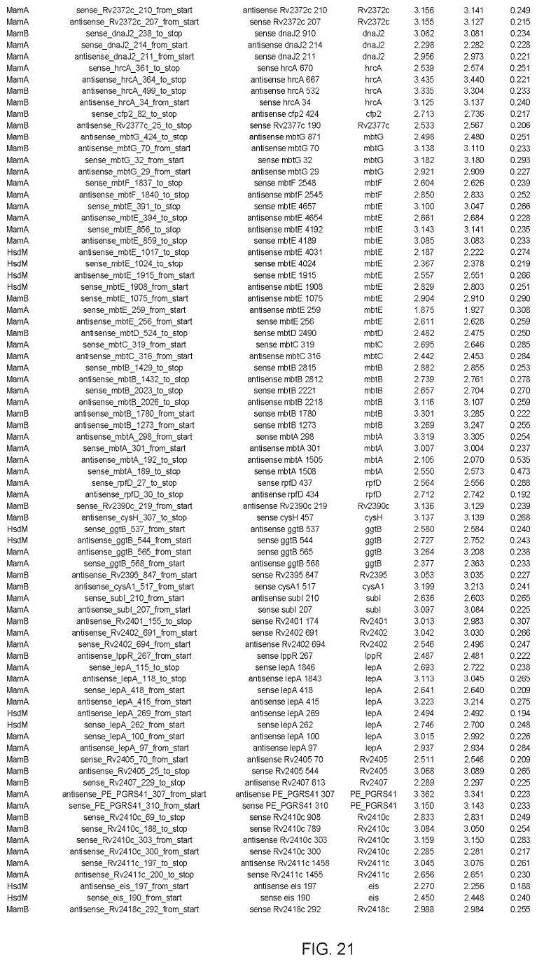

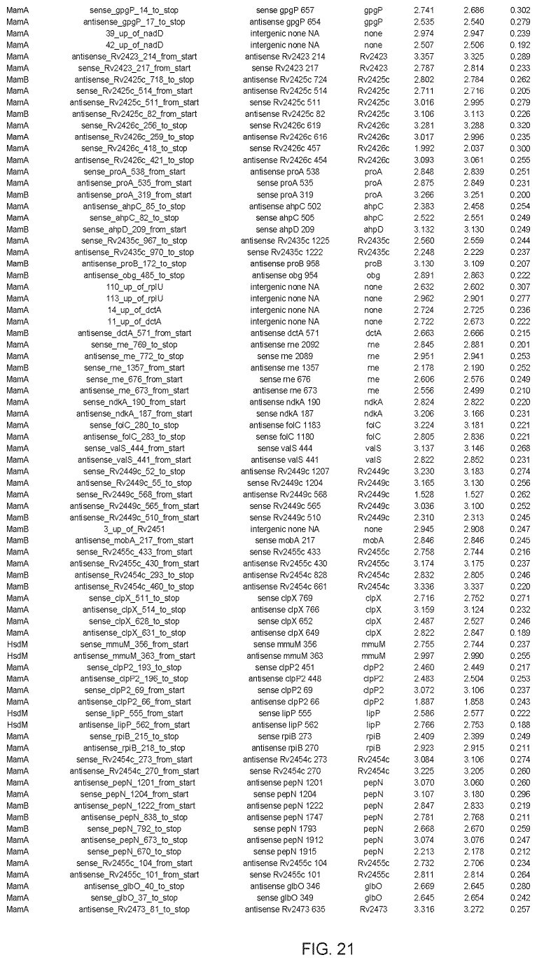

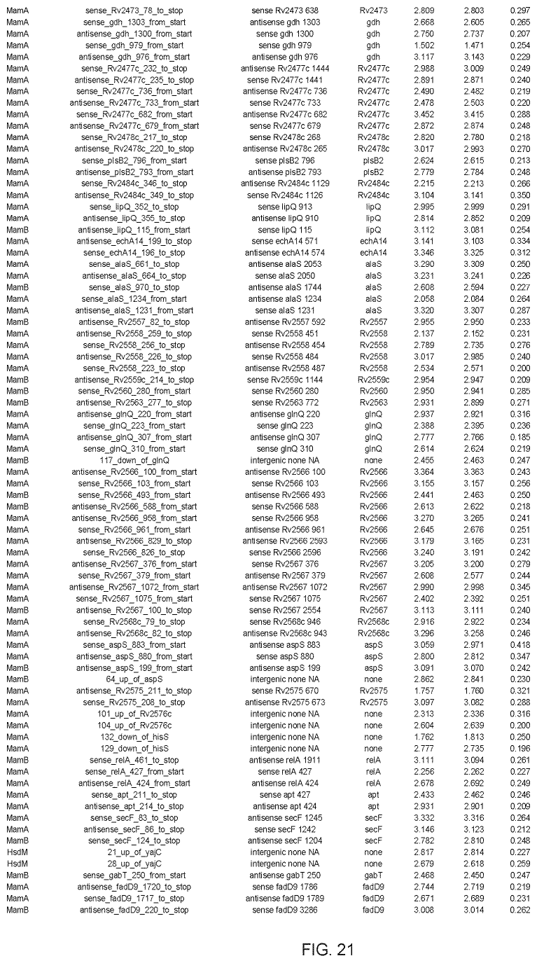

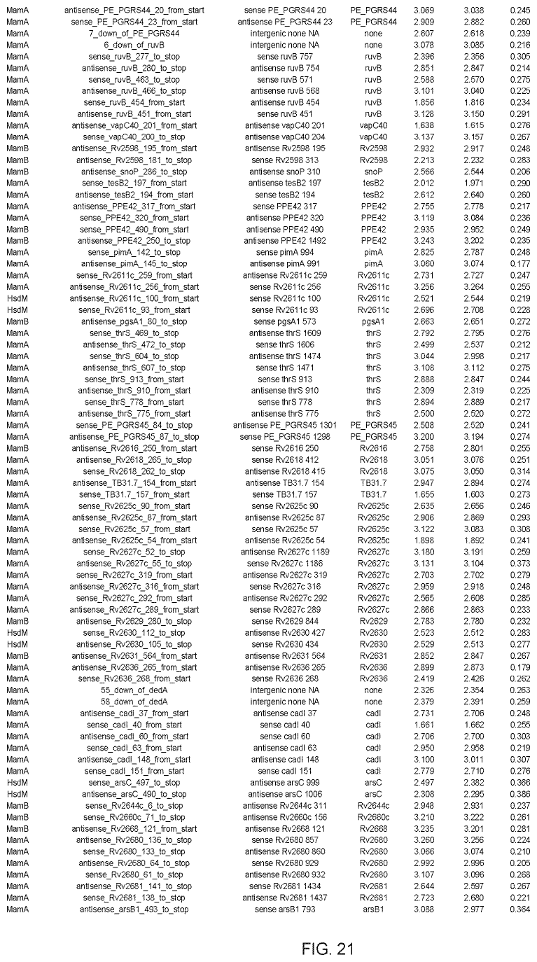

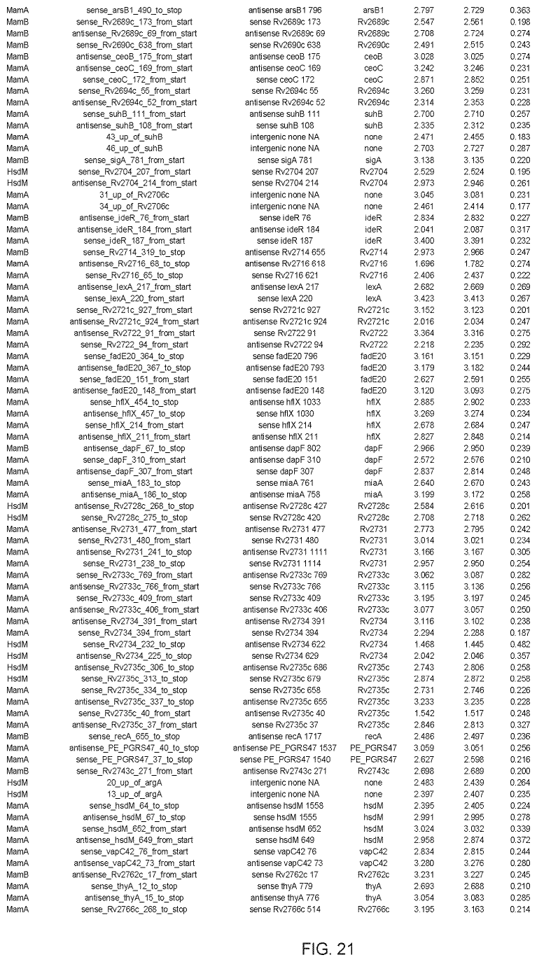

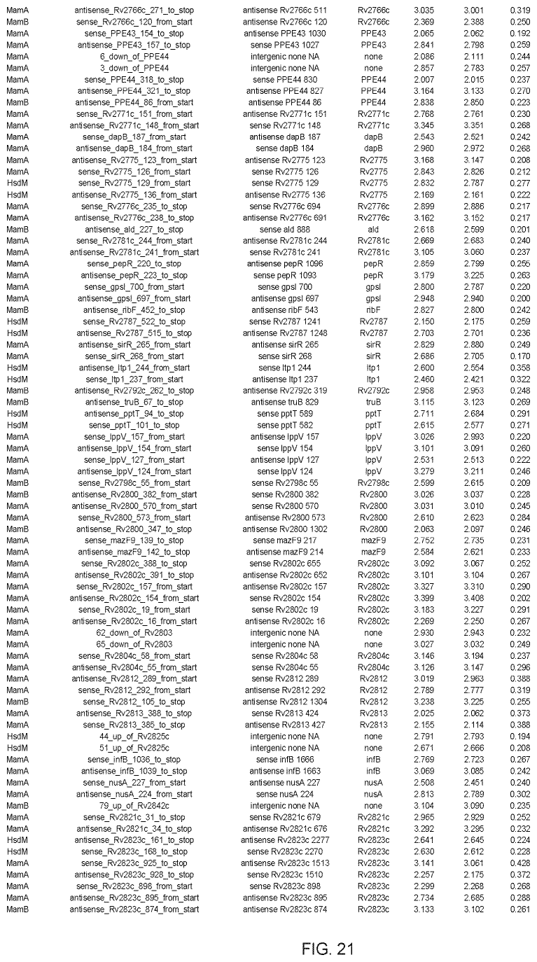

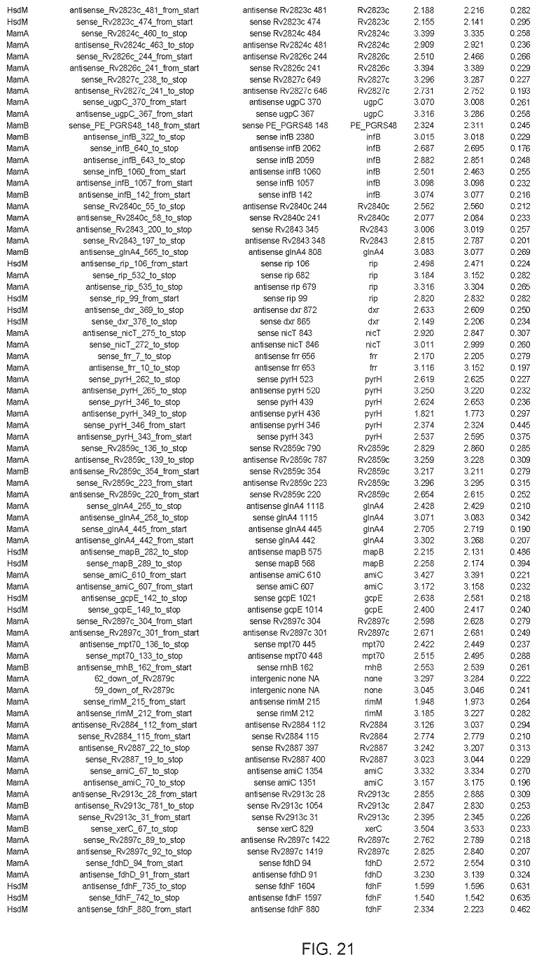

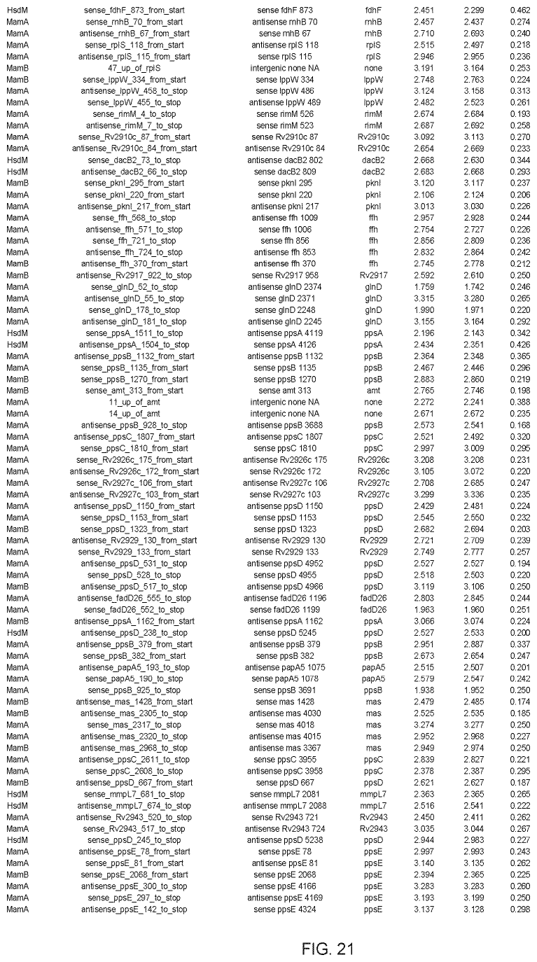

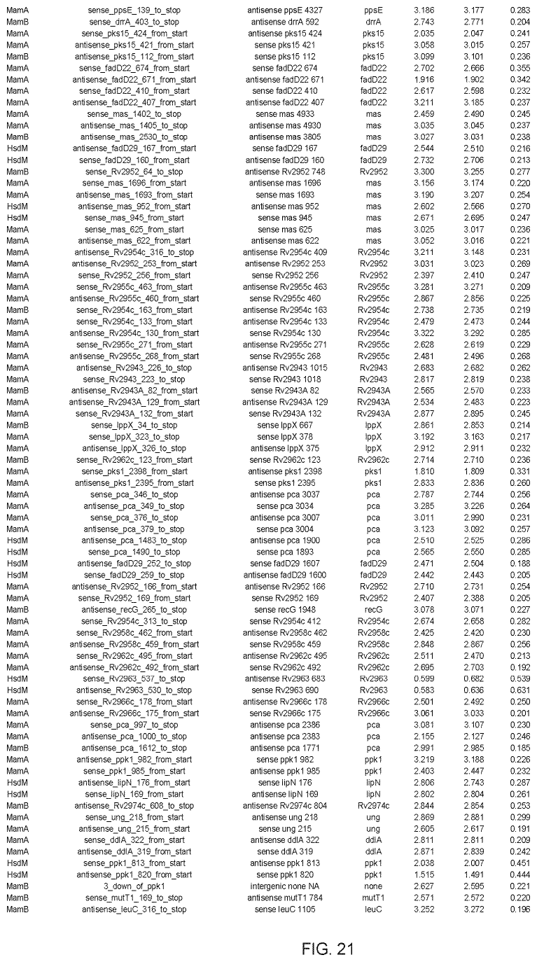

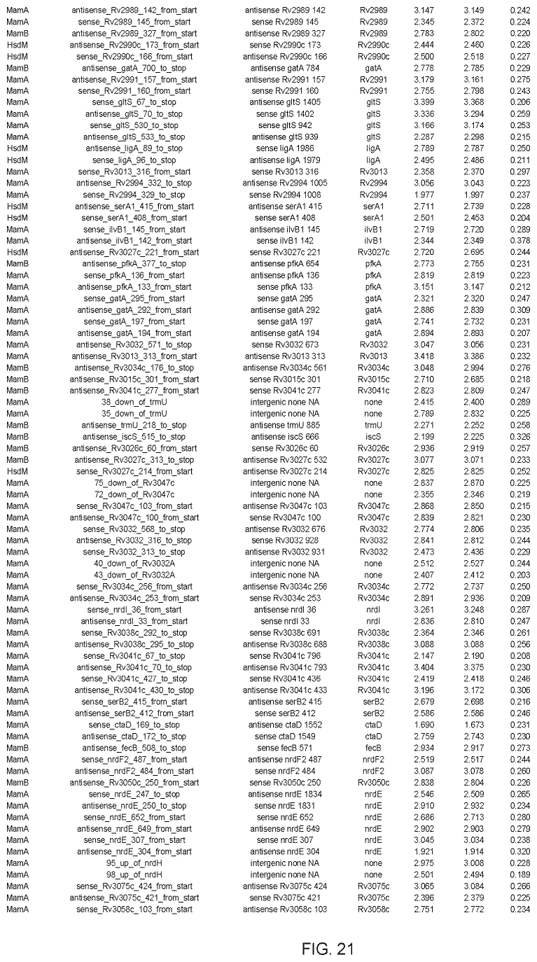

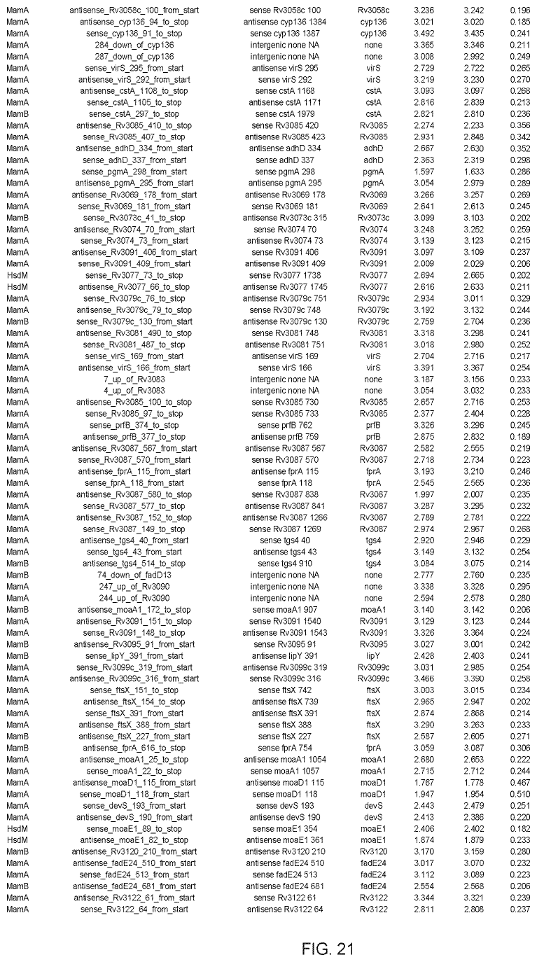

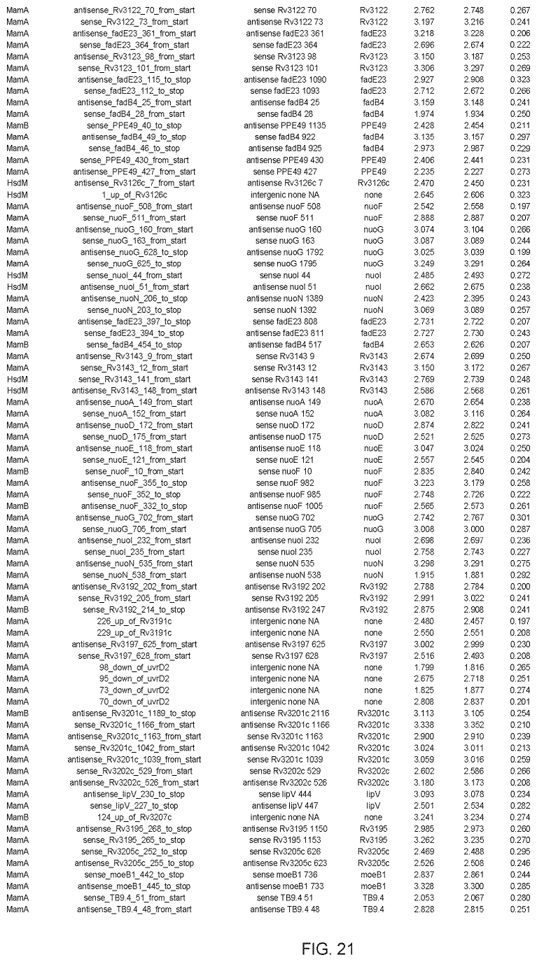

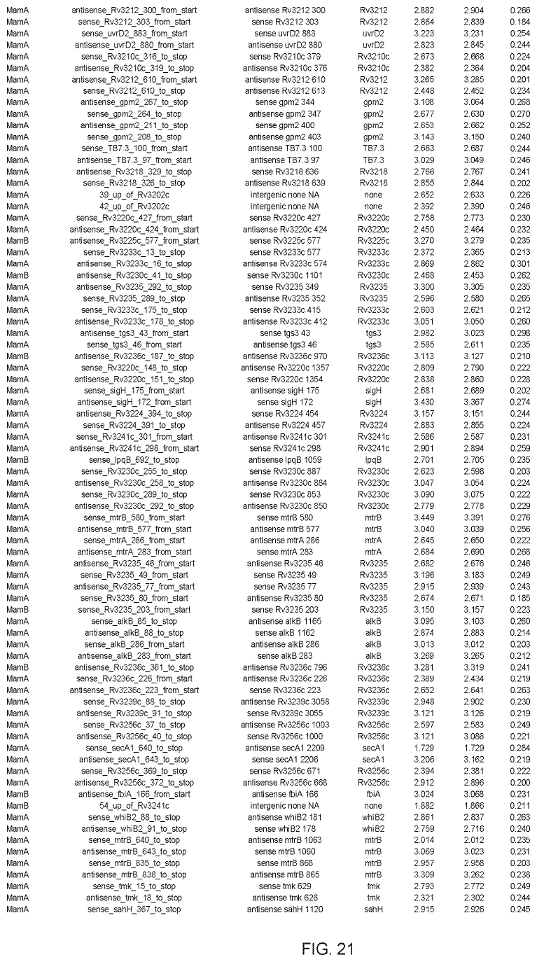

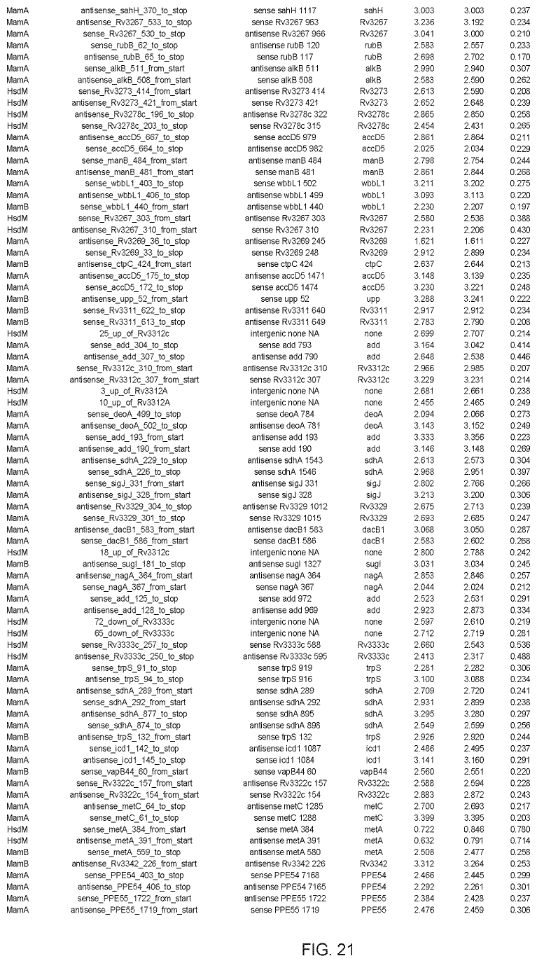

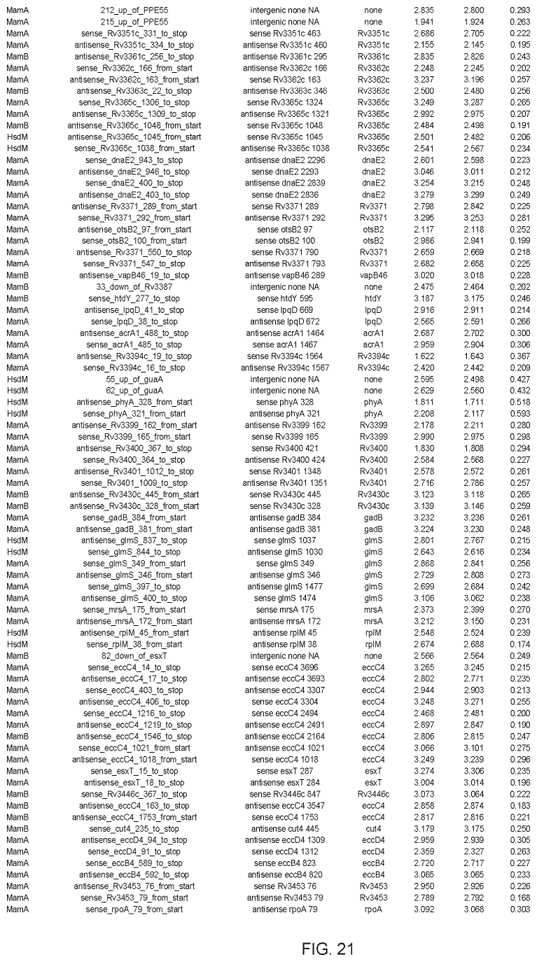

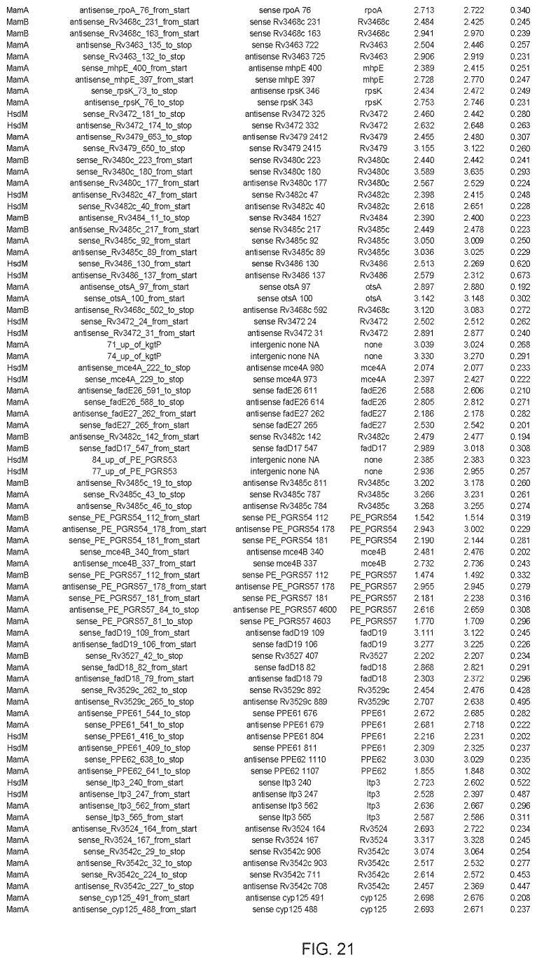

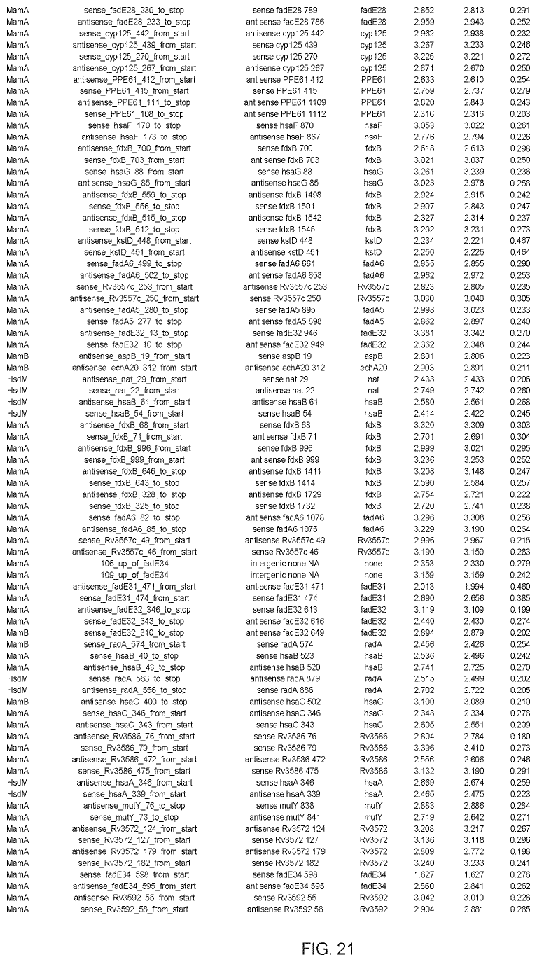

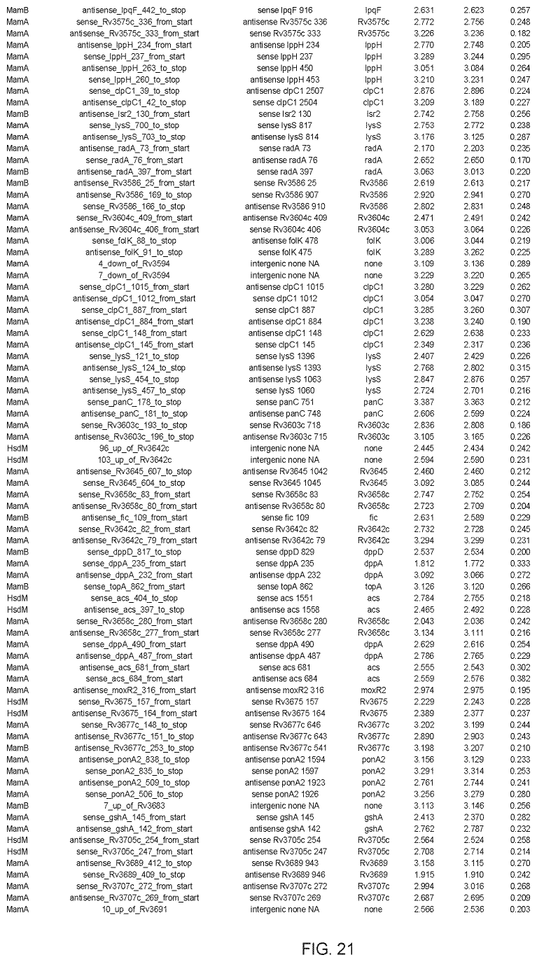

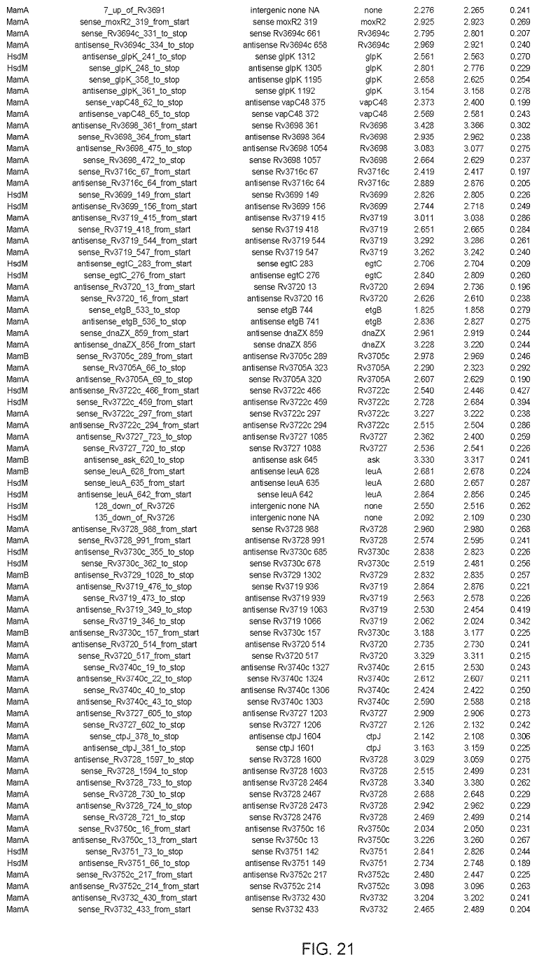

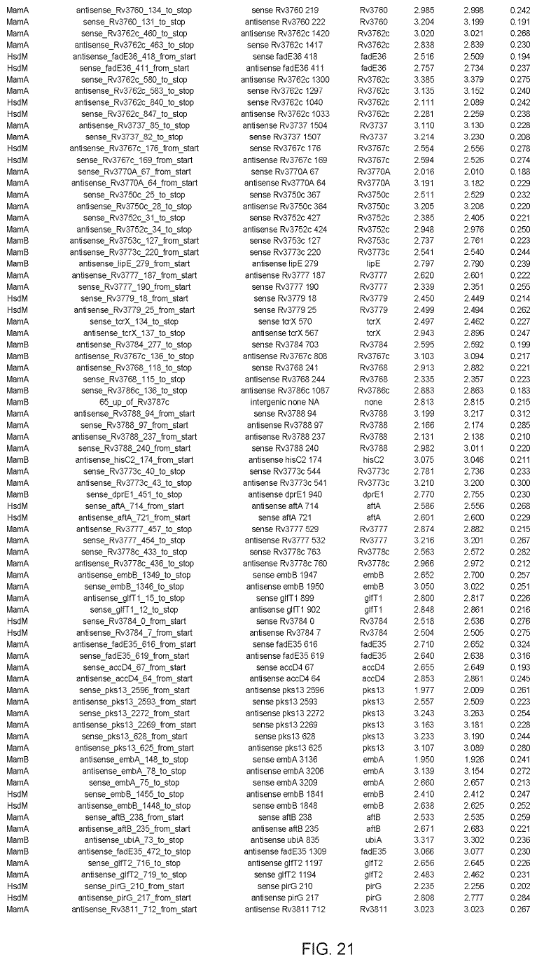

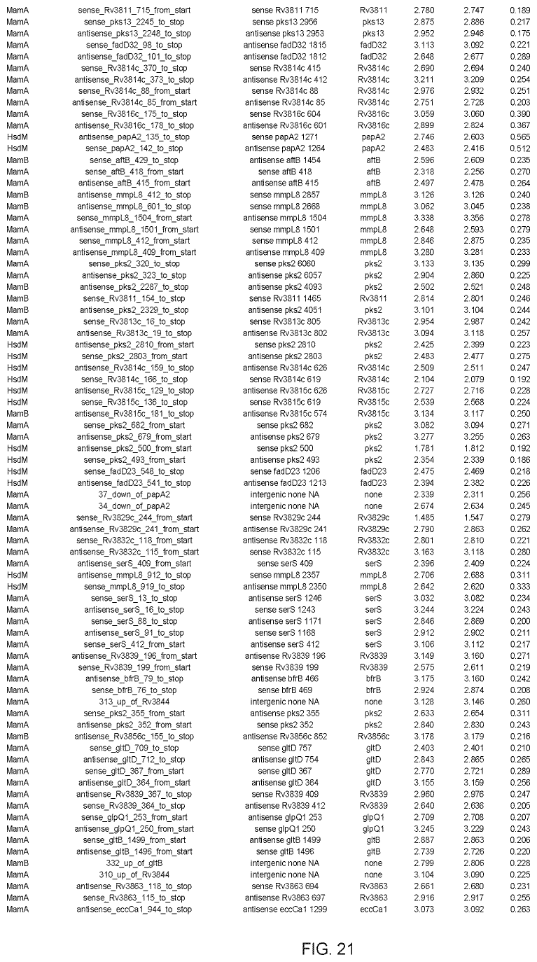

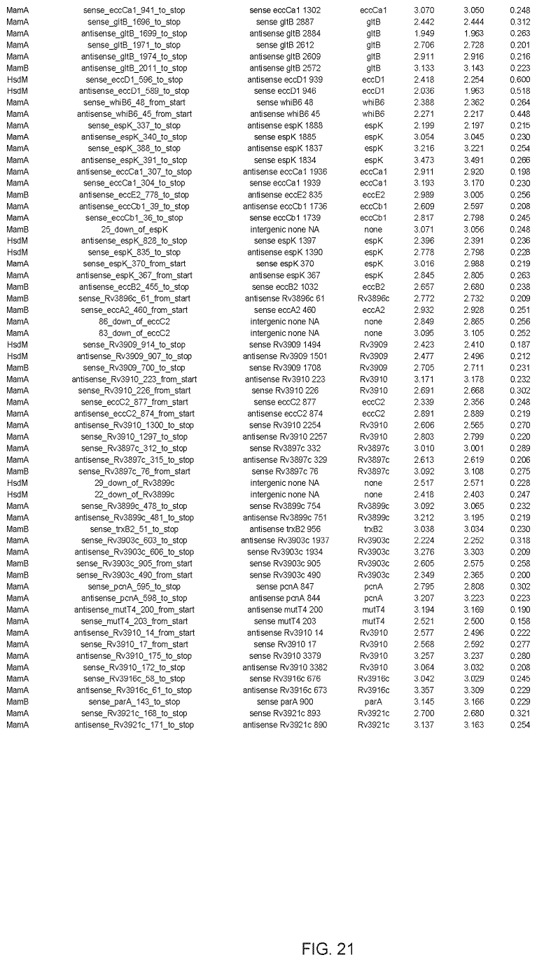

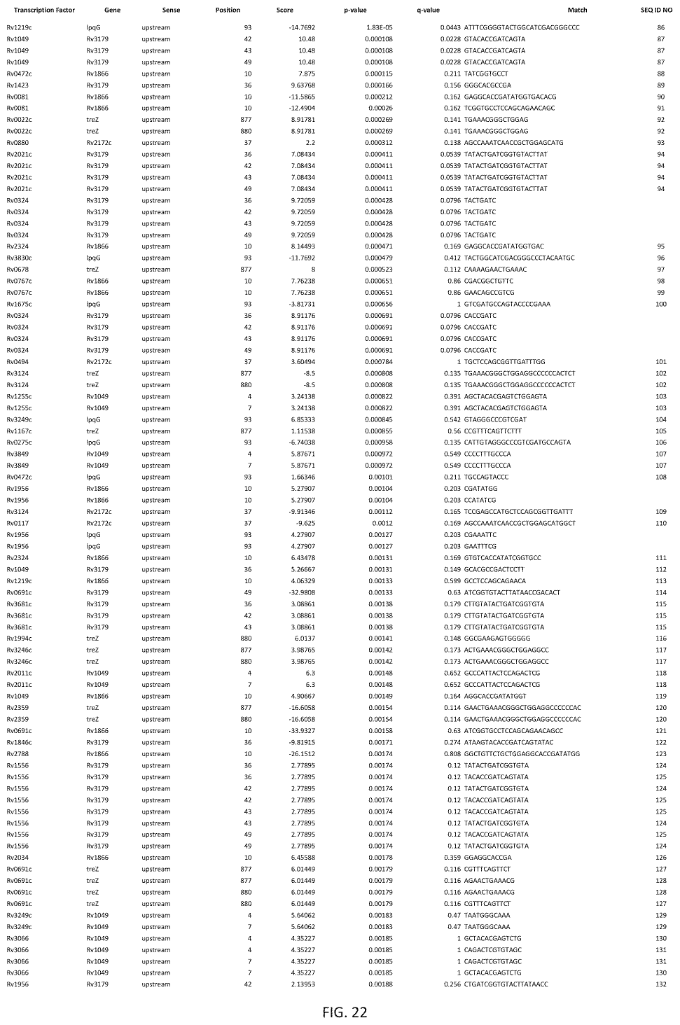

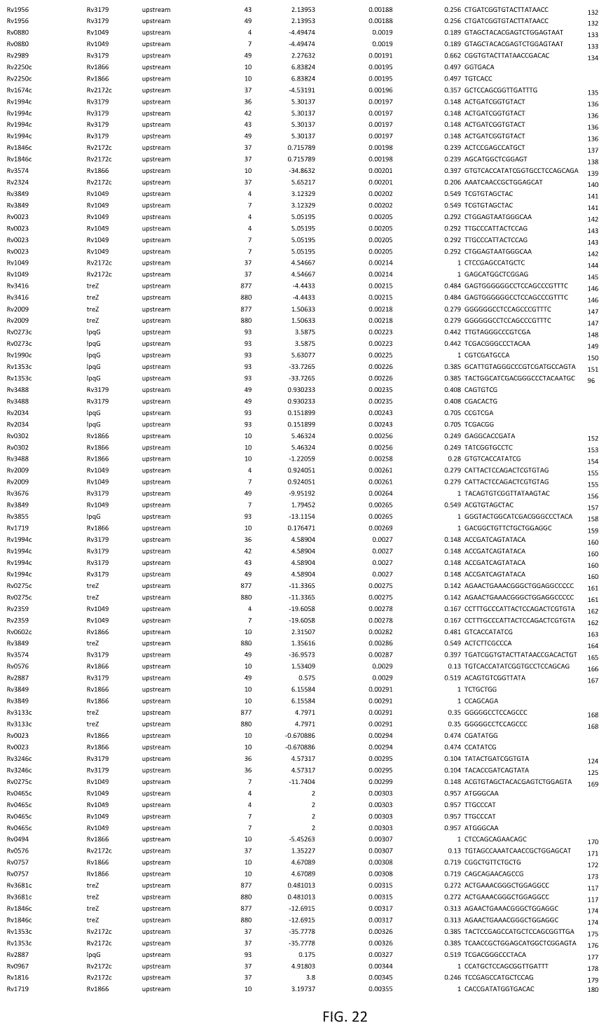

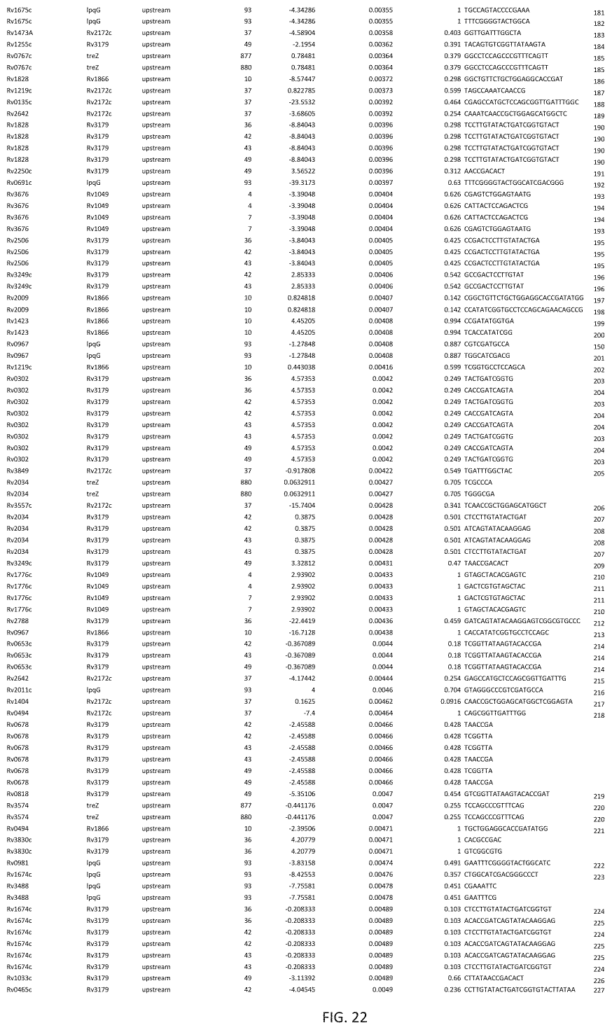

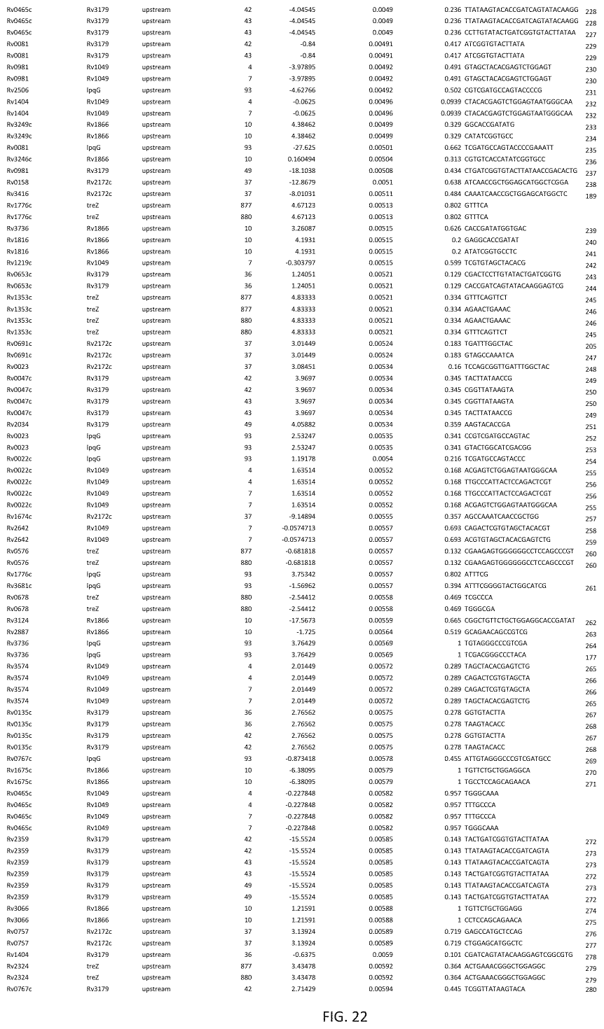

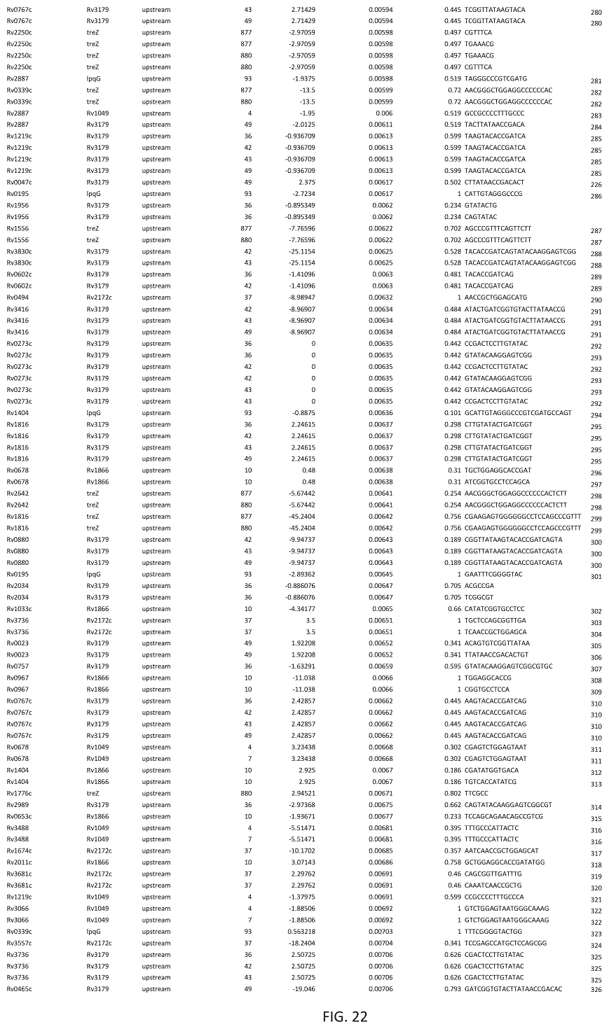

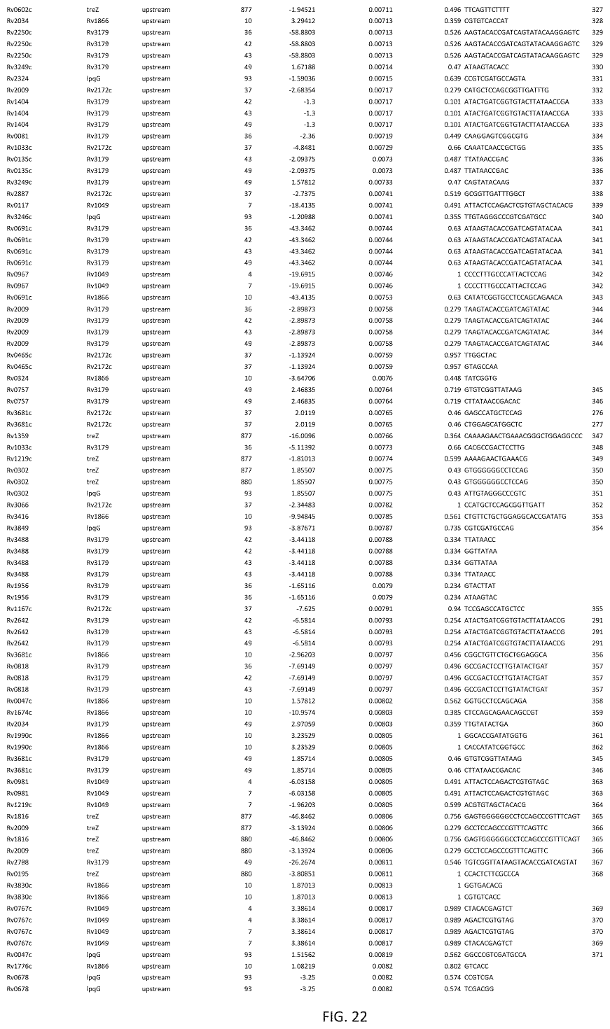

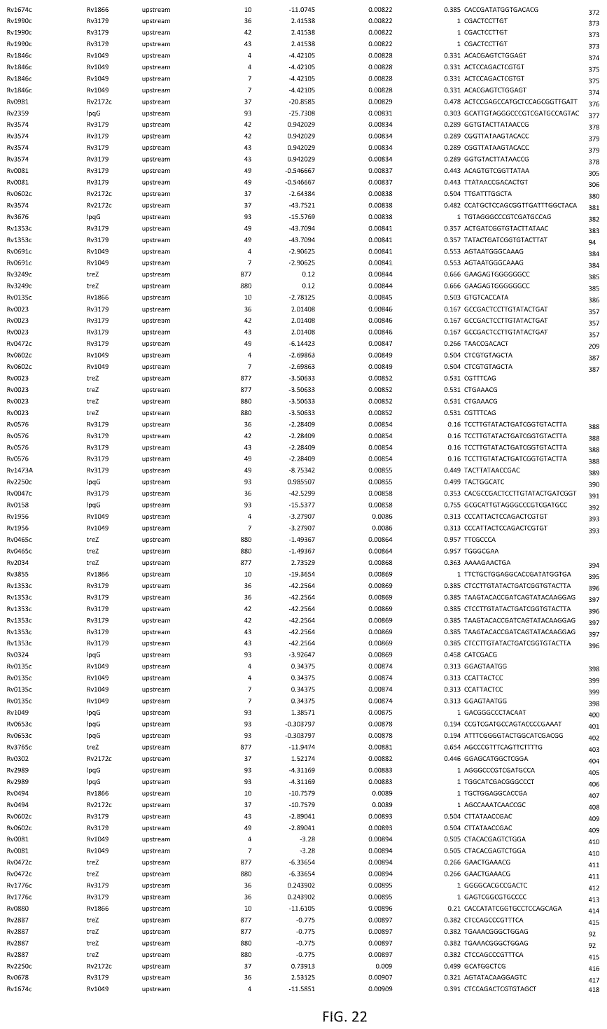

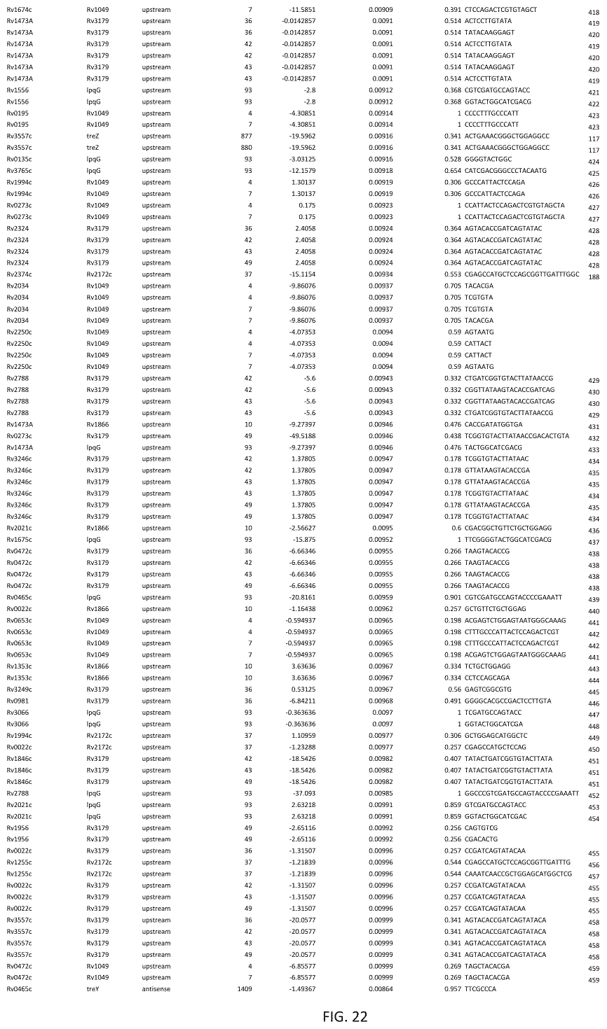

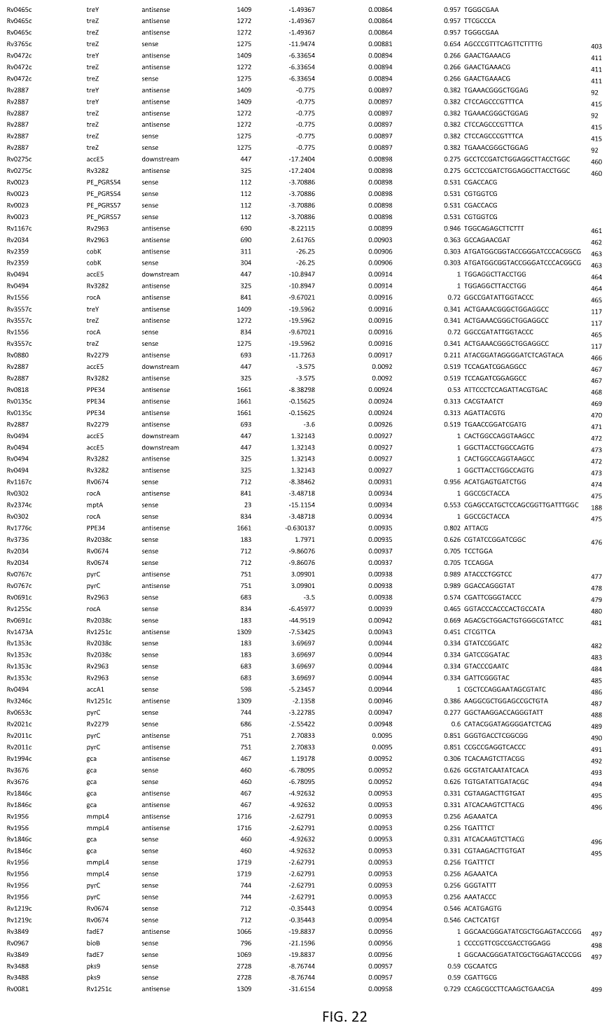

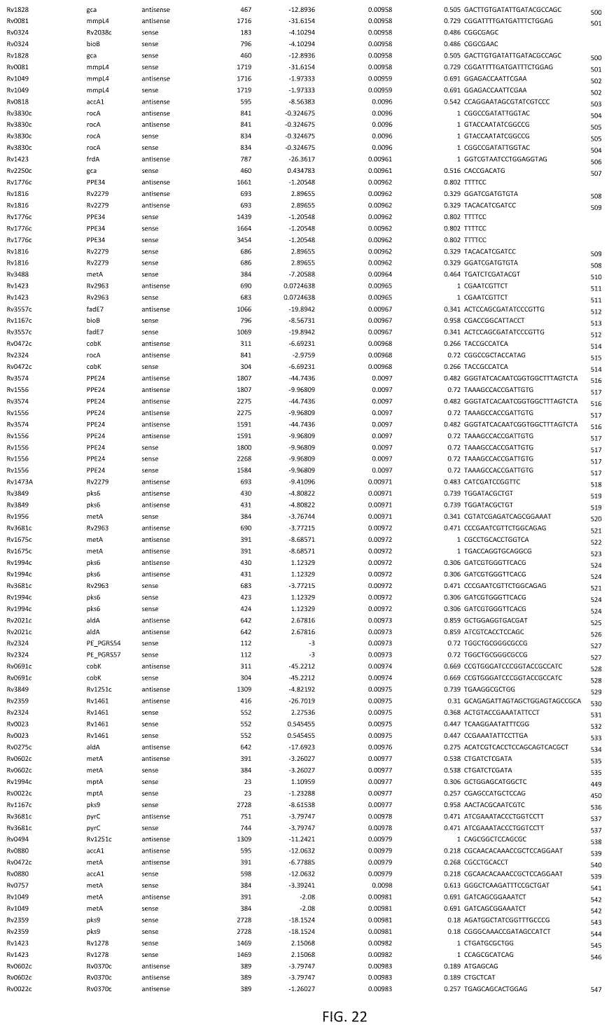

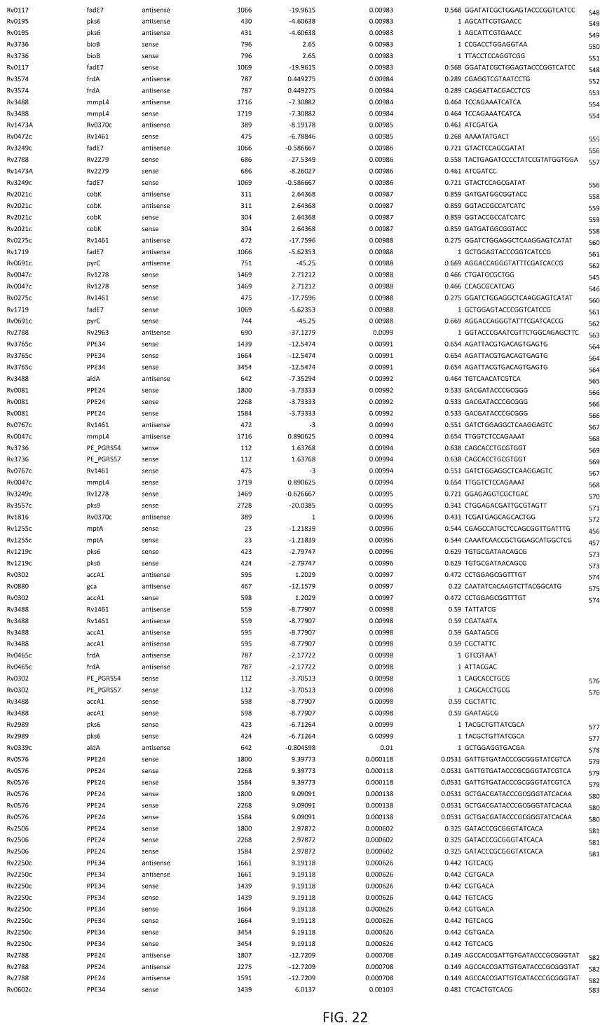

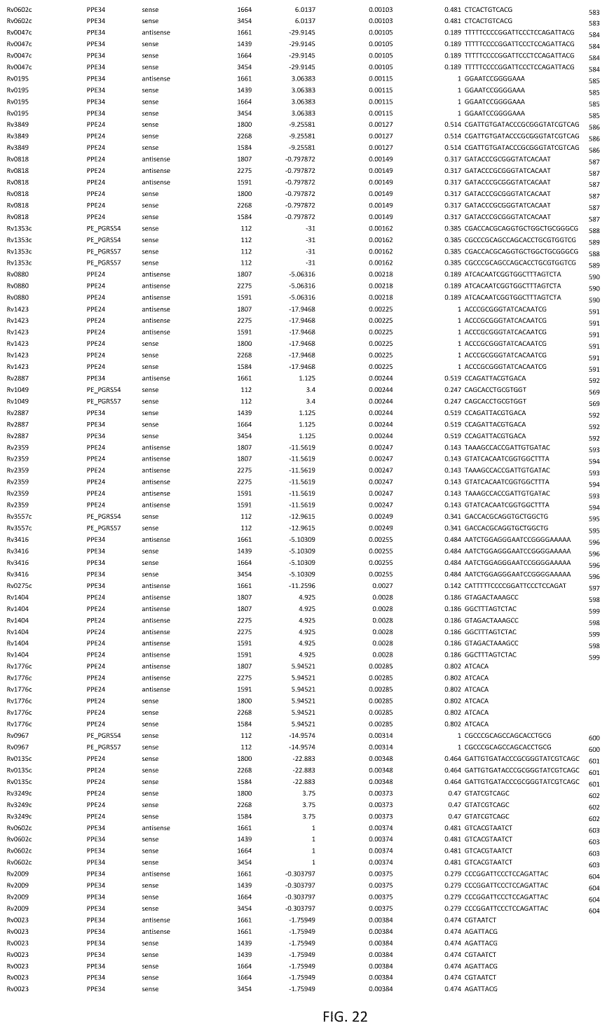

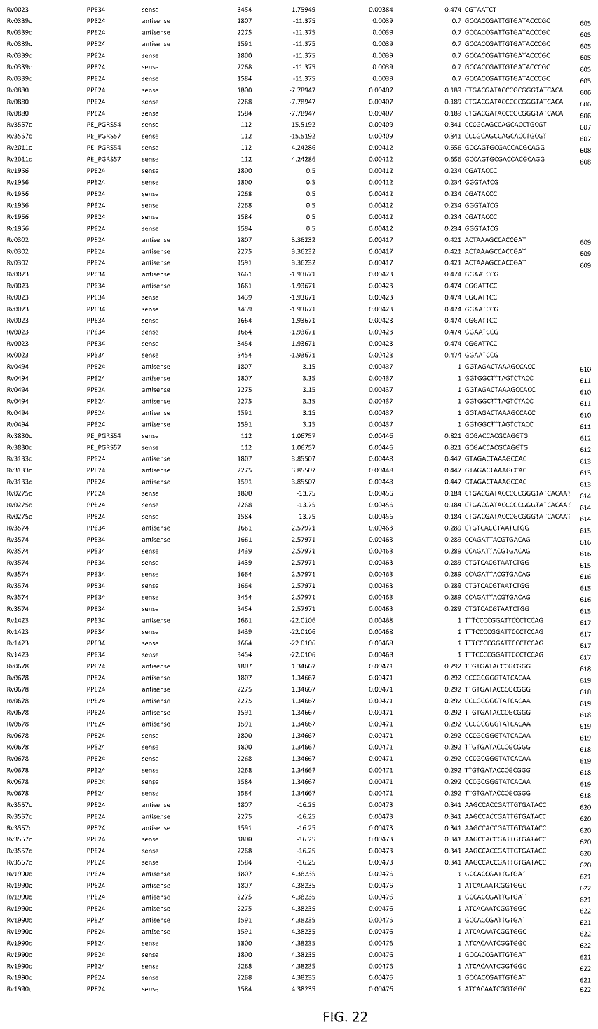

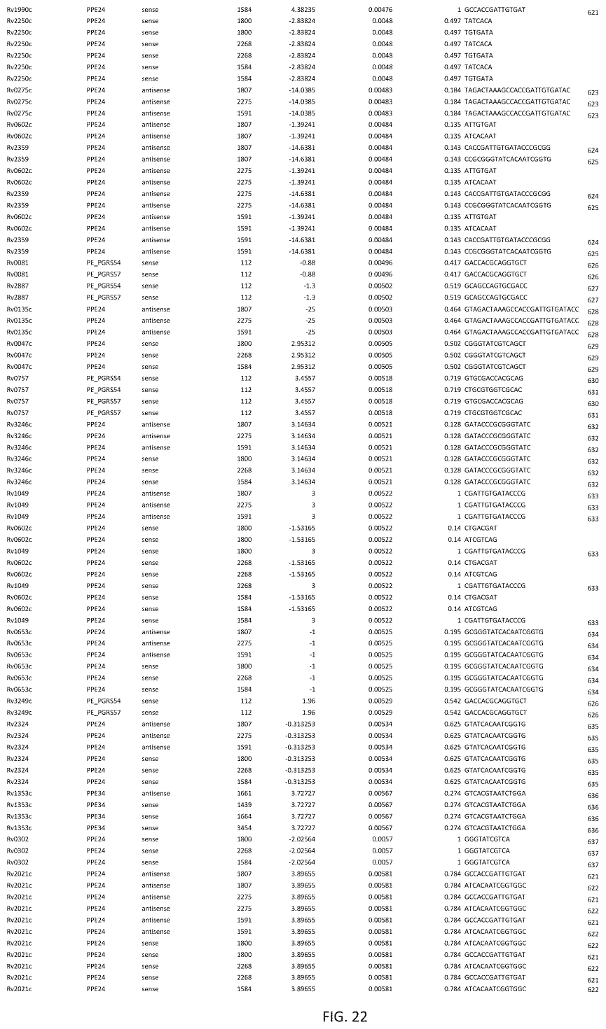

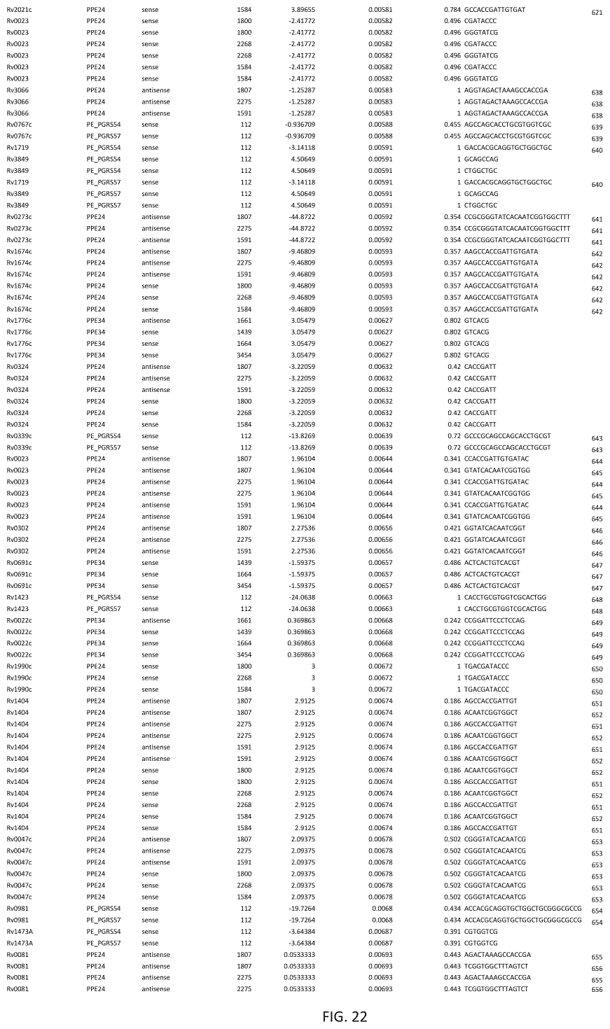

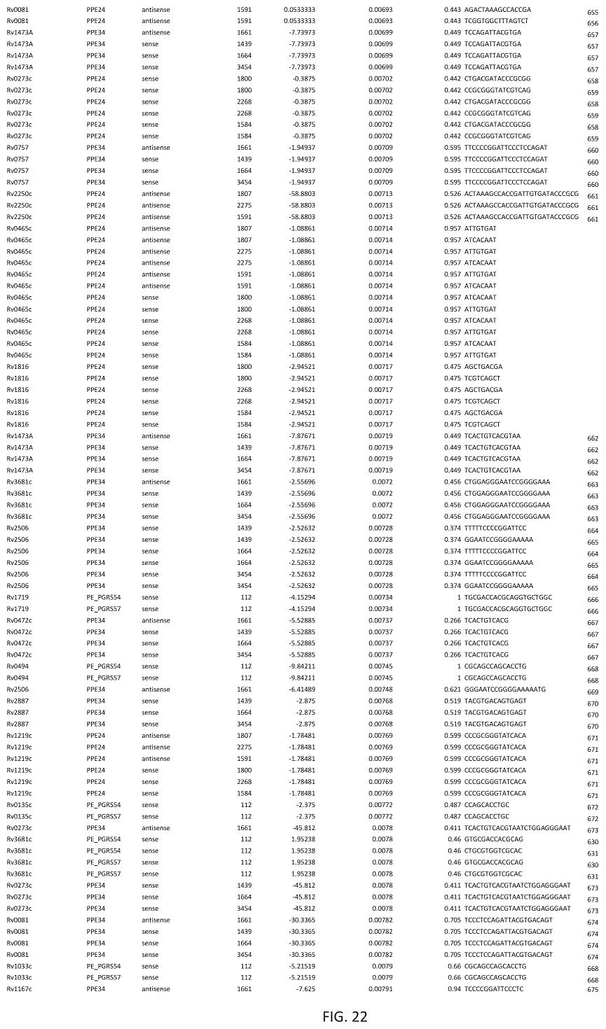

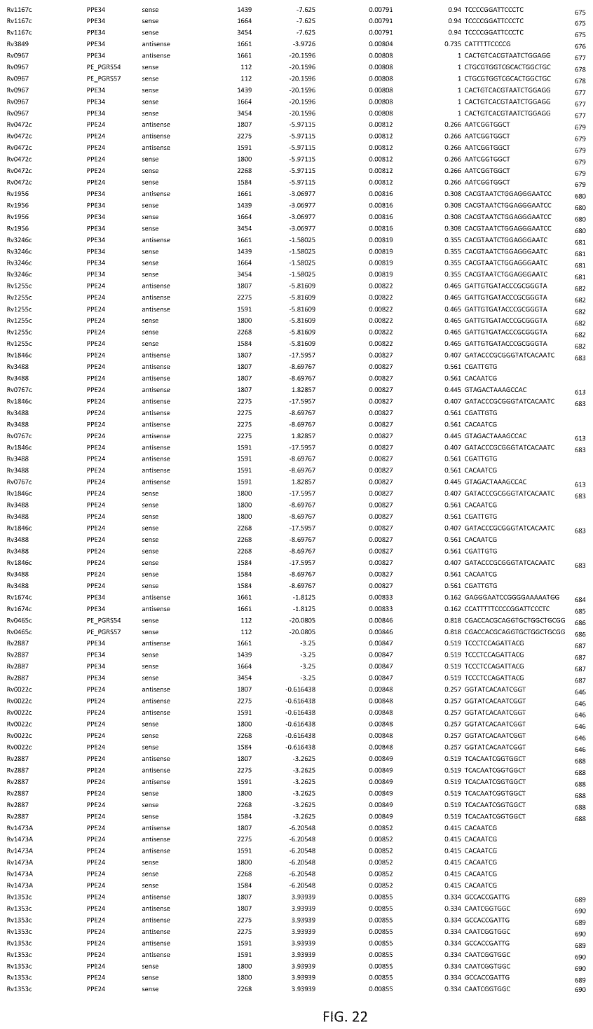

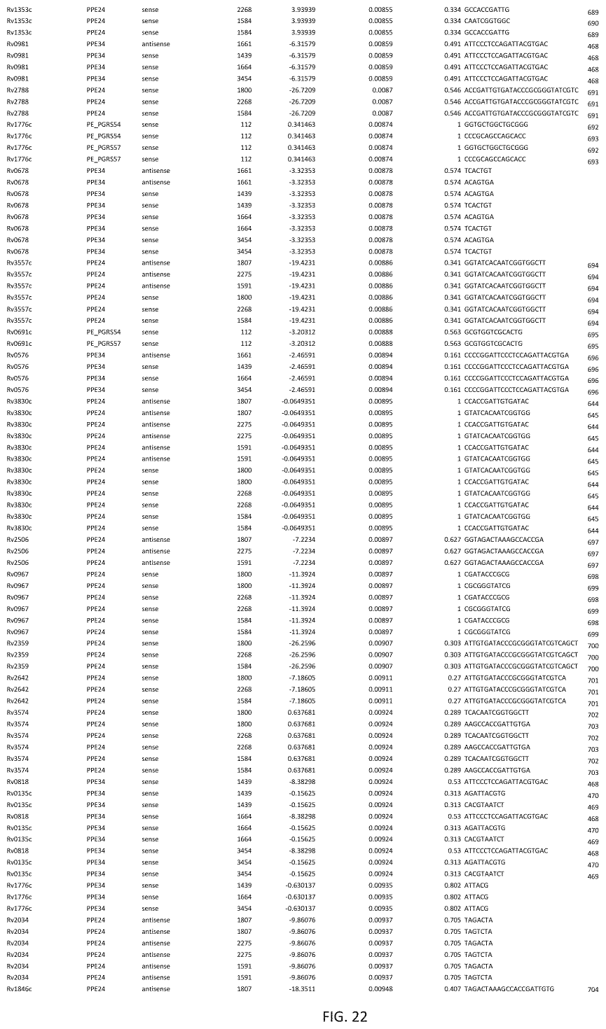

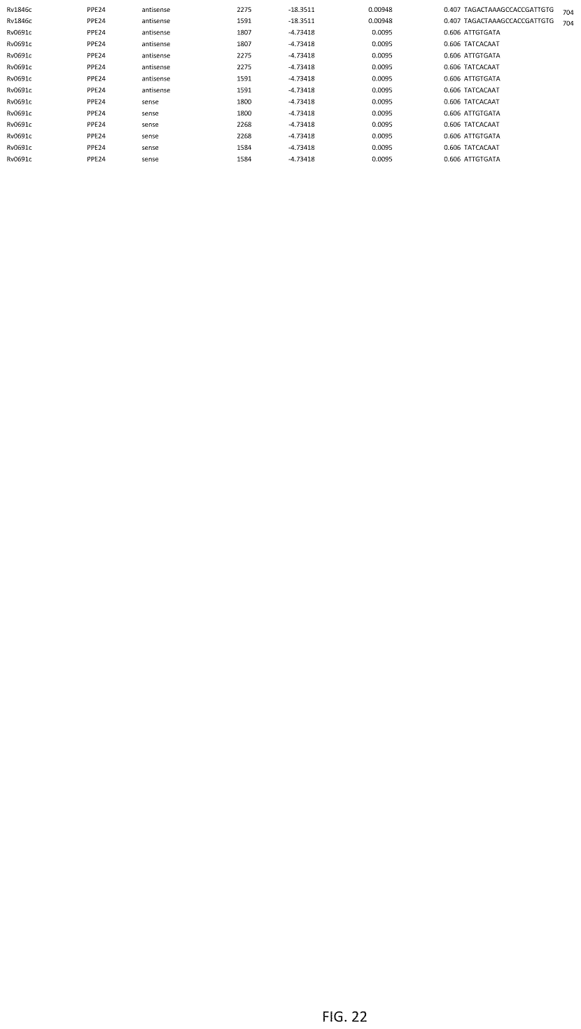

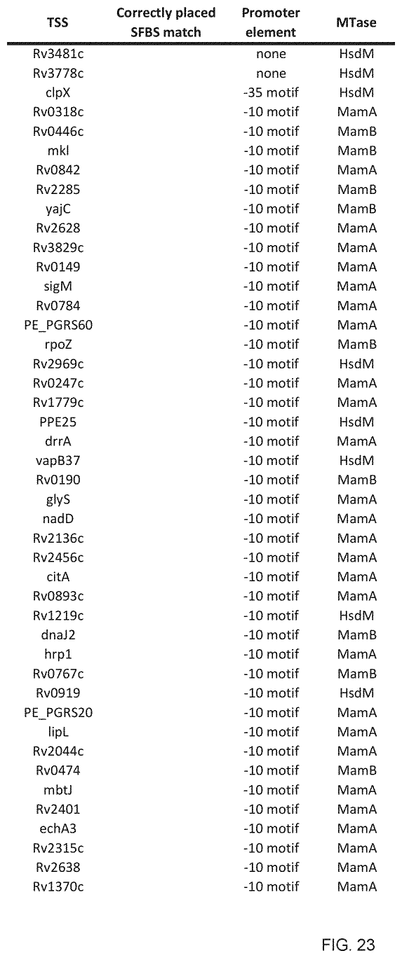

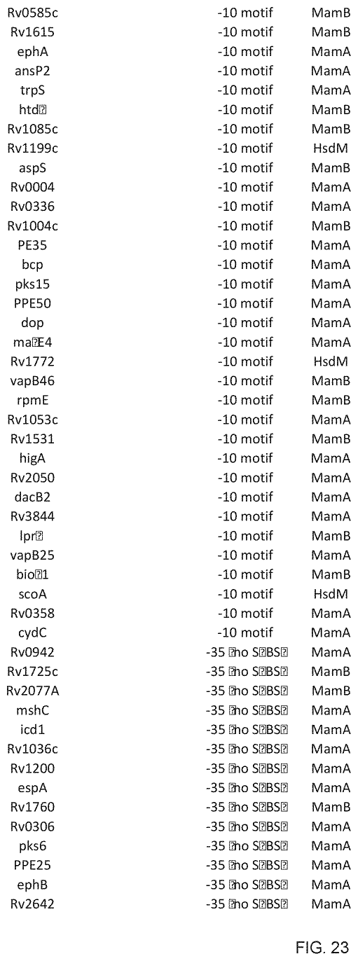

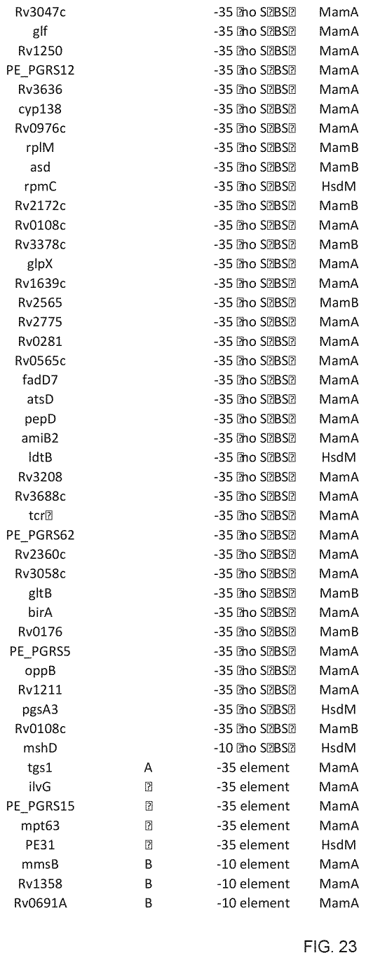

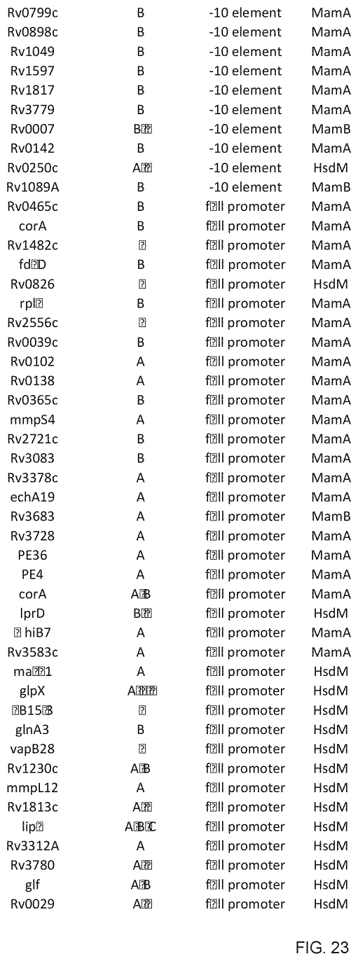

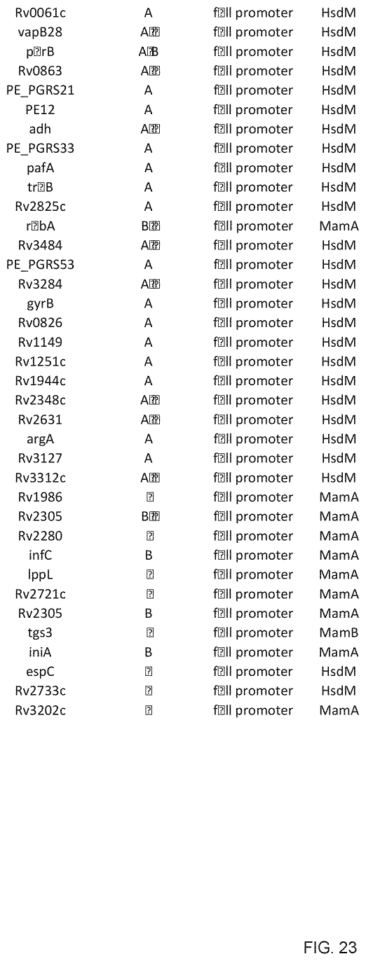

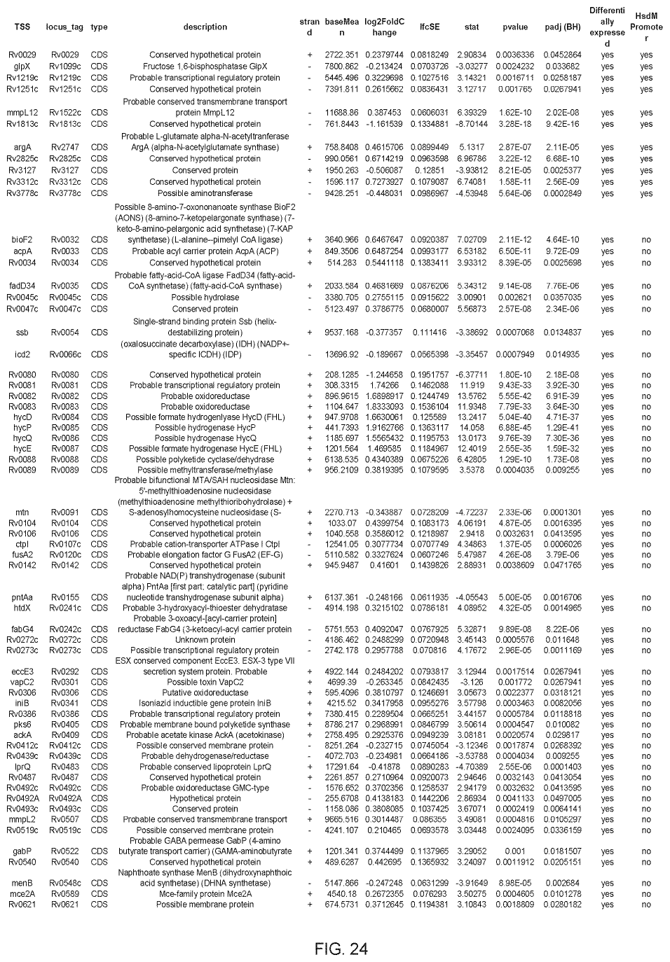

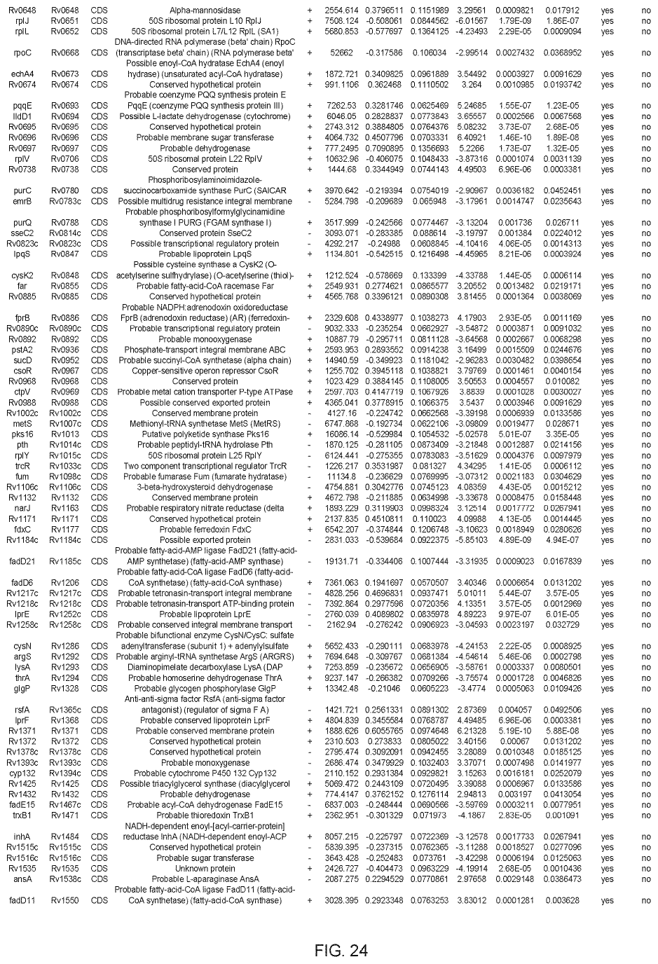

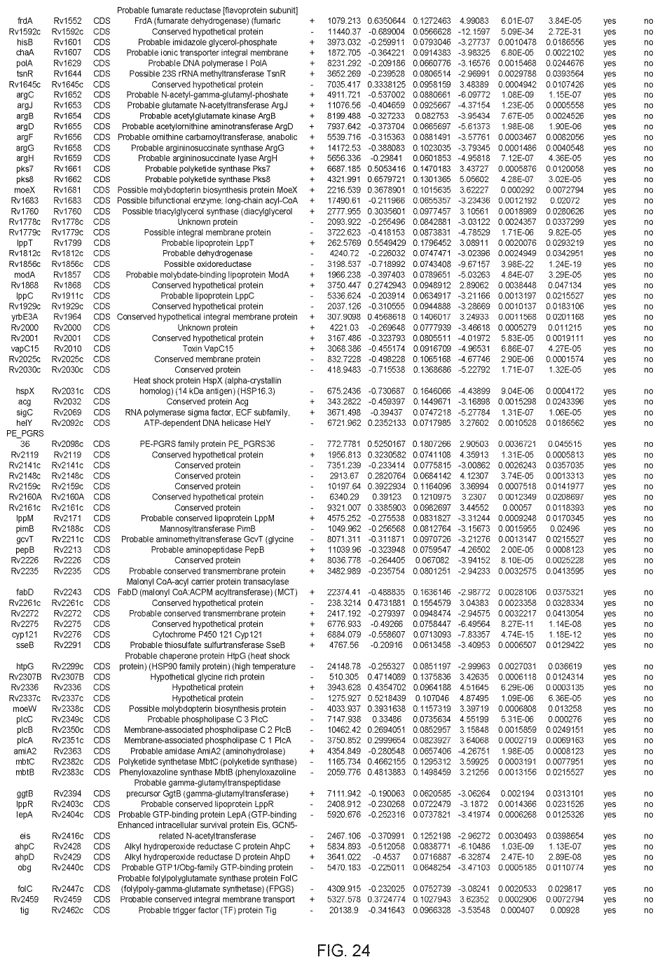

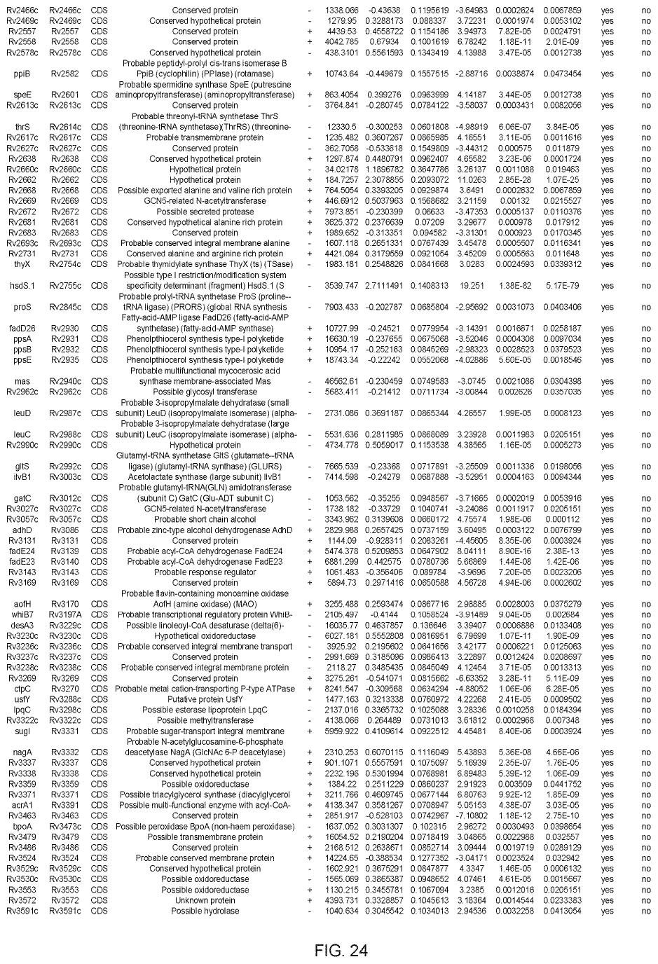

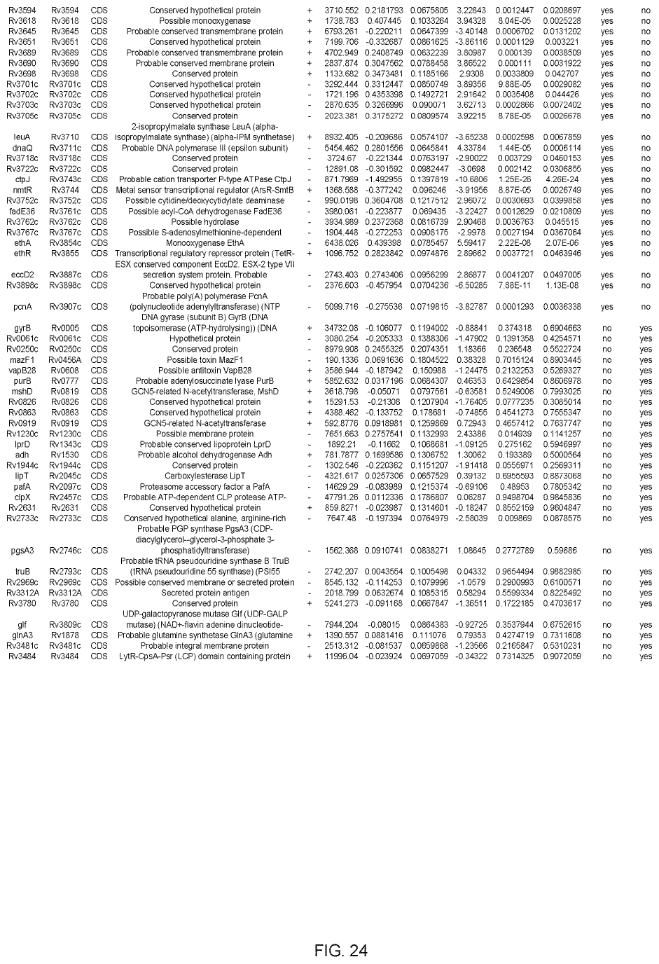

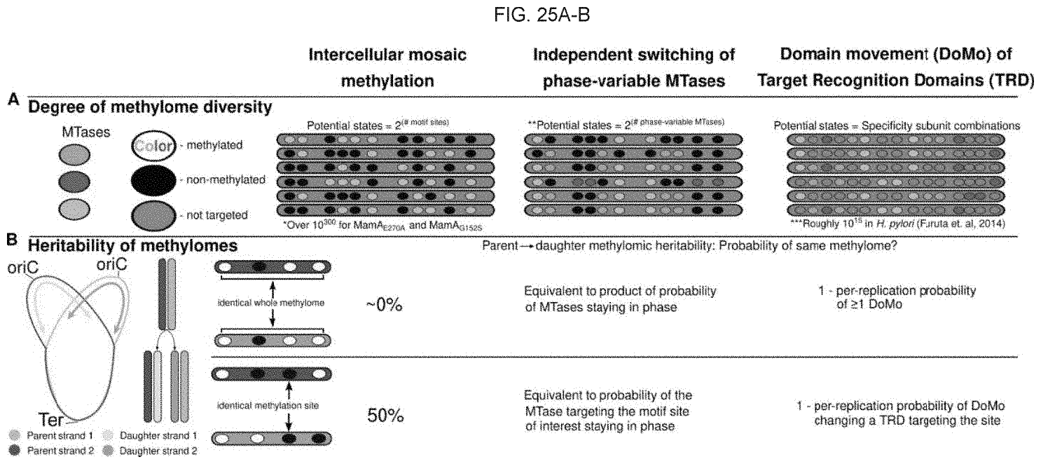

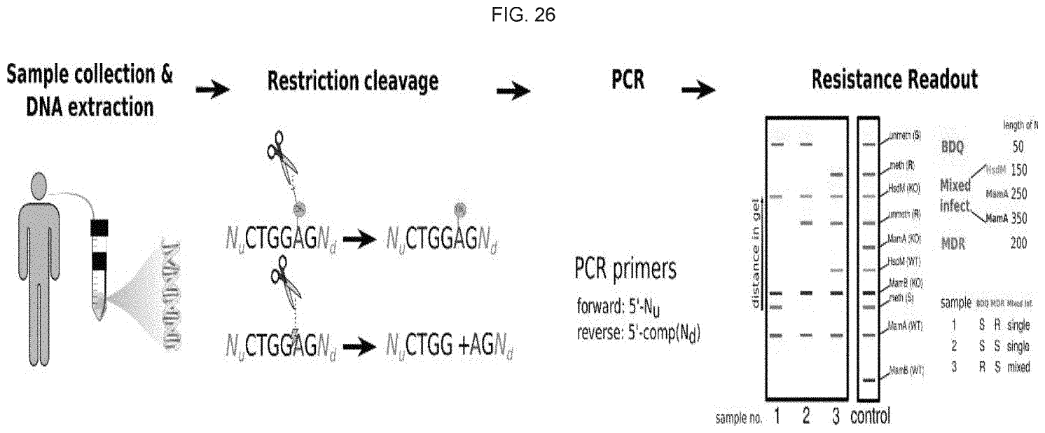

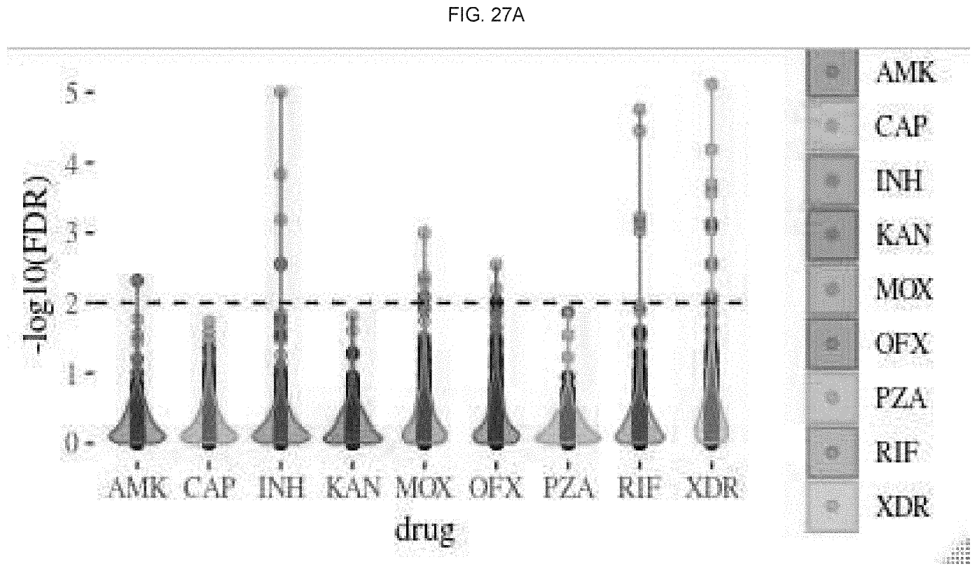

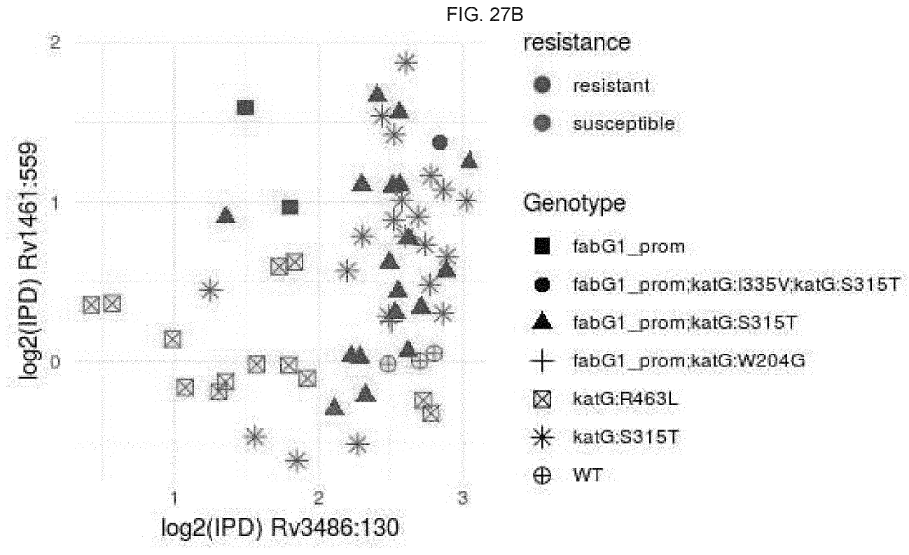

FIG. 7A-D: MTase activity patterns and genotypes across clinical and reference strains. a, Boxplot of IPD ratio distributions within MamA (top pane), HsdM (middle pane), and MamB (bottom pane) target motifs for each M. tuberculosis isolate. Boxplots are colored by mamA, hsdM, and mamB genotype. The blue line marks the mean IPD ratio of motif sites for isolates with active MTase. b, SNP-based phylogenetic trees with mutations mapped for each MTase. Isolates are colored by MTase genotype using the same colors as the boxplots in a, except for MamB, which is colored by MTase activity. The phylogeny was built using maximum likelihood on a concatenation of 22,393 SNPs with M. bovis and M. canetti as outgroups. Colors of the outer rung indicate lineage. c, Phylogeny of isolates in this study with branches colored according to the MTase activity profile. Colors of the outer rung indicate lineage. d, Density traces of sequencing kinetics for each isolate at every motif site, organized into panes by MTase (columns) and lineage (rows), and colored by the activity of their MTase.

Virulent M. tuberculosis Type Strain H37Rv Poorly Represents Methylomes of Recent Clinical Isolates.

With numerous distinct MTase activity profiles, we asked how well the commonly used virulent M. tuberculosis type strain H37Rv represents the methylomes of modern clinical isolates. In H37Rv both MamB and HsdM are inactive, while MamA is active, a rare activity profile shared with only 3% of clinical isolates (Table 3). Among the 42 isolates with a mamA knockout or knockdown mutation, a median of 3,424 MamA sites were differentially methylated from H37Rv. In contrast, the median SNP distance between H37Rv and clinical isolates was only 1,826 (FIG. 3). Thirty-five isolates had the MTase activity profile opposite of H37Rv, resulting in differential capacity for methylation from H37Rv in 5,359 to 5,484 sites, nearly triple the median SNP distance. These differentially methylated sites fall within or adjacent to over 60% of annotated genes in H37Rv. The extreme disparity between H37Rv and clinical methylomes may explain its poor transcriptional concordance with clinical isolates²⁰. Care should thus be taken when extrapolating the behavior of clinical strains from laboratory experiments on H37Rv.

TABLE 3
Methyltransferase activity by isolate count. “Isolate count” contains
the number of isolates in the dataset with methyltransferase
the activity profile specified by the values of the “MamA”,
“MamB”, and “HsdM” columns. “Active” as the value
in the “MamA”, “MamB”, and “HsdM” columns denotes
normal methyltransferaseactivity, while “Inactive”
denotes reduced or absent activity.

| Isolate Count | MamA | MamB | HsdM |
|---|---|---|---|
| 32 | Active | Active | Active |
| 13 | Active | Active | Inactive |
| 2* | Active | Inactive | Active |
| 35 | Inactive | Active | Active |
| 6** | Active | Inactive | Inactive |
| 5*** | Inactive | Active | Inactive |
| 2 | Inactive | Inactive | Active |

*Includes knockdown variant K1033T.
**Includes reference strains H37Rv and H37Ra.
***Includes knockdown variant G152S.

FIG. 8. SNP distance versus differential methylation. Dot plot comparing the number of differentially methylated sites to SNP distance between clinical isolates and virulent M. tuberculosis type strain H37Rv. The y-axis is the number of MTase motif site loci in each isolate with opposing methylation calls from H37Rv. The x-axis is the number of SNPs in each isolate, compared to H37Rv. Isolates are colored by lineage. A line with slope of 1 runs through the origin to distinguish isolates with more bases different due to SNPs (below line) from isolates with more bases different due to methylation status (above line).

### Diverse Mutations Drive DNA Methyltransferase Activity Profiles.

Cumulatively, the 93 M. tuberculosis and M. africanum isolates harbored 40 distinct mutations within the known MTase genes mamA, mamB, and hsdM/hsdS, including 32 previously unreported, see FIG. 19. Comparing the IPD ratio at each base matching the MTase target motifs across isolates revealed the effect of each variant on methyltransferase activity (FIG. 2a). The isolates had five novel knockout mutations, mamA:W136R, mamB:H770N, mamB:DELG2543, mamB:INS1181-1583, and hsdM:DEL900-909. This comparison also confirmed that variant hsdS:L119R knocks out MTase activity in the HsdM complex. Previously, hsdS:L119R had been observed only in tandem with another knockout mutation, hsdM:G173D⁴. One isolate had hsdS:L119R alone, and its HsdM motifs were unmodified (FIG. 2a, middle). Twenty-nine MTase mutations had no apparent effect on MTase activity (see FIG. 19). All isolates had at least one active MTase (FIG. 2c).

Recently, mamB D59G was reported as the sole variant in a MamB inactive isolate (SRA: ERP009820). However, we identified four isolates harboring D59G, all of which also harbored V616A. Two of the four isolates were active, and had no other mutations. The remaining two isolates were MamB inactive, and carried a 1356 bp insertion that we have identified as an IS6110 insertion sequence. One of these inactive isolates was the same isolate recently reported with mamB D59G alone. The prior study did not report the mamB insertion, likely due to their reference-mapping of short reads to call variants. However, it is unclear why their methods did not capture V616A. As MamB was active in isolates carrying mamB D59G without the insertion, we conclude the insertion was responsible for MamB knockout.

In addition to identifying knockout mutations, the comparison revealed several “knockdown” mutations, whose isolates had IPD ratio distributions consistent with neither full methylation nor unmethylation. Bases targeted by MTases with these mutations had faster kinetics than wild-type isolates yet slower than knockout isolates (FIG. 2a, 2d). Four alleles conferred knocked-down MTase activity, hsdM:K458N,E481A, mamA:G152S, mamA:E270A, and mamB:K1033T (FIG. 2a, 2d). As previously documented⁴, the variant mamA:E270A was prevalent within the East-Asian lineage, carried by 34 of 35 East-Asian isolates. Notably, the knockdown variants mamA:E270A and mamA:G152S were previously mischaracterized as knockout mutations⁴. To explain these intermediate IPD ratios, we hypothesized that MTase motif sites were heterogeneously methylated in these isolates. Reported IPD ratios are the average across multiple sequencing reads mapped to each position, which originate from different cells. Therefore, if isolate colonies contained subpopulations of cells with different methylomes, it would result in the intermediate IPD ratios we observed.

M. tuberculosis Clinical Isolates Exhibit Intercellular Mosaic Methylation

To confirm heterogeneous methylation, we ran SMALR on knockout, knockdown, and wild-type isolates. SMALR¹⁵ detects heterogeneity from SMRT sequencing kinetics by averaging the kinetics signals at multiple MTase motif sites within single sequencing reads, to calculate a “native score” for each read. The 54 mamA wild-type isolates had a normal distribution of native scores with a mean of 2.14 (FIG. 4a), indicating that most of their reads were entirely methylated.

FIG. 9. Characterizing methylation heterogeneity through SMALR. Native score (nat) is the subread-normalized natural log of IPDs. FIGS. 4a-d depict the distribution of native scores among subreads for each isolate of the specified genotype. Each colored trace represents a single isolate. FIGS. 4a-d also possess reference traces that represent theoretical distributions. FIG. 4a has a light blue trace with a mean native score identical to that of W136R, and FIG. 4b has a light violet trace with a mean native score identical to that of the wild-type. FIGS. 4c and 4d have both reference traces. Each reference trace shares the same number of measurements (isolates) and identical standard deviation as the genotype it is being compared to. Each graph also has a dotted vertical line, which marks the mean native score for all isolates of that genotype. a, Wild-type mamA. Trace peaks are overwhelmingly around 2, as expected for a fully active MTase. b, W136R, mamA-knockout genotype. Trace peaks are approximately 0. There is no second peak around nat=2, suggesting no phase variation. c, E270A genotype. A t-test revealed that the mean native score of this genotype is significantly greater than the mean native score of W136R (p<2.2e-16). A normal distribution of native scores with a mean significantly higher than that of the knockout genotype but below that of the wild-type indicates intracellular stochastic heterogeneity. d, G152S genotype. Though few isolates harbor this genotype, native scores fall clearly between 0 and 2. The mean native score is between that of W136R and wild-type, with the mean score being significantly greater than that of the former (p<2.2e-16), indicating intracellular stochastic methylation. e, Effect of methionine starvation induces heterogeneous methylation within single subreads. Heterogeneity analysis of previously published SMRT-sequencing data from a methionine auxotroph H37Rv mutant²³ (H37Rv_{ΔmetA}, blue trace). Simulated bimodal distributions scores were generated from W136R (MamA-knockout) strains and wild-type H37Rv runs (active MamA) to simulate a mixture of wholly methylated and wholly unmethylated reads (red traces). Reads were included according to the ratio of mean native IPD value in H37Rv_{ΔmetA} to mean native IPD value of wholly methylated runs (dashed vertical lines), scaled between between 0 (wholly unmethylated) and 1 (wholly methylated). f, Kinetics of Methionine-starved H37Rv_{ΔmetA} differ from phase variant simulated mixture. Peak height on the bar chart depicts the simulated bimodal distribution subtracted from the observed distribution. Dotted lines mark mean native IPD values (post-scaling) of wholly non-methylated and methylated reads. g, Stochastic versus phase-variable methylation. Conceptual illustration depicting the distinction between methylome diversity within colonies exhibiting phase-variable methylation (top) and stochastic methylation (bottom). Each gray segment represents chromosome from an individual cell within the colony. Each oval within the segment represents a motif site, illustrated as methylated (mint) or unmethylated (red).

The four isolates with knockout variant mamA:W136R each distributed normally with a mean of −0.107 (FIG. 4b) indicating that most reads were consistently unmethylated. In contrast, the four isolates with knockdown variant mamA: G152S distributed normally with a mean native score of 0.766, between mamA:W136R and mamA wild-type isolates (FIG. 4c). Reads with a native score significantly greater than that of knockout isolates yet significantly smaller than wild-type isolates have a mix of methylated and unmethylated motif sites within the read. This implies only a fraction of motif sites in genome are methylated in the cell from which the read originated. This phenomenon is called intracellular stochastic methylation¹⁵.

All isolates (n=34) with knockdown variant mamA:E270A also displayed evidence of stochastic methylation, but with a lesser methylated fraction. Their reads had a mean native score of 0.0558. This mean was significantly above that of W136R (p<2.2e-16), indicating stochastic methylation. We also analyzed heterogeneous methylated at MamB motif sites. MamB native scores in mamB wild-type and knockout isolates were similar to MamA native scores in mamA wild-type and knockout isolates, respectively (2.19, −0.192, FIG. 15A-C).

FIG. 15A-C: Methylation heterogeneity found in mamB:K1033T allele. All three graphs measure native scores for all isolates of a genotype. These isolates are represented as a multitude of colored lines, with each signifying a single isolate. There are two reference curves: a light blue curve with a mean native score identical to those of the inactive genotypes and a light violet curve with a mean native score identical to that of the wildtype. FIG. 15A: only has the light blue curve and FIG. 15C only has the light violet to avoid redundancy. FIG. 15B has both curves. Each reference curve shares the same number of measurements (isolates) and identical standard deviation as the genotype it is being compared to. FIG. 15A, The top left graph displays the wildtype. The mean native score is 2.19, similar to that of the mamA wildtype isolates. There are no signs of stochastic heterogeneity or phase variation. FIG. 15B, The top right graph represents the K1033T genotype with only one isolate. This graph possesses a dotted vertical line identifying the mean native score and a solid black line that identifies the mean native score of the inactive genotypes. It has low MamB activity, and displays a similar native score to that of the E270A genotype of mamA, which also has low MTase activity. K1033T has a mean native score of 0.299, significantly greater than the mean native score of knockout genotypes (p<2.2e-16) and has a lower score than that of the wildtype, suggesting stochastic heterogeneity. FIG. 15C, The bottom graph is a representation of all mamB-knockout genotypes. Despite these isolates having different nonsynonymous mutations, they are cleanly overlaid on top of each other. Their mean native score is −0.192, which is what we expect of an MTase knockout genotype. We see no phase variation or stochastic heterogeneity.

MamB possessed one knockdown genotype, mamB:K1033T, found in a single isolate. Like mamA:E270A, mamB:K1033T had a significantly greater native score than that of the knockout genotypes (p<2.2e-16). HsdM motif sites occurred too infrequently across the genome for same-read analysis of multiple sites with SMALR preventing us from drawing conclusions about heterogeneity at HsdM motif sites.

Because the methylation of each MTase motif site is independent between cells, intracellular stochastic methylation results in diverse combinations of methylated motif sites within an isogenic colony. We call this epigenetic mosaicism “intercellular mosaic methylation”. Intercellular mosaic methylation is notably distinct from phase variant MTase knockout, in which a subpopulation of cells have an inactive MTase. Phase variant MTase knockout causes a portion of an isolate's reads to be entirely methylated, and another portion to be entirely unmethylated. Native scores of phase variant MTase knockouts would distribute bimodally (FIG. 4g). While phase variant MTase knockout has been observed in many bacteria, intracellular stochastic methylation has previously only been observed in a single species, Chromohalobacter salexigens¹⁵.

Next, we asked whether nutrient restriction could cause intercellular mosaic methylation in isolates with wild-type MTase function. We ran SMALR on published kinetic data from metA-knockout H37Rv methionine auxotrophs (ΔmetA) SMRT-sequenced following 5 days of methionine starvation²¹.

To test for intercellular mosaic methylation in ΔmetA we compared its native score distribution to a mixture of wholly methylated and wholly unmethylated reads (FIG. 4e). This simulated mixture was sampled from mamA wild-type and knockout isolates, in proportion to produce the same mean. The ΔmetA native score distribution was distinct from the simulated mixture, with fewer fully methylated reads and more partially methylated reads (FIG. 4e,f). These results are consistent with a mixture of fully methylated reads inherited from bacilli born prior to methionine deprivation and stochastically methylated reads from daughter strands that underwent re-methylation following starvation. These results suggest that intercellular mosaic methylation can be induced by nutrient limitation (at least for methionine, and presumably others nutrient constraints that limit flux through the adenine methyltransferase reaction), providing a mechanism of environmentally-induced phenotypic heterogeneity.

### Anomalous Methylation Patterns in Orphan MTase Motif Sites

Next, we surveyed all common motif sites (present in greater than or equal to 75 isolates, n=4,486; FIG. 21) to observe how they varied across isolates and find common attributes among sets of sites following in vitro culture. Across isolates of the same genotype, we asked whether IPD ratio distributions were similar or varied. IPD Ratio distributions were consistent within sets of isolates with the same genotype, except for knockdown mutants (FIG. 5A upper image), highlighting these mutants as pertinent targets for further inquiry. Most of the interesting isolates harbored knockdown mutations in mamA, although a few wild-type hsdM isolates exhibited methylation patterns inconsistent with the rest (FIG. 5A middle image).

Assessing the consistency and magnitude of IPD ratios within active wild-type isolates for each MTase across motif sites revealed three interesting features. First, while most motif sites are primarily methylated (light, yellow), a subset had significantly lower median IPD (FIG. 5A upper and middle images). Second, among the motif sites that were mostly methylated, some had lower IPDs in a subset of isolates (FIG. 17A).

FIG. 17A-C: Analysis of hypomethylated and hypervariable motif sites: FIG. 17a, Distribution of normalized IPD ratios at consistently hypomethylated MTase motif sites, across isolates. Only loci present in at least half of the clinical isolates were included. Histograms of the kinetics signal of isolates at specific genome sites matching the target motifs of known methyltransferases (MTases). The IPD ratios are normalized to the mean IPD ratio of all adenines within each isolate, and log 2 transforming the data. The bin width is 0.2 and the bars are colored by the Bayesian classification of each locus within each isolate (methylated, hypomethylated, and indeterminate). In each histogram only isolated that possessed the locus and carried an active genotype of the relevant MTase were included. FIG. 17b, Distribution of standard deviation size of kinetics (log 2 of the IPD Ratio) for each common (n>50) motif sites across isolates with the relevant MTase active. MamB is least frequently hypomethylated and the three distributions appear similar outside of outliers, so it was used to determine the standard deviation (of standard deviation size). Any sites with a standard deviation size >3 standard deviations above the (MamB) mean. These were considered as variable epigenetic loci, and candidates for epigenetically-driven phenotypic differences (FIG. 5 and FIG. 7). FIG. 17c, Point plot of each common motif site position according to its mean and standard deviation across isolates with active MTases. Points are colors according to whether they are hypomethylated (blue), hypervariable (red), hypervariable and hypomethylated (purple), or meet none of these criteria (grey). The top 5 most variable motif sites and bottom 5 mean sites for each MTase are labelled, if they classified as hypervariable and/or hypomethylated.

The converse was also true. Some motif sites with low median IPDs had higher IPDs in a subset of isolates. Third, knockdown mutations had distinct methylation profiles with IPDs higher than knockout mutants and lower than wild-type. All three features were more pronounced in motif sites of orphan MTases than of MamB (FIG. 5).

We then sought to identify motif sites that were hypervariable across strains with active MTases. We reasoned that such hypervariability would indicate differential selection for methylation status, highlighting interesting differences between strains. To find hypervariable sites, we calculated the standard deviation in IPD ratio across isolates (variation) for each shared MTase site. Variation across isolates for each qualifying motif site was compared against the distribution of variation in MamB sites (FIG. 17B), because they distributed normally, with few outliers. Out of the 4,486 shared MTase motif sites, 351 had variation at least three standard deviations above the mean variation among MamB sites, see the data listed in FIG. 20. These hypervariable sites fell within 204 coding regions, and within potential promoters upstream (within 100 bp) of 42 TSSs, see FIG. 20. Only seven hypervariable sites were MamB motifs. This specificity of hypervariable motif sites to MamA and HsdM is consistent with the view of orphan MTases as epigenetic mediators of important physiological processes²².

FIG. 20 legend:

| | |
|---|---|
| RefLoc | References the distance to the nearest |
| i | CDS boundary |
| TSSid | The CDS downstream of the TSS ahead of |
| | which the locus falls, and the number of |
| | base pairs upstream of the TSS |
| | that the targeted adenine is positioned. |
| TSS | The CDS downstream of the TSS within which the |
| | locus falls |
| median | The median log2(IPD Ratio) at the |
| | specified locus among all sites with |
| | the specified Mtase active |
| mean | The mean log2(IPD Ratio) at the specified locus among |
| | all sites with the specified Mtase active |
| sd | The standard deviation of log2(IPD Ratio) |
| | at the specified locus among |
| | all sites with the specified Mtase active |

FIG. 20: All fields referencing log 2(IPD Ratio) were calculated using the run-specific scaled IPD Ratio. Every isolate was scaled such that the mean IPD Ratio of all non-motif adenines==1.

We then contrasted variability of methylation at each motif site across isolates of the same activity level (knockdown, knockout, or wild-type, FIG. 5d). For knockdowns, we only considered mamA:E270A mutants, since no other knockdown mutation occurred frequently. MamA motif sites varied more across mamA:E270A isolates than across mamA wild-type or knockout isolates.

FIG. 10. DNA methylation patterns at orphan MTase motif sites discovered through comparative methylomics. Heat maps of sequencing kinetics for a, MamA, b, HsdM, and c, MamB motifs. y-axis is all common motif sites, descending according to median sequencing kinetics (log 2 IPD ratio). Isolates (x-axis) are sorted from left to right by activity level, lineage, and genotype in decreasing priority. Lineages are Indo-Oceanic (IO), East-Asian (EAS), East-African-Indian (EAI), Euro-American (EUR), Ethiopian Lineage 7, and the M. africanum lineages 5 and 6. Dots on the rotated plot adjacent to the heatmap express the median log 2(IPD Ratio) for each site across isolates. Darker and lower dots indicate a lower median log 2(IPD Ratio). Red arrows mark isolates with wild-type or near wild-type MTase activity, yet exhibit hypomethylation at more motif sites (dark bands) than other wild-type isolates. Blue arrows mark two isolates with significantly fewer hypomethylated motif sites than other isolates with wild-type HsdM activity, for unknown reasons. The green arrow in the MamB plot marks an isolate with an IPD ratio significantly higher than expected for a knockout isolate. d, Distribution of standard deviation (SD) sizes among MamA motif sites across isolates with one of three methylation activity levels: MTase knockdown from the E270A mutation common to East-Asian isolates, the W136R Knockout mutation, or one of the genotypes encoding MamA with wild-type methylation activity. e, Position (x-axis) in a representative genome and variability (SD of log 2(IPD Ratio)) in sequencing kinetics across isolates with active MTase (y-axis) at common motif sites (present in 75 or more isolates). Motif sites within three SD of the mean for MamB motifs are grey, and the outliers (>3 SD from mean) are highlighted in red. CDSs within which each of the top 10 most variable sites for each MTase occur are labelled, along with their palindromic partner motif site.

FIG. 18: Sigma Factor Binding Site Motif and MTase Motif overlap. Overlap of MTase and SFBS motifs for M. tuberculosis Sigma factors. Histogram height corresponds to the number of TSSs harboring an overlap at that position. Only those appearing in at least 40 isolates are depicted. Bar color represents whether the SFBS motif was for a −35 or −10 element. The −10 and −35 regions are highlighted with dashed vertical lines in each plot.

### Hypomethylated MTase Motif Sites are Rare Yet Remarkably Consistent Across Isolates

Every isolate possessed a handful of unmethylated motif sites targeted by otherwise active MTases. Hypomethylated sites like these have previously been found in M. tuberculosis³ and many other bacteria^{9,22}. Per active isolate there were on average 20.7 hypomethylated HsdM sites, 13.4 hypomethylated MamA sites, and 0.289 hypomethylated MamB sites (FIG. 16A-D), comparable to previous reports³. However, while rare, these hypomethylated motif sites showed remarkable consistency, with the same motif sites hypomethylated in multiple isolates. The most conserved hypomethylated site was in mmpL4, 1718 bp from its start. Despite having a MamA target motif, this site was unmethylated in 51 MamA active isolates (Table 1, see FIG. 8). In total, 34 MamA sites and 57 HsdM sites were consistently hypomethylated (cumulative binomial p-value <4.19E-07, Table Y:

| | | | | Nearby |
|---|---|---|---|---|
| Position | Hypomethylated | All | P-value | Motif |
| 1719 | 51 | 51 | 2.18E−126 | No |
| 1716 | 49 | 51 | 2.3367E−118 | No |
| 7 | 39 | 51 | 1.22701E−85 | No |
| 472 | 38 | 50 | 2.73095E−83 | Yes |
| 475 | 35 | 50 | 1.23593E−74 | Yes |
| 1272 | 31 | 35 | 2.14038E−72 | No |
| 1275 | 26 | 35 | 5.92888E−57 | No |
| 447 | 20 | 33 | 2.88804E−41 | No |
| 1664 | 13 | 20 | 7.05078E−28 | No |
| 23 | 14 | 31 | 8.0244E−27 | No |
| 598 | 13 | 25 | 4.65494E−26 | Yes |
| 325 | 12 | 18 | 4.9305E−26 | No |
| 1069 | 13 | 43 | 3.09114E−22 | Yes |
| 2728 | 13 | 50 | 2.93266E−21 | No |
| 796 | 11 | 49 | 2.03632E−17 | No |
| 880 | 8 | 13 | 2.46189E−17 | No |
| 416 | 10 | 50 | 2.07856E−15 | Yes |
| 1469 | 9 | 44 | 4.24545E−14 | No |
| 4 | 9 | 51 | 1.78287E−13 | No |
| 1661 | 7 | 20 | 4.21414E−13 | No |
| 1662 | 7 | 24 | 1.85898E−12 | No |
| 1066 | 8 | 43 | 2.53081E−12 | Yes |
| 642 | 8 | 48 | 6.486E−12 | No |
| 389 | 8 | 50 | 9.17186E−12 | Yes |
| 787 | 8 | 51 | 1.08451E−11 | No |
| 3454 | 4 | 4 | 1.39369E−10 | No |
| 1309 | 7 | 46 | 2.69087E−10 | No |
| 595 | 6 | 25 | 2.75514E−10 | Yes |
| 877 | 5 | 13 | 6.02318E−10 | No |
| 1439 | 4 | 6 | 2.07906E−09 | No |
| 10 | 6 | 48 | 1.78409E−08 | No |
| 712 | 6 | 51 | 2.59529E−08 | No |
| 1409 | 3 | 3 | 4.05624E−08 | No |
| 37 | 4 | 14 | 1.35722E−07 | No |

Table Y summaries a hypomethylation analysis. Consistently hypomethylated MTase motif sites across 93 clinical Mycobacterium tuberculosis and Mycobacterium africanum clinical isolates. MTase motif site loci were assigned by our methylome annotation pipeline, using proximal H37Rv gene references transferred by Rapid Annotation Transfer Tool (http://ratt.sourceforge.net/). Consistently hypomethylated loci were classified as unmodified by our Bayesian analysis in a significant number of isolates in which the relevant MTase was mostly active. Significance was calculated using the cumulative binomial test, setting the number of MTase-active isolates where a locus was present as the number of trials, and the number of said isolates where the locus was hypomethylated as the number of successes. At 0.01 significance level, the threshold for p-value for significance was 4.72E-07, after a Bonferroni correction for the number of loci tested. Sheet one contains hypomethylated MamA motif site loci. Sheet two contains the hypomethylated MamB loci, and sheets three contains hypomethylated HsdM loci.

This consistency would be unlikely if hypomethylation occurred randomly, suggesting there are conserved mechanisms blocking methylation at these sites.

FIG. 16A-E: Evaluation of Bayesian Classifier. FIG. 16a, MamA, FIG. 16b, HsdM, and FIG. 16c, MamB. Methyltransferase target bases in each of 93 Single Molecule Real Time (SMRT) sequenced clinical isolates were classified as Methylated (not shown), Hypomethylated, or Indeterminate based on their sequencing kinetics. Stacked histograms depict the proportion of motif sites (y-axis) classified as Hypomethylated (red) or Indeterminate (purple) in each isolate (Each bar represents an isolate, x-axis). Isolates are sorted by the proportion hypomethylated motifs. Only isolates with an active genotype for each MTase are included in the histograms. FIG. 16d, This histogram reports the distribution of IPD ratios among bases within the target motifs of known methyltransferases HsdM, MamA, and MamB, after normalizing the IPD ratios of each base to the mean IPD ratio of all adenines within the isolate, and log transforming the data. The bin width is 0.1 and the bars are labeled by the Bayesian classification of each base. The isolate shown is a clinical strain of M. tuberculosis with an active genotype of all three Methyltransferases. FIG. 16e, Violin plot showing the distribution of coverage at MTase motif sites, aggregated from all clinical isolates. Only MTase sites whose respective MTase had an active genotype in their respective isolate were included. MTase sites that were classified as Indeterminate by the Bayesian classifier were grouped separately (purple) from the remaining sites (grey). The MTase sites with a definitive classification call had a higher mean coverage (68.2) than the MTase sites called Indeterminate (54.7), significant at 0.01 confidence level (Welch's two sample t-test p-value <2.2e-16, one sided).

### Transcription Factor Occlusion Explains Most Hypomethylated Sites

In other bacteria, hypomethylation results from transcription factor occlusion blocking the MTase when their respective target motifs match the same site in the genome⁸⁻¹⁰. To determine if this was the case in M. tuberculosis, we scanned the context sequence of each consistently hypomethylated site for transcription factor binding sites (TFBSs) motifs previously characterized in M. tuberculosis¹⁹. All 58 consistently hypomethylated HsdM loci matched at least one significant TFBS motif (p-value <0.0001, converted log-likelihood ratio score), while only 14 of the 34 consistently hypomethylated MamA loci significantly matched a TFBS motif (p-value <0.0001, converted log-likelihood ratio score; Table 1, see FIG. 8). The other MamA loci did match TFBS at lower significance levels (FIG. 22).

FIG. 22 shows data for Transcription factor overlap: Significant transcription factor binding motifs scanned with FIMO (Find Individual Motif Occurrences) (http://meme-suite.org/doc/fimo.html) in the context sequences of consistently hypomethylated MTase motif sites. MTase motif site loci were assigned by our methylome annotation pipeline, using proximal H37Rv gene references transferred by RATT (http://ratt.sourceforge.net/). Consistently hypomethylated loci were classified as unmodified by our Bayesian analysis in a significant number of isolates in which the relevant MTase is mostly active. Context sequences for each locus were defined as the 20 nucleotides flanking each side of the base targeted for methylation in each MTase motif site. In cases where the context sequence for a locus varied between isolates, the most common context at that locus was used. Transcription Factor Binding Motifs (TFBSs) were characterized by Minch et al. through ChIP-seq experiments on laboratory strain H37Rv, and MEME (http://meme-suite.org/tools/meme). Minch et al. (https://www.nature.com/articles/ncomms6829) characterized both palindromic and non-palindromic motifs for each transcription factor. Both versions of these motifs were scanned for matches. Only TFBS motifs with an E-value below 0.01 were scanned for matches. All matches with a p-value (converted log-likelihood ratio score) below 0.01 are reported, though only matches with a p-value below 0.0001 were included in Table 1.

The abundance of TFBS matches at HsdM motif loci may be due to the lower stringency of its motif (HsdM: GATNNNNRTAC (SEQ ID NO:21), MamA: (SEQ ID NO:6) CTGGAG). Notably, the transcription factor binding site (TFBS) motif of oxidation-sensing regulator mosR (Rv1049)²⁸ matched multiple hypomethylated MamA and HsdM loci, and the mosR gene itself had a hypomethylated MamA locus 7 bp upstream of its TSS (Table 1).

One particularly intriguing example of site-specific hypomethylation was cobK:304, the HsdM motif site 304 bp inside the gene cobK. This locus was hypomethylated in 50 HsdM active isolates, yet methylated in 18 isolates (Table 1, see also FIG. 8). The IPD ratio at cobK:304 across HsdM active isolates was bimodal (FIG. 6a), supporting this finding. The 18 cobK:304 methylated isolates were all Indo-Oceanic, and grouped together in our phylogenetic tree (FIG. 6b). The context sequence around cobK:304 matched the binding site motif of transcription factor mntR (Rv2788) (q-value=0.0053, Table 1). This explained the 49 cobK:304 hypomethylated isolates, as mntR bound to that site and prevented HsdM from methylating it. However, this still did not explain why cobK:304 was methylated in the Indo-Oceanic isolates. Genotyping mntR revealed that all 18 Indo-Oceanic isolates shared the variant mntR:Q131STOP (FIG. 6b), a nonsense mutation found previously in Indo-Oceanic isolates¹¹ that truncated the mntR by introducing an early stop codon.

On the other hand, in some bacteria hypomethylation has also been observed when MTase motif sites were in close proximity¹². To find such instances, we scanned consistently hypomethylated loci for nearby MTase motifs (Table 1, see also FIG. 8), including loci cobK:304. In most isolates, cobK:304 was only 8 bp distant from another HsdM site, cobK:312 (together making four palindromic motif matches within cobK). In these isolates, cobK:312 was methylated while cobK:304 was hypomethylated. However, in Indo-Oceanic isolates the HsdM motif at cobK:312 was destroyed by a nearby deletion. If MTase crowding was responsible for the hypomethylation of cobK:304, then the removal of cobK:312 in Indo-Oceanic isolates may be responsible for cobK:304 methylation in that lineage. As cobK:304 was consistent with both previously described phenomena, it is uncertain whether its hypomethylation was caused by its neighboring MTase motif, or by occlusion by transcription factor sirR.

FIG. 11. Evidence of Transcription Factor occlusion at hypomethylated MTase sites. a, Histogram showing the distribution of IPD ratios at the HsdM motif locus cobK:304, 304 bp downstream from the start codon of gene cobK. Included isolates have active HsdM and possess the HsdM target motif at the cobK:304 locus. IPD ratios are normalized to the mean IPD ratio of adenine bases in their respective isolates (excluding bases targeted by known MTase motifs), and transformed by log base 2. The histogram uses a bin width of 0.1. Red bars count isolates classified as “hypomethylated” at the cobK site, while green bars count isolates classified as methylated at the site. b, Phylogenetic tree of the 90 clinical and reference M. tuberculosis isolates and 3 M. africanum isolates included in this study, along with outgroups M. bovis and M. canetti. Isolates are colored in the middle ring by their methylation status at the HsdM motif site cobK:304. Red isolates are classified as hypomethylated at the cobK site; green isolates are classified as methylated at the site, and grey isolates either have an inactive HsdM methyltransferase, or are missing the HsdM target motif 304 bp within their cobK gene. Isolates are colored in the outer ring by the genotype of their mntR (Rv2788) gene. mntR encodes for a transcription factor whose binding motif matches the context sequence of the cobK 304 site (p-value 2.63E-05, converted log-likelihood ratio score). Gold isolates had the variant mntR Q131STOP, a nonsense mutation that introduces an early stop codon that truncated the gene and presumably knocked out its function. The blue isolates do not have a nonsense mutation, though one isolate had the missense mutation mntR P149L.

Table 1, Illustrated as FIG. 8, Consistently Hypomethylated MTase Motif Sites Across Clinical M. tuberculosis Isolates.

The top 20 most significant hypomethylated loci from each MTase. For each methyltransferase (“MTase”) motif target locus (“Gene”, “Sense”, and “Position”), we counted the number of isolates in which the isolate was hypomethylated and the total number of isolates that possessed the locus (“Hypomethylated”). This fraction was used to perform a cumulative binomial probability test (“P-value”). Loci with p-values below 4.72E-07 were considered significant at 0.01 significance level, after Bonferroni correction for multiple hypothesis testing. Loci were assigned by our methylome annotation pipeline using H37Rv reference annotations transferred from RATT³⁰. For each palindromic pair, the locus with the most significant hypomethylated fraction is reported. In case of a tie, the locus on the same strand as the gene is reported. The fraction of active isolates hypomethylated at the partner site is included (“Palindrome”). The surrounding 20 bases of each loci were scanned for transcription factor binding site motifs previously characterized in M. tuberculosis²⁷. The most significant motif match was included (“Top TF”). Only transcription factor binding motifs with an E-value below 0.01 were scanned for, and only matches with a p-value (converted log-likelihood ratio score) below 0.0001 were reported. MTase motif loci less than 100 bp from another locus targeted by the same MTase were labeled (“Yes” in column “Nearby Motif”). Genes that were previously reported⁴ to contain frequently hypomethylated sites are marked with an asterisk.

### Methylation is Widespread and Distinctly Patterned at Promoters

Next, we systematically probed promoters with MTase to identify common configurations between motif sites and characterized TSSs^{31,32}. Within promoter regions (<50 bp upstream from the TSS). Targeted adenines of MamA and HsdM motifs had distinct peaks at the edges of the −10 element (FIG. 7a). The MamA peak comprised Twenty-two promoters coincident with the −10 element in the configuration that has been shown previously to modulate transcription (4-5 and 7-8 bp upstream from TSS, (FIG. 7a, FIG. 11a). These included the four shown to affect transcription (FIG. 11a, blue stars). Notably, none of these four were hypomethylated or hypervariable, indicating that lack of anomalous methylation in vitro does not preclude a role in transcriptional regulation. Common (n≥75) HsdM motif sites overlapped with the −10 element of 32 promoters. While nineteen of these match those recently reported¹⁴, 13 are novel. These HsdM motif sites frequently overlap with the −10 promoter element in a configuration analogous to that common in MamA motifs, but on the distal (−10 to −13 bp) end (FIG. 7a). In total, 353 genes have common (in ≥75 isolates) promoter MTase motif sites (FIG. 23).

Next, we scanned for SFBS motifs overlapping promoter MTase motif sites. Sigma factors SigA and SigB overlapped MTase motif sites most frequently (FIG. 7b), though SigC, SigD, SigI, and SigF overlapped motifs sites as well (FIG. 22). MamB motif sites rarely overlapped SFBSs, while orphan MTase motif sites frequently did (FIG. 7a-c). MamA motif sites were more frequent in promoter regions than HsdM sites, however HsdM sites more frequently overlapped a SFBSs (FIG. 7c, perhaps explainable by both HsdM and SigA target motifs including the dinucleotide “TA”). These findings suggest a potential mechanism for cis regulation of dozens of genes with orphan MTase motifs in M. tuberculosis.

FIG. 12A-E. Configuration of orphan MTase motif sites at promoters suggest widespread epigenetic influence on transcription: FIG. 13A, Consistent MTase-SFBS-promoter configuration. For each MTase, a frequency plot is displayed for occurrences of unique MTase motifs at distances upstream of the TSS. The canonical SigA binding motif is superimposed for conceptual clarity, but other SFBSs, and loci with no known SFBS in the −7 to −12 bp window upstream of annotated TSS are also included. Count reflects total number of methylated motif for each MTase at each position across all isolates (only counted once per promoter) and TSSs. The −10 region where sigma factors typically bind is highlighted with the sigma factor A binding motif indicated. Motifs for each of the two MTase motifs overrepresented (peaks) are shown in the orientation and positions that explain the observed peak patterns. FIG. 14B, Histogram of the number of promoters with the −10 element overlapping a MTase motif site in at least 30 isolates, for each MTase and sigma factor. FIG. 15C, variability (SD of log 2(IPD Ratio) across isolates) in sequencing kinetics across isolates with active MTase (y-axis) for common (≥75 isolates) promoter motifs positioned according to their distance upstream of their TSS (x-axis). Motif sites within three SD of the mean for MamB motifs are grey, and the outliers (>3 SD from mean) are highlighted in red, and labelled with downstream gene. FIG. 16D, Stacked histograms of number of genes harboring promoter motif sites for each MTase. Darker shades indicate progressively substantiated promoters. In “full promoters” MTase motifs overlapped with a SFBS that is part of a classical promoter architecture (Methods). Element matches overlap either the −10 or −35 SFBS (but not both) and in the correct position. Location matches are in position to overlap with −10 or −35 elements but do not coincide with known SFBS motifs. Motif matches coincide with SFBSs but not in the expected position with respect to TSS. FIG. 17E, Differentially expressed genes in recent ΔHsdM study. All HsdM promoter motifs are positioned according to position within the promoter and Benjamini-Hochberg adjusted −log 10(p-value). Motif sites in promoters of significantly differentially regulated (p<0.05) genes are colored red, and their motif sites overlapping with the −10 element (7 to 13 bp upstream of TSS) are labelled. The two genes without overlapping sites the −10 element have both their motif sites labelled (if within 50 bp).

FIG. 11: Methylomic variation at promoters harboring orphan MTase motifs. Heatmaps depicting degree of methylation (scaled log 2 of IPD ratio averaged across reads) across all 93 clinical isolates (columns) at all common (present in ≥75 isolates) promoter (≤50 bp upstream of a TSS) motif sites (rows). Hypervariable motif sites (>3 s.d. above MamB motif site mean variability, FIG. 5e) are marked by red font. The coloring scale of the heatmap boxes max out at the median scaled IPD across all motif sites across all isolates with active MTase (calculated separately per MTase), and bottom out at 0 (corresponding to no methylation). Activity of isolates are indicated at the bottom of each heatmap. Isolates within each heatmap are sorted first by activity, and then by lineage. Lineage is shown at the top of each heatmap. FIG. 11a, Heatmap for MamA motifs. Due to the large number of MamA motif sites, those with the configuration like those shown to affect transcriptional response to hypoxia (blue pop-out) and those within a region we observed to have a high density of hypervariable sites (red pop-out). Color of axis labels highlight motif sites shown by Shell and colleagues to affect transcriptional response to hypoxia (blue) and motif sites hypervariable across isolates with active MamA (red). FIG. 11b, Similar to the heatmap in FIG. 11a, but for HsdM motif sites. All common motif sites within 50 bp upstream of a TSS are shown. “Putative SFBS-overlapping sites” are those with an analogous configuration with the −10 promoter element shown by Shell & colleagues to affect transcription, but overlapping the end of the SFBS distal to the TSS, rather than the proximal end. “Partner sites” are loci at the position that includes the palindromic partners of putative SFBS-overlapping sites. Isolates with convergent methylation levels at a subset of notable loci despite having divergent HsdM genotypes and belonging to different lineages are indicated by asterisks (*).
FIG. 12A-F: Framework for a diagnostic device from sequencing kinetics. A method of classifying samples based on sequencing kinetics is depicted. FIG. 12A-C use real sequencing kinetics and resistance phenotype data from Mycobacterium tuberculosis and Mycobacterium africanum, demonstrating proof-of-concept for this particular application, and exemplifying the general method. FIG. 12D-F are conceptual depictions of the remaining steps comprising the diagnostic/clinical decision support tool. The example demonstrated in the depiction uses DNA methylation as the base modification with a kinetic signature, Mycobacterium tuberculosis/Mycobacterium africanum as the organism of interest, and resistance versus susceptibility to drug treatment and the phenotype to be classified. This method assumes a DNA sequence context (“motif”) that is preferentially modified is known. In the example, it is the known motif specificities for the MamA, HsdM, and MamB DNA methyltransferases of Mycobacterium tuberculosis. FIG. 12A) Identifying hypervariable motif sites. Kinetic variability is defined as the standard deviation (SD) of log 2 of the inter-pulse duration ratio (IPD ratio) at a given locus across samples. Ideally, a modification expected to be invariably present is used to define the parameters of how sequencing kinetics distribute at a given motif in the in the absence biologically selected unmodified sites. If no such site is known for the species of interest, the parameters can be estimated using the observed variability at such a motif (nearly invariably modified) in a different species with the same modification. In the example shown, expected kinetic variation across motif sites was modelled by motif sites of the M. tuberculosis MamB methyltransferase, which is expected to be nearly invariably modified, since exposed motifs would be cleaved by its cognate restriction endonuclease. Data from 93 isolates are included for HsdM, MamA, or MamB MTases whenever active in the isolate. Position (x-axis) in a representative genome and variability (SD of log 2(IPD Ratio) across isolates) in sequencing kinetics across isolates with active MTase (y-axis) at common motif sites (present in 75 or more isolates). Motif sites within three SD of the mean for MamB motifs are grey, and the outliers (>3 SD from mean) are highlighted in red. CDSs within which each of the top 10 most variable sites for each MTase occur are labelled, along with their palindromic partner motif site. FIG. 12B) Manhattan plot of the significance of association (corrected for multiple hypotheses using the FDR method, Benjamini-Hochberg) between methylated fraction of hypervariable motif sites identified in (A) and resistance phenotypes for 7 common anti-TB drugs, and eXtensively Drug Resistant phenotype (XDR). Shades of non-hits are alternated between associated drugs to distinguish from one another while hits (FDR <0.01) are colored blue. FIG. 12C) Correlation coefficients between all loci significantly associated with to at least one resistance phenotype (from FIG. 12B). Mutually correlated motif sites provide potential pairs for discriminatory fragmentation of DNA by restriction endonuclease digest, since it takes cleavage of two sites to excise a DNA fragment from its native chromosome. Mutually uncorrelated motif sites can be combined to additively inform resistance phenotype increasing predictive power. FIG. 12D) DNA extracted from M. tuberculosis complex bacteria directly from sputum or following culture is exposed to saturating levels of restriction endonuclease with the same specificity as the methyltransferase enzyme. FIG. 12E) Example of DNA fragment length quantification method using gel electrophoresis. The length travelled by fragments depends on their molecular weight and in turn, number of bp, from which one can ascertain which two unmethylated loci were frequently cut the REase, and hence unmethylated in the sample. FIG. 12F) Additively informative motif sites identified in FIG. 12C are optionally amplified with PCR or similar method, and their methylated fraction estimated. A different set of additively informative markers are assayed in parallel and their information combined to estimate the probability of resistance to each drug (or other desired phenotype associated with base modification status), and then output as a report in plain language for clinicians and laboratory technicians with potential calls of “susceptible”, “resistant”, or “heteroresistant”. Alternative classifications for other associated phenotypes could be reported similarly.
FIG. 13. MamB mutations mapped to annotated functional domains and predicted 3D structure. Mapping of mutations in mamB, and their effect on methyltransferase (MTase) function, at (a) primary, (b) secondary, and (c) tertiary levels of abstraction. Sequences from the assemblies examined in this study were drawn from the East Asian (EAS), Indo-oceanic (TO), and Euro-American (EAM) lineages, while those of the two ancestral mycobacteria, Mycobacterium bovis and Mycobacterium microti were obtained from a recent publication by Zhu and colleagues³. Nucleotide mamB Amino Acid sequences from these genomes were aligned using Tcoffee, with MTase functionality (inferred by methylation status of its motifs via SMRT-sequencing) indicated, and variants with respect to functional wild-type amino acid sequences mapped in the context of annotated domains from InterPro. These domains and mutations were in turn mapped (colors are preserved from B to the left structure of C) onto the predicted structure by RaptorX. The combination of well-curated functional annotation for this enzyme and the kinetic capabilities of SMRT-sequencing allow high-confidence hypothesis generation (in terms mutation-function inference) with resolution at the genomic, structural, and functional levels.

### Hypervariable Promoter Methylation Across Isolates Suggests Epigenetic Selection In Vitro.

Next, we cross-checked the hypervariable motif sites with promoter motif sites to identify sites of potential differential epigenetic regulation in vitro. Promoters of fifteen genes harbored hypervariable motif sites (FIG. 7d), with most (11/15) hypervariable in both palindromic sites. Hypervariable methylation across isolates could arise from either (i) highly variable degrees of methylation across isolates, or (ii) a bimodal distribution of hypomethylated sites and fully or mostly methylated sites (FIG. 17A). In prokaryotes, hypomethylation at both motif sites is a signature of epigenetic regulation, highlighting these eleven genes as putative epigenetically regulated sites. These sites might be selected differentially in vitro across the MTBC, or harbor genetic differences in DNA-binding proteins that compete with MTase, as we observed at cobK 304 (FIG. 6).

Seven motif sites comprise a cluster of hypervariable sites in the spacer between the −10 and −35 elements (19-24 bp range, FIG. 11a) of promoters harboring MamA motif sites. While this region does not overlap with sigma factor binding sites, transcriptional effectors commonly bind here to tune gene expression, providing a candidate mechanism driving the differences between strains. No MamB promoter motif sites were hypervariable (FIG. 7d), consistent with a classic RM-system without regulatory roles, once again contrasting with the signatures of gene regulation present at orphan MTase sites.

HsdM promoter methylation is associated with transcription levels of downstream genes. Notably, Rv1813c is hypervariable and has a motif site 11 bp upstream of its TSS, overlapping a SigA SFBS. Rv1813c was recently reported to be significantly under-expressed following AhsdM, but the authors did not identify the SigA overlap with this motif site in the Rv1813c promoter¹⁴. This discovery prompted us to re-evaluate the ΔhsdM differential expression results recently reported to have no direct influence on transcription at methylated promoters. In that work, the authors defined “differentially expressed” genes using thresholds on both significance (adjusted p-value ≤0.05) and magnitude (|log 2−foldchange|≥1). Since we are interested in the mechanism (Does HsdM promoter methylation have the capacity to influence transcription?) rather than the magnitude of its effect, we defined differentially expressed genes according only to significance. With these criteria, 310 genes (FIG. 24) were differentially expressed between hsdM_{WT} and ΔhsdM (ΔhsdM-DE). Genes with HsdM motif sites in their promoters (n=11) were significantly enriched (p-value=0.000215, OR=4.47, CI: 1.99-9.37, two-tailed Fisher's Exact) among ΔhsdM-DE genes (FIG. 7e). Therefore, we conclude that HsdM promoter motifs are associated with expression change following HsdM knockout, though the magnitude of the effect is subtle in standard media. We hypothesize that the magnitude of this effect conditionally increases through interaction with transcriptional effectors under other conditions.

FIG. 24 illustrates data supporting that knocking out the Methyltransferase MamC (referred to at times as HsdM) has a direct effect on transcription of genes with promoters methylated by MamC, and the figure illustrates sites that are differentially expressed upon MamC knockout that have promoters with MamC methylation sites.

Nine of these 11 ΔhsdM-DE genes with HsdM promoter motifs overlapped with the −10 promoter element (FIG. 7e), suggesting a direct effect on transcription analogous to the configuration for MamA previously described³. Within these nine are 3 of the 4 genes with hypervariable HsdM methylation in promoter motifs, suggesting that differential selection on the methylome during growth in vitro manifests at the transcriptional level.

### Promoter Methylation Implicates Mediators of Clinically Important Phenotypes

Our discovery of intercellular mosaic methylation, consistent hypomethylation, and widespread promoter methylation reveal multiple mechanisms for epigenetic gene regulation. To determine what processes and phenotypes are potentially regulated by these mechanisms, we examined the functional annotations of genes with methylated promoters and hypomethylated sites. From this examination emerged genes involved in host-lipid metabolism, drug resistance, metal ion homeostasis, and key regulators (Table 2, see FIG. 9).

Host-derived fatty acids and cholesterol are favored carbon sources for M. tuberculosis in macrophage³⁰. From host lipids, M. tuberculosis can generate energy, fuel central carbon metabolism, and synthesize cell wall components. Promoter MTase motifs and hypomethylated motifs fell in genes required to acquire these host lipids, dictate their metabolic fate, and detoxify intermediates generated during their utilization (Table 2, see FIG. 9). Several of these genes play crucial roles in regulating metabolic shifts in vivo. One such gene, ramB³¹, mediates the glyoxylate shunt³² through regulation of isocitrate lyase (Icl1), an enzyme that helps mitigate oxidative stress, effectively use different carbon substrates, and confers broad drug tolerance³³. Icl1 also functions as a methylisocitrate lyase that catalyzes the final step of the energy-generating methylcitrate cycle and serves as a propionyl-CoA sink³⁰. This multiplicity of roles for RamB's regulatory target highlight methylation status of its promoter as a potential epigenetic switch with cascading effects on lipid metabolism. Triacylglycerol (TAG) is synthesized primarily from host-derived free fatty acids by an enzyme encoded by tgs1³⁴, which has MTase motif sites in its promoter. TAG reduces oxidative stress incurred by free fatty acids by diverting flux away from the energy-generating TCA cycle³⁵ and inducing a dormant phenotype³⁶. TAG accumulates in vivo and serves as a carbon and energy reservoir³⁷ that can be utilized during dormancy. Also connected to dormancy is glpX, which encodes the rate-limiting enzyme of gluconeogenesis. Gluconeogenesis generates energy during dormancy, keeping metabolic and homeostatic processes running³⁸. Methylation of tgs1, glpX, and similar genes may mediate metabolic changes during transition into dormancy and survival in the dormant state.

Frequently hypomethylated genes accE5, bioB, and cobK (Table 1, see FIG. 8) are required for diverting odd-chain fatty acid flux down the methylmalonyl pathway (MMP). Dissipation through the MMP is preceded by propionyl-CoA conversion to methylmalonyl-CoA by a three-unit propionyl-coenzyme A carboxylase complex³⁹ one unit of which is encoded by accE5. bioB catalyzes the last biosynthetic step for producing biotin, a necessary cofactor for conversion to methylmalonyl-CoA⁴⁰. Once converted, methylmalonyl-CoA is either synthesized into virulence lipids, or proceeds down MMP if the necessary cofactor vitamin B12 is present³⁰. cobK harbors a frequently hypomethylated palindromic pair of motif sites (Table 1, see FIG. 8) and is required for synthesis of Vitamin B12⁴¹. B12 is required for the MMP yet is absent from standard media³⁰.

Rather than generating energy through the TCA cycle, methylmalonyl-CoA intermediates can also be assembled into virulence lipids. This alternative pathway is mediated by pks genes⁴², which harbor hypomethylated motif sites (pks6 and pks9, Table 1, see FIG. 8) and a promoter motif site (pks15). pks15 encodes a polyketide synthase in East-Asian isolates that is absent from H37Rv⁴³. This enzyme is required to synthesize phenolic glycolipids⁴⁴, which confer hypervirulence to the W-Beijing sublineage⁴⁵. Similarly, mptA synthesizes mycobacterial glycolipids from these intermediates⁴⁶, and harbors a hypomethylated site. The promoter methylated Rv3779 and hypomethylated treZ each synthesize virulence lipid components (mannosides⁴⁷ and trehalose⁴⁸, respectively).

MTase motifs reside within promoters of genes mediating both intrinsic and acquired drug resistance (Table 2, see FIG. 9). These genes mediate resistance through gene regulation (whiB7-controlled expression of eis, tap, and Rv1473 and raaS-controlled expression of Rv1218c and Rv1217c), drug efflux (drrA, iniA, Rv3728, and efflux-targets of whiB7 and RaaS), and other mechanisms (mshC, mshD, Rv3050c, glf, and gyrB). Promoters of genes implicated in efflux-driven non-genetic persistent mechanisms harbor MTase motif sites. Promoters of drrA and iniA harbor MTase motifs and are seminal examples of phenotypic persistence driven by efflux pump overexpression⁴⁹. Additionally, transcriptional regulator whiB7, whose expression has been demonstrated previously to be modulated by methylation status, controls expression of efflux and other intrinsic resistance genes (Table 2, see FIG. 9)⁵⁰.

Promoter methylation of these genes likely influences efflux pump activity and metabolic quiescence, two primary sources of phenotypic heterogeneity in persister cells⁵¹. Intercellular mosaic methylation may thus imbue some bacilli with methylation patterns that alter expression favorably for tolerating drug pressure. The epigenetically defined tolerant minority would then enable colony survival in fluctuating drug concentrations, buying time for genetic resistance mechanisms to emerge under prolonged pressure.

Curiously, promoter methylation patterns in RaaS (Rv1219c) converge between distant isolates (FIG. 5c, red arrows; FIG. 11b, asterisks). RaaS mediates intrinsic resistance to rifampicin and isoniazid by inducing drug efflux. Three East-Asian isolates with wild-type hsdM were hypomethylated at both palindromic ends of a motif site within the RaaS promoter, overlapping with a SigA motif. These East-Asian isolates were closely related (SNP distance 7-618), but the RaaS promoter was also hypomethylated in an M. africanum isolate distantly related to the East-Asian triplet (SNP distance <2,722). The M. africanum isolate did have a slight knockdown mutation (hsdM: K458N, E481A), but the majority of its HsdM motif sites were methylated (FIG. 2a, middle, FIG. 5b). This cross-phylogeny convergence suggests that factors external to MTase mutations can influence what methylation patterns emerge in the colony following in vitro growth. An alternative explanation is that each of the four strains could share a genetic factor responsible for this methylation anomaly, such as a transcription factor affecting promoter methylation as in the scenario described in FIG. 6. It is yet unclear whether this convergence affects drug tolerance.

### Table 2, Illustrated as FIG. 9:

Systems implicated at putative epigenetically modulated promoters and consistently hypomethylated sites. Genes implicated as epigenetically-regulated that are involved in clinically relevant processes. These genes mediate known intrinsic and acquired resistance and mechanisms, metabolism of host-derived lipids and flux through subsequent metabolic pathways, metal ion homeostasis.

The final implicated process is metal ion homeostasis. Cobalt (corA), magnesium (corA), copper (lpqS), and iron (mmpS4, higA, mbtJ, and hemN) homeostasis genes harbor methylated promoters (Table 2, see FIG. 9). Metal ion homeostasis is critical for in vivo niche adaptation and is phase-variable in other pathogens¹⁴. Metal ion availability differs between in vivo microenvironments, and M. tuberculosis must respond to these dynamic concentrations to maintain homeostasis⁵²⁻⁵³. Cobalt is required for de novo biosynthesis of vitamin B12, a key factor in methionine biosynthesis and the methylmalonyl pathway⁴¹. Magnesium acts as a cofactor for numerous reactions, and copper-response pathways are required for full virulence⁵⁴. Heterogeneous expression of genes dictating metal ion homeostasis might prime subpopulations for rapid adaptation upon introduction to a new microenvironment and may have roles in drug tolerance^{53,55,56}.

### Discussion

Here, we leverage third-generation sequencing technology to investigate an DNA methylation at single-nucleotide resolution, an underexplored source of variation among the MTBC. We assembled, annotated, and compared DNA methylomes of 93 clinical isolates spanning all seven MTBC lineages. This comprehensive survey clarified the diversity and function of MTase variants across the MTBC and produced several novel findings. First, the methylome of virulent M. tuberculosis type strain H37Rv is dissimilar to methylomes of recent clinical isolates. Second, promoter methylation is abundant, occurs in a conserved configuration with sigma factor binding sites, and is upstream of key mediators of drug resistance and other clinically important phenotypes. Third, the methylomes of genetically distant isolates converged in several cases, exposing the limitations of using genetic distance alone as a proxy for phenotypic similarity in M. tuberculosis. Fourth, intracellular stochastic methylation in individual cells creates intercellular mosaic methylation within M. tuberculosis colonies. This intercellular mosaic methylation is genetically driven in some isolates and can be induced by methionine starvation in H37Rv. Finally, our re-analysis of RNAseq data in wild-type versus HsdM-knockout demonstrates direct transcriptional influence by HsdM promoter methylation (FIG. 7e, FIG. 24), providing a potential mechanism for mosaic methylation to produce phenotypic heterogeneity. This previously undescribed mechanism offers new explanatory hypotheses for long-standing mysteries in M. tuberculosis phenotypic plasticity and evolution. More broadly, these findings highlight comparative methylomics as a fertile and underexplored avenue to understand microbial diversity, gene regulation, and evolution that complements genetic and phenotypic analysis. This global isolate set of all seven MTBC lineages revealed the convergence of methylomes across genetically distant strains and highlighted the rarity of virulent M. tuberculosis type strain H37Rv's methylome. Many isolates from different lineages had the same MTase activity profile, while many isolates from the same lineage had different MTase activity profiles (FIG. 5c). In one curious case, isolates from distant lineages also shared site specific hypomethylation (FIG. 5c, FIG. 11b). These findings contradict previous reports that M. tuberculosis methylomes were lineage specific⁴. As differentially methylated sites outnumbered SNPs between many isolates (FIG. 3), shared methylomes may result in similar phenotypes between genetically distant strains.

Kinetics data analysis of all known MTase motif sites revealed knockdown MTase mutations that inspired subsequent heterogeneity analysis. The heterogeneity analysis identified four unique MTase variants that conferred intermediate IPD ratios at all target motif sites, suggesting epigenetic heterogeneity. SMALR¹⁵ confirmed this heterogeneity in mamA:E270A and mamA:G152S isolates, and characterized the phenomenon as intracellular stochastic methylation, rather than phase variant MTase knockout. In stochastic methylation, the methylation status of each MTase target site varies independently between cells. The resulting subpopulations carry diverse combinations of methylated and unmethylated sites, a phenomenon we have termed “intercellular mosaic methylation”. Further analysis demonstrated intercellular mosaic methylation occurs even with wild-type MTases, when under methionine starvation. This suggests nutritive stress may diversify phenotype through differential methylation patterns. Intercellular mosaic methylation appears to serve as an adaptive response and as a constitutive source of diversity in some isolates.

The most frequent variant associated with intercellular mosaic methylation, mamA:E270A, was ubiquitous among Beijing isolates, and may contribute to their global success. Intercellular mosaic methylation may confer an enhanced ability to colonize new hosts with diverse genetic background and immunities through varied modes of transmission. Indeed, methylated promoters are present in many genes linked to hallmarks of Beijing sublineage: facile dormancy induction⁷⁵, increased host-lipid utilization, TAG accumulation in aerobic environments⁷⁶, and increased synthesis of cell envelope components and virulence lipids⁷⁷ (Table 2, see FIG. 9). Some of these hallmarks have been attributed to genetic factors, such as mutations increasing basal expression of the DosR-regulon⁷⁶. Yet gaps remain in our understanding of what differentiates the Beijing sublineage from others, which intercellular mosaic methylation may ultimately explain. This potential role of constitutive mosaicism in Beijing's success could provide new leads to therapeutics targeting intercellular mosaic methylation, or diagnostics associating methylation status of motif sites to clinically important phenotypes.

M. tuberculosis has evolved diverse transcription factors that invoke transcriptional programs to promote survival in microenvironments throughout its lifecycle. Yet transcriptional responses to environmental changes are delayed³⁹, begging the question: How does M. tuberculosis survive before these transcriptional responses take hold? Our findings support a model in which intercellular mosaic methylation imbues some bacilli with methylation patterns that influence transcription favorably for survival in a particular set of conditions. Then, upon appearance of this set of conditions, subpopulations with advantageous methylation patterns survive long enough for transcriptional reconfiguration to manifest through genetically encoded transcriptional programs. This model of intercellular mosaic methylation-driven heterogeneity is consistent with prior observations of M. tuberculosis “persister cells”⁴⁰, minority groups that are pre-adapted to tolerate initial exposure to macrophage⁴¹ and drug pressure⁴², by entering dormancy⁴³ or activating efflux pumps^{44,45}. Reconciling observations of persister cells with our described model requires MTase motifs to affect transcription of the genes mediating persistence, and a plausible mechanism for DNA methylation to influence transcription. We find evidence for both these requirements.

Promoter methylation motifs implicate dormancy, antimicrobial resistance, and metal ion homeostasis as processes regulated in part by DNA methylation. Rv1813c and hrp1 are especially highly expressed members of the M. tuberculosis dormancy regulon, and hypervariable across isolates with active MTase (FIG. 20). While the function of Rv1813c is unknown, it was one of four antigens formulated into a vaccine designed for boosting efficacy of the BCG vaccine³⁷, and ΔRv1813c mutants show reduced immune response and diminished bacterial survival in a mouse model of tuberculosis³⁸. Rv1813c expression decreased over two-fold in ΔhsdM (FIG. 7e). Together, these observations point to methylation in the Rv1813c promoter as an epigenetic determinant of clinically important phenotypes. Overexpression of hrp1 in M. smegmatis improves survival in ex vivo assays in macrophage and murine tissue, increases cell necrosis, and induces key immune effectors⁴⁶.

Intercellular mosaic methylation-driven heterogeneity also implicates the metabolic side of dormancy. Transcriptional influence by the −10 promoter element motif site of ramB⁴⁷ is an intriguing candidate for future investigation. RamB mediates the glyoxylate shunt⁴⁸ through transcriptional regulation of Isocitrate lyase (Icl1), a key player in central metabolism, handling oxidative stress, and tolerating antimicrobials⁴⁹. Hypervariable, ΔhsdM-DE promoter methylation of glpX also implicates dormancy metabolism. Its product, (GlpX) encodes the rate-limiting enzyme of gluconeogenesis, the pathway through which dormant M. tuberculosis furnishes energy⁵⁰.

In vivo, the human immune system imposes its own dynamic selective pressure on M. tuberculosis, which remains incompletely understood. Several of the better characterized immune pressures destroy a majority of bacilli, while a minority subpopulation survives. For example, minor subpopulations of M. tuberculosis successfully rupture host phagosomes, allowing access to the host cytoplasm⁸¹. Intercellular mosaic methylation may play a role in establishing this heterogeneity, allowing the pathogen to employ multiple strategies simultaneously to combat the host immune system.

Multi-omic integration with annotated and assembled whole methylomes revealed widespread epigenetic gene regulation through promoter methylation. MTase target motifs frequently coincided with classical promoter elements (FIGS. 7 and 11), strongly suggesting transcriptional influence. These key promoter elements guide the formation of transcription initiation complexes⁸². DNA methylation alters biophysical properties that tune promoter strength, including DNA melting temperature⁸³ and bending DNA near the −10 promoter element during open complex formation⁸⁴. Prior work demonstrated MamA knockout in H37Rv caused widespread changes in transcription and downregulated the expression of four genes with MamA motifs in their promoters: Rv0102, Rv0142, conA, and whiB7⁷. Our work identified several dozen more genes with MTase motif sites at similar positions within their promoters (FIGS. 7 and 11; FIG. 23) and provided strong evidence that HsdM promoter methylation similarly influences transcriptional (FIG. 7e).

Several genes with methylated promoters are deeply linked to drug resistance, host lipid metabolism, persister cell formation, and key metabolic shifts in vivo. This linkage has two key implications. First, these genes, and their associated metabolic processes, appear to be epigenetically regulated through DNA methylation status. Second, through intercellular mosaic methylation, colonies gain access to a broader range of phenotypes that are not immutably fixed through chromosomal mutation. Rather the methylomes are passed vertically, through semi-heritable epigenetic inheritance. This may enhance colony robustness against changing conditions while preserving a majority subpopulation that is phenotypically adapted to current conditions. The notion that that intercellular mosaic methylation confers an adaptive advantage is supported by its convergence across three lineages, and its emergence following methionine starvation in ΔmetA mutants.

We cannot extrapolate directly from the DNA methylation patterns we report here to what occurs during infection. Sequencing kinetics are measured from DNA extracted after extensive culturing, during which any methylomic adaptation to the host environment would have presumably been erased. Directly sequencing from sputum is ideal to assay DNA methylation patterns in vivo, but SMRT-sequencing requires large quantities of DNA, necessitating in vitro culturing, as DNA amplification erases epigenetic markings. In the absence of lower DNA input requirements, in vitro studies under host-like conditions (e.g. hypoxia, host-lipids as carbon source) can reveal context-dependent selection of methylation patterns, and time-course serial sequencing could inform us of the dynamics methylomic adaptation their selection. Coupling these sequencing studies with transcriptomic, proteomic, and phenotypic assays could clarify how effects of DNA methylation manifests in gene expression and phenotypically.

Transcriptional responses in M. tuberculosis are mediated by numerous effectors of transcription, many of which are not constitutively expressed. Interaction with additional transcriptional effectors has been described to interact with DNA methylation in other bacterial species⁸⁰ to modulate transcription. The cluster of hypervariable motif sites in the spacer between −10 and −35 promoter isolates in MamA-methylated promoters (FIG. 11a) and the hypervariable ΔhsdM-DE motif sites in this region in the Rv1219c (RaaS) promoter (FIG. 7e) support this idea.

The large set (n=351) of hypervariable loci (FIG. 20) we observed methylation patterns are either differentially selected in vitro, or indirect effects of a related process differential between strains, such as TF-binding. Remarkably, methylation patterns of three East Asian isolates converged a genetically distant M. africanum isolate with discordant HsdM alleles, demonstrating convergent epigenomic selection in vitro. These sites included Ahso/M-DE persistence (RaaS) and dormancy (Rv1813c and glpX) genes, suggesting this convergent methylomic adaptation has important phenotypic consequences.

Our findings raise important questions. First, are MTase genotypes with wild-type activity in rich media similarly active in vivo or do they exhibit intercellular mosaic methylation under constraints such as cofactor limitation or DNA accessibility? The intercellular mosaic methylation observed in methionine-deprived ΔmetA mutants (FIG. 4) demonstrates that intercellular mosaic methylation can be induced by nutrient deprivation, but it is unclear what other conditions elicit intercellular mosaic methylation. Methionine is directly linked to DNA methylation by its requirement of S-adenosyl methionine (SAM) as the methyl group donor. Since methionine starvation induces intercellular mosaic methylation, it can be argued that the effect could be extrapolated to other nutrient restrictions that limit flux through the adenine methylation reaction, such as precursor metabolites for SAM synthesis or trace metal ion cofactors required for SAM biosynthesis and adenine methylation. While direct sequencing from sputum is ideal to assay DNA methylation patterns in vivo, current SMRT-sequencing technology makes this difficult. SMRT-sequencing requires large quantities of DNA, necessitating in vitro culturing or DNA amplification, which erases epigenetic markings. Until DNA input requirements improve, in vitro studies under host-like conditions (e.g. hypoxia, host-lipids as carbon source) may shed light on context-dependent intercellular mosaic methylation.

Methylome rearrangement dynamics are another key question. Under the prevailing view, demethylation does not occur in bacteria (though base excision repair might offer a demethylation mechanism⁸⁵). Under this view, demethylation can occur only between generations, through a lack of re-methylation on the nascent strand following replication. Accurate modeling of how the methylome changes within and across generations requires greater knowledge of methyltransferase activity throughout the cell-cycle. The nature of these dynamics has key implications. If M. tuberculosis DNA MTase are active throughout the cell-cycle DNA methylation could mediate acute responses to environmental cues. Alternatively, if MTase expression is restricted to a particular part of the cycle as in E. coli dam⁸⁶, DNA methylation status can only be selected upon. Comparative methylomics combining SMRT-sequencing kinetics analysis, cell-cycle coordination⁸⁷, and MTase activity probes would answer these questions, especially if employed across multiple conditions.

Our promoter methylation reports different results and opposing conclusions to those reached in a recent analysis¹⁴ on the role of HsdM in regulating promoter strength. While they conclude that “methylation seems to play a minimal role in shaping in-vitro gene expression”, integrating our identified promoter MTase motif sites with data their ΔhsdM RNAseq experiment shows a clear association between HsdM promoter methylation and in vitro gene expression (FIG. 7e). We believe differences in approach drove our disparate conclusions. For their analysis, Chiner-Oms and colleagues relied on reference-mapping, a single source of TSSs, and focused on SigA motifs to identify promoter MTase motifs¹⁴. In contrast, we transferred TSS annotations from two M. tuberculosis transcriptomic studies to contiguous regions of finished de novo assemblies, and scanned for SigA-SigM SFBS motifs³⁴, and promoters lacking known SFBSs. These differences explain why our analysis captured the association between HsdM methylation and expression of downstream genes.

Our approach differed from prior analyses of MTBC methylomes and key to our findings:
(i) Analyzing sequencing kinetics at all motif sites kinetics in every isolate.
(ii) Using finished assemblies comparative genomics of MTase alleles and regulatory elements.
(iii) Heterogeneity analysis.
(iv) Transferring CDS and TSS annotations from an extensively studied reference strain.

While the relative clonality of the MTBC made annotation transfer straight-forward, species with more dynamic genomes may present additional challenges. We recommend similar approaches for future large-scale, intra-species comparative and functional methylomics studies in prokaryotes.

The data and isolate set described in this work can help answer these questions and others regarding the role of DNA methylation in M. tuberculosis. This isolate set comprises all seven lineages of the MTBC and have finished, annotated genomes and methylomes. Future experiments with these isolates can show the effects of methylomic differences on phenotype and adaptive capacity.

This work extends upon recent characterization of MTase motifs, their DNA methyltransferases³, and their capacity to modulate transcription⁷ in M. tuberculosis. We find epigenetic diversity in M. tuberculosis and evidence that it manifests as clinically important phenotypic diversity. Knockdown and knockout mutations emerge repeatedly in DNA methyltransferases, punctuating M. tuberculosis evolution with sudden change at several thousand sites. Stereotyped promoter methylation configurations indicate widespread epigenetic regulation in M. tuberculosis. These findings demonstrate DNA methylation as a fundamental source of diversity that potentially explains the discord between the limited genetic variation reported in M. tuberculosis and its observed capacity for phenotypic adaptation. More broadly, the discovery of intercellular mosaic methylation in M. tuberculosis reveals that the pathogen forms diverse methylation patterns, conferring a continuum of semi-heritable⁸⁸ phenotypes to be selected into epigenetic lineages. This phenomenon provides a new mechanism of phenotypic plasticity in pathogens and opens the door to new therapeutic angles—and challenges—for tuberculosis control.

### Methods

Code availability. All custom code used to for this analysis are publicly available at: https://gitlab.com/LPCDRP.
Isolate acquisition and inclusion criteria. M. tuberculosis colonies were isolated from sputa of tuberculosis patients in the five sites (FIG. 1), comprising multiple members of four M. tuberculosis lineages and a single isolate belonging to Lineage 7. These isolates originated from Hinduja National Hospital (PDHNH) in Mumbai, India; Phthisiopneumology Institute (PPI) in Chisinau, Moldova; Tropical Disease Foundation (TDF) in Manila, the Philippines; The National Health Laboratory Service of South Africa (NHLS) in Johannesburg, South Africa; and the Supranational Reference Laboratory in Stockholm, Sweden. Of these isolates, 94 passed assembly quality control. An additional 8 isolates failed our methylome pipeline (2 isolates had multiple contigs and 3 isolates had position inconsistencies between their kinetics data and final FASTA sequence file), leaving 86 clinical M. tuberculosis isolates. We supplemented these isolates by downloading publicly available SMRT sequencing reads, including 6 clinical M. tuberculosis isolates, 4 M. africanum isolates, a triplicate run of virulent type strain H37Rv, and triplicate samples of a metA knockout strain of H37Rv following five days of methionine starvation. Two of these isolates failed our assembly QC, and 3 failed our methylome pipeline (multiple contigs). Finally, we included technical replicate control runs of avirulent reference strain H37Ra reported in a previous paper⁸⁹. In total, our pipeline completed analysis on 101 samples, including 90 clinical M. tuberculosis isolates, 3 M. africanum isolates, and 8 runs of reference strains and knockouts. (BioProject Nos: PRJNA555636, PRJNA329548, PRJEB8783)
Sample preparation and extraction. Samples prepared and extracted in Sweden at the Supranational Reference Laboratory, in Stockholm were performed as previously described. All samples were streaked for isolation using standard microbiological methods, after which well separated colonies were selected, emulsified, and sub-cultured on Lowenstein-Jensen slants and Middlebrooks 7H11 plates, where they were incubated until growth of a full bacterial lawn. DNA was extracted using Genomic-tips (Qiagen Inc., Germantown, Md.) following the manufacturer's sample preparation and lysis protocol for bacteria with the following modifications. Each culture was harvested directly into buffer B1/RNAse solution, homogenized by vigorous vortex mixing and inactivated at 80° C. for 1 hour. Lysozyme was added and incubated at 37° C. for 30 minutes followed by the addition of proteinase K and further incubation at 37° C. for an additional 60 minutes. Buffer B2 was added and the mixture was incubated overnight at 50° C. The remainder of the Genomic-tip protocol was carried out exactly as described by the manufacturer. DNA purity and concentration were analyzed on a Nanodrop 1000 (Thermo Scientific, Waltham, Mass., USA). DNA sequencing. DNA sequencing was performed at the Institute for Genomic Medicine at the University of California, San Diego. DNA libraries for PacBio (Pacific Biosciences, Melon Park, Calif.) were prepared using PacBio's DNA Template Prep Kit with no follow-up PCR amplification. Briefly, sheared DNA was end repaired, and hairpin adapters were ligated using T4 DNA ligase. Incompletely formed SMRTbell templates were degraded with a combination of Exonuclease III and Exonuclease VII. The resulting DNA templates were purified using SPRI magnetic beads (AMPure, Agencourt Bioscience, Beverly, Mass.) and annealed to a two-fold molar excess of a sequencing primer that specifically bound to the single-stranded loop region of the hairpin adapters. SMRTbell templates were subjected to standard SMRT sequencing using an engineered phi29 DNA polymerase on the PacBio RS system according to manufacturer's protocol.
Genome assembly. For isolates that were sequenced on multiple SMRT cells, all SMRT cell raw reads were combined and assembled with either HGAP2⁹⁰ or canu⁹¹ with default parameters. Circularization was then performed to confirm a circular genome using minimus2 from amos or circlator⁹². Gene dnaA was set as the first gene in each genome. Iterative rounds of consensus polishing using BLASR⁹³ and Quiver were executed three times. Default parameters were used except max coverage was set to 1000 for Quiver. Genomes failed assembly quality control if they could not be circularized, if their consensus polishing resulted in five or more variants after three iterations, or if PBHoney⁹⁴ detected a structural variant in the assembly supported by at least 10% of the reads. PBHoney was run with default parameters.
Analysis of sequencing kinetics. To determine the inter pulse duration (IPD) ratio at each nucleotide in each isolate, we ran Single Molecule Real Time (SMRT) analysis with the Base Modification Detection with Motif Finding protocol with default parameters. A custom R script then scanned the FASTA sequence file of each isolate for matches to the MTase target motifs previously characterized in M. tuberculosis³, then extracted the IPD ratio of the targeted adenine in each matching site from the Base Modification output. These IPD ratios were then log transformed to produce a normal distribution, and standardized by subtracting the mean IPD ratio (also log transformed) of all adenines outside of MTase motifs in the isolate. Additional custom R scripts plotted the distribution of processed IPD ratios in each isolate to characterize their MTase activity (FIG. 2), identified hypervariable MTase sites across isolates (FIG. 5), and performed further analysis as described.
Lineage Determination. For isolates that were re-sequenced, lineage information was obtained by inserting the MIRU-VNTR and spoligotype patterns determined previously³⁹ into TBInsight⁹⁵. For all other genomes, a custom script, MiruHero (https://gitlab.com/LPCDRP/miru-hero), determined lineage.
Genome annotation. RATT transferred Transcriptional Start Sites (TSS) from our curated H37Rv annotation. These TSS were originally determined experimentally in the H37Rv strain by Cortes et al²⁵ and Shell et al²⁴, and merged into the H37Rv an in-house annotation with custom scripts.
Methylome annotation. Using the annotated genome of each isolate, we annotated their MTase motif sites with a custom python script, which recorded the relative position and gene name of any CDS or TSS features overlapping or neighboring each MTase motif site. To track MTase motif sites across isolates, each MTase motif site was assigned a locus tag based on the nearest CDS boundary.
Methylome annotation. Using the annotated genome of each isolate, we annotated their MTase motif sites with a custom python script, which recorded the relative position and gene name of any CDS or TSS features overlapping or neighboring each MTase motif site. To track MTase motif sites across isolates, each MTase motif site was assigned a locus tag based on the nearest CDS boundary.

Separately, we also annotated the MTase motif sites using RATT alone, with the curated H37Rv reference annotation. Using RATT without Prokka and the rest of the AnnoTUB pipeline left many genomic regions unannotated, but more consistently annotated MTase motif sites near hypervariable genes such as PE_PGRS54 and PE_PGRS57. In many isolates, MTase motif sites near these genes were assigned different locus tags when using AnnoTUB genome annotations, as AnnoTUB labeled PE_PGRS54 and PE_PGRS57 as new genes in these isolates because they lacked 95% sequence identity.

MTase genotyping. To determine the genotype of the MTase genes mamA (Rv3263), mamB (Rv2024c), and hsdM (Rv2756c)/hsdS (Rv2761c) in each isolate, first eggNOG-mapper⁹⁸ identified these genes in each clinical isolate, through homology to these genes in annotated reference genome of M. tuberculosis type strain H37Rv. However because MamB and HsdM are inactive in the H37Rv strain³, we did not use the H37Rv genes as the wild-type allele. Instead, sequencing kinetics and the previously characterized target motifs were used to determine which isolates had active copies of each MTase gene, and the most common sequence among active isolates was defined as the wild-type sequence. To call variants in these genes using these wild-type sequences, BLASTn then aligned the wilt type sequences against all genes predicted in each isolate by Prodigal⁹⁹. Each matching nucleotide sequence was translated into an amino acid sequence using transeq (EMBOSS 6.6.0.0, available online at www.ebi.ac.uk/Tools/emboss/transeq/index.html) to obtain nonsynonymous variants and truncations. The amino acid sequences were then aligned using MAFFT¹⁰⁰ v7.205 with the—clustalout option, and a custom script converted the alignment to a genotype.
Variant Calling for building phylogenies dnadiff¹⁰¹ (v1.3) aligned each assembled genome to M. tuberculosis H37Rv (NC 000962.3) and call SNPs and small indels with default parameters. A custom Perl script converted the out.snps from dnadiff into a VCF v4.0 file and Variant Effect Predictor¹⁰² (v87) determined the consequence of each variant.

### For MTase Genotyping:

Phylogeny construction and mapping of MTase genotypes. First an alignment of concatenated variants was created using each isolate's VCF file. Then this alignment was used to create a maximum likelihood phylogenetic tree using RAxML¹⁰³ version 8.2, specifying a general time-reversible model of nucleotide evolution with 100 bootstrap replicates. The Interactive Tree of Life (iTOL) webtool¹⁰⁴ was used to visualize and map data to the tree, such as lineage and MTase genotypes.
Heterogeneous methylation analysis. SMALR¹⁵ requires a de novo assembled genome FASTA file and a cmp.h5 file with aligned reads, to extract the IPD data from each MTase target motif site within each read. We created a cmp.h5 for each isolate by aligning its reads to its assembled FASTA file using BLASR⁹³. We ran SMALR on each isolate with the SMp (single molecule, pooled distribution) argument. For MamA sites we set the motif to CTGGAG, the modified position within the motif to 5, and the minimum number of motif sites per read to 6. For MamB sites we set the motif to CACGCAG, the modified position to 6, and the motifs per read threshold to 5. From the SMALR output we used the native score of each read in place of SMp score. The SMp score can only be calculated if a PCR amplified control run of each isolate is provided. This substitution is susceptible to noise from local sequence contexts, but should still resolve differences between isolates and, per the authors of SMALR, it should still distinguish methylated and unmethylated components. We analyzed the distribution of native scores within each isolate for MamA and MamB sites using custom R scripts.
Identification of promoters. To identify MTase motif sites in gene promoters, a custom python script first scanned the surrounding sequence of each MTase motif site in each isolate for Sigma Factor Binding Sight (SFBS) motifs previously characterized in M. tuberculosis²⁶. If a SFBS match overlapped an MTase motif site, then the script checked if that SFBS match was the appropriate number of bases upstream from a TSS annotated in that isolate. For example, if the SFBS match was the −10 component of a SFBS, the script checked if a there was a TSS on the same strand with a genome position 8 to 12 bp downstream of the matching sequence. If the SFBS match was a −35 component of a SFBS, the script instead checked for a TSS between 30 and 40 bp downstream. MTase sites that met these criteria were labeled with the sigma factor type of their overlapping SFBS, their distance upstream of the TSS, and the gene name of the closest CDS downstream from the TSS. Since these criteria are rather conservative, more relaxed boundary thresholds were implemented for some of the promoter methylation analyses.
Reference-based differential methylation: In each in each clinical isolate we extracted all MTase motif sites that shared their loci with an MTase motif site in reference strain H37Rv, then counted the number of these sites with opposing methylation calls. These counts were then compared to the median SNP distance between each isolate and H37Rv (FIG. 3) calculated from the VCF variant file of each isolate.
Bayesian classification of base specific methylation status: Even within isolates with active MTase genotypes, not every base with an MTase target motif was methylated. To identify MTase motif sites with no base modification (hypomethylated sites) we took a Bayesian approach. In each isolate our custom R script estimated the distribution of normalized IPD ratios among unmodified bases by calculating the standard deviation and mean normalized IPD ratios of bases not within MTase motifs. The script then estimated the distribution of methylated bases by calculating the standard deviation and mean of bases targeted by MTase motifs. This estimate assumed that most bases targeted by MTase motifs were methylated, which held true in isolates with active MTase genotypes (FIG. 2a). For MTase motif site, the script calculated the conditional probability of the base belonging to either the modified or unmodified population, given its normalized IPD ratio and coverage. The script classified all bases more than nine times more likely to belong to the unmodified population as hypomethylated, all bases more than nine times more likely to belong to the modified population as methylated, and the remaining bases as indeterminate.

The coverage of each MTase site in each isolate was used to adjust the standard deviation of the distributions used to calculate its conditional probability, as bases with lower coverage have more variable IPD ratios (FIG. 14D). To perform this coverage adjustment, for each isolate we trained a model to estimate the expected standard deviation of any base given its coverage. After log 2 transforming and normalizing the IPD ratios of all bases in an isolate, the script calculated each base's number of standard deviations from the median normalized IPD ratio. Next, linear regression estimated the relationship between these standard deviations and the inverse coverage of each base. The resulting model estimated the standard deviation for each possible coverage value. When estimating the conditional probabilities of each MTase motif site, the code first calculated the mean and standard deviation of normalized IPD ratios in adenines within and without MTase motifs. It then multiplied these two standard deviations by the standard deviation predicted from the sequencing coverage at that MTase motif site. These adjusted standard deviations were then used to estimate the distribution of normalized IPD ratios, and calculate the conditional probability of the MTase motif site belonging to those distributions.

Conserved hypomethylation patterns. Using the Bayesian classification of each MTase motif target and the loci labeled by our methylome annotation pipeline, we searched for hypomethylated loci that occurred in multiple isolates. For each locus, a custom R script counted the number of isolates with that locus, including only isolates with active genotypes of the MTase targeting the locus. Our script also counted the number of these isolates in which the locus was hypomethylated. To estimate the significance of these findings, we used a cumulative binomial test, with the first count as the sample size and the second count as the number of successes. To find the probability of hypomethylation for each Bernoulli trial if hypomethylation occurred randomly, we calculated the total frequency of hypomethylation among MTase motif sites in active isolates. A separate per trial probability was calculated for MamA, MamB, and HsdM. The Bonferroni correction adjusted for multiple hypothesis testing, by dividing the significance threshold by the total number of unique loci in this study. No reference strains were included in the analysis of hypomethylated loci.
Transcription factor binding motif scanning. We searched for Transcription Factor (TF) binding motifs near hypomethylated bases using the command line motif scanner FIMO¹⁰⁵ version 4.12.0. For each hypomethylated locus in an MTase target motif, we extracted the sequence of 41 bases surrounding the locus in a randomly selected representative isolate (only isolates hypomethylated at that locus were chosen). The context sequences were combined into a multisequence FASTA file. Probability weight matrices of each TF binding motif were kindly provided by Minch and colleagues¹⁹, who derived them from a ChIP-Seq experiment on virulent M. tuberculosis type strain H37Rv. We then ran FIMO using each TF motif on the context FASTA file with a threshold p-value of 0.01. For comparison we also scanned for TF motifs in the context sequences of consistently methylated loci (consistently methylated loci here defined as loci present in at least 30 isolates and methylated in at least 95% of those isolates). Custom scripts then parsed the FIMO output files for each TF binding motif and counted the number of methylated loci and the number of hypomethylated loci matching each TF with a q-value of at least 0.1.
Proximal MTase motif search. For each MTase motif site in each isolate, we found neighboring MTase motif sites through a custom R script. The script found the nearest MTase motif either upstream or downstream from each MTase motif, and recorded the distance in bp.
Methylation anomalies. For each MTase, a custom R script found the set of MTase motif site loci present in at least 75 isolates. For each locus, summary statistics (mean and standard deviation) of mean log(IPD Ratio) were calculated exclusively from isolates with active MTases for each motif. The same was then performed to obtain median and standard deviation of mean log(IPD Ratio) for inactive isolates of each activity profile for each MTase. Hypervariable HsdM, MamA, and MamB motif sites were classified as those more than 3 S.D above the mean for MamB motif sites, since they had the fewest outliers (FIG. 5e) and are not an orphan MTase (FIG. 13).
RNA-Seq Re-Analysis and Integration. See Supplementary Table 9 from Chiner-Oms et al, Nature Communications vol 10, article no. 3994 (2019), see also https://www.nature.com/articles/s41467-019-11948-6 and merged with our annotated promoter for HsdM. A Benjamini-Hochberg adjusted p-value threshold of 0.05 was set as the criteria for being considered “differentially expressed”, using the column labelled “padj (BH)” from Supplementary Table 9 of Chiner-Oms et al. Two-sided Fisher's Exact Test was implemented in R to test for independence of HsdM promoter presence and Differentially methylated genes following HsdM Knockout. Genes were considered to have an HsdM promoter motif is the modified adenine was within 50 bp upstream of the TSS.

### Example 2: Intercellular Mosaic Methylation (IMM) is Distinct from Other Forms of Mosaic-Like DNA Methylation

Sequencing kinetics of MTase target motif sites indicated heterogeneous methylation in isolates with MTase variants mamAEroA, mamAG152s, and mamB_{K1033T} (see FIG. 4C and FIG. 4D; and FIG. 15B). Read-level kinetic analysis confirmed this heterogeneity, and characterized the phenomenon as intracellular stochastic methylation, rather than phase-variable methylation (FIG. 4G). Further heterogeneity analysis demonstrated that methionine starvation can induce intracellular stochastic methylation in isolates with wild-type MTase activity (FIG. 4E, FIG. 4F). In stochastic methylation, the methylation status of each MTase target site varies independently between cells (Beaulaurier et al., 2015). The resulting subpopulations thus carry diverse combinations of methylated and unmethylated sites, a phenomenon we have termed “intercellular mosaic methylation” (IMM, FIG. 25 and FIG. 4). Thus, the potential diversity of DNA methylation patterns in IMM across cells scales logarithmically with the number of motif sites targeted by the MTase exhibiting IMM.

FIG. 25A-B: Intercellular mosaic methylation (IMM) is distinct from other forms of mosaic-like DNA methylation. Conceptual illustration contrasting DNA methylome diversification and epigenetic inheritance between IMM and other mosaic-like mechanisms of heterogeneous DNA adenine methylation. (FIG. 25A) Cartoon illustrating the nature of methylomic diversity depict individual cells' chromosomes (gray bars) with methylation motifs (ovals). Oval colors represent distinct DNA methyltransferases (MTases). *Practically infinite, estimated as 2^{1,978} (there are roughly 1,978 MamA motif sites per replisome) under the assumption that methylation propensity on the daughter strand is independent from methylation status of other motif sites on the daughter strand and parent strand. **Assumes there are two phases. Some phase-variable MTases with more than two phases have been described. In these cases, potential states would be equivalent to the product of the sequence of numbers of phases for all independent phase-variable MTases. ***Calculated by Furuta and Kobayashi as the product of 1,000 DNA sequence specificities per MTase across 5 MTases in Helicobacter pylori (Furuta and Kobayashi, 2012). (FIG. 25B) Diagram illustrating the relationship between daughter and parent strains as it relates to conservation of the whole methylome (top) and at a single methylation site (bottom). Under the assumption of genuine stochasticity, IMM would practically never re-pattern the daughter strand identically to its parent. In contrast, the methylation status at any given methylation site would match between parent and daughter cells in 50% of cases.

This is not the first report of mosaic-like patterning of DNA adenine methylation in prokaryotes. Mosaicism can result from independent ON/OFF switching of multiple phase-variable MTases (Atack et al., 2018) or from domain movement of the target recognition domain (TRD) (Furuta and Kobayashi, 2012), a phenomenon known as “DoMo” (Furuta et al., 2014). However, IMM departs from these two previously described types of mosaic-like methylation heterogeneity in two important respects. First, in the degree of methylomic diversity it generates (FIG. 25A). Just as independent state-changes of multiple modification enzymes (Casadesus and Low, 2013) increases the diversity of epigenetic bacterial lineages beyond that of individual phase variation systems, IMM extends this diversity further still, scaling logarithmically with the number of motif sites targeted by the stochastic MTase. In nature, the set of methylation states that manifest may be constrained below this theoretical set by a variety of mechanisms, such as interaction with DNA binding proteins, or switch-like behavior between proximal MTase sites (Casadesus and Low, 2013). Nonetheless, the number of adoptable states is large enough that states are practically certain to differ between parent and daughter cells. Second, IMM is distinct in the pattern of epigenetic inheritance from parent to daughter cell (FIG. 25B). In mosaic-like methylomes driven by independent switching of phase-variable MTases (canonically a frameshift) and DoMo (Furuta et al., 2014) (via homologous recombining of TRDs) the methylome-patterning determinant is passed genetically to the daughter strand, unless there is a phase change (Sanchez-Romero and Casadesús, 2020). IMM lacks a genetic basis for transgenerational methylome inheritance and no epigenetic mechanism of inheritance for IMM in M. tuberculosis is known at present. Consequently, our current knowledge suggests a greater degree of methylomic diversity is spread throughout the population in each replication event, but that any advantageous methylation patterns would lack a stabilizing mechanism.

Throughout this manuscript we have referred to the DNA adenine methyltransferase encoded by Rv2756c as HsdM (hsdM for the gene) and its specificity subunit encoded by Rv2761, as HsdS (hsdS for the gene) to be consistent with previous work (Shell et al., 2013). It appears that Rv2756c was originally referred to as HsdM based on homology to hsdM in R-M systems—before the existence of its restriction component had been investigated—and has propagated through subsequent studies (Chiner-Oms et al., 2019; Gomez-Gonzalez et al., 2019; Phelan et al., 2018; Zhu et al., 2015). However, it has since been determined that Rv2756c lacks a functional HsdR component (Zhu et al., 2015). According to the prevailing nomenclature conventions, the symbol “hsd” is for Type 1 R-M systems (Loenen et al., 2014; Roberts et al., 2003), which Rv2756c is not part of, since it lacks a functional restriction component. Therefore, we propose that the orphan methyltransferase encoded by Rv2756c be renamed to MamC (mamC for the gene) Mycobacterial Adenine Methyltransferase C (since MamA and MamB are assigned to other mycobacterial DNA adenine methyltransferases). Likewise, we propose that the specificity subunit of MamC encoded by Rv2761 be renamed to mamS/MamS (formerly hsdS/HsdS) and the specificity subunit fragment encoded by Rv2755 (formerly hsdS.1/HsdS.1) to mamS.1/MamS.1. This proposed nomenclature retains the S and S.1 from hsdS and hsdS.1, is consistent with the extant naming convention of MamA and MamB, and removes the erroneous implication that HsdM/HsdS/HsdS.1 are part of a Type 1 R-M system.

In summary, HsdM is also named MamC, and subsequent literature may use MamC; and MamC (formerly HsdM) also requires its specificity subunit, a separately encoded protein, MamS (formerly HsdS); so in alternative embodiments, when HsdM/MamC is referred to, the whole functional complex of MamC and MamS is meant to be referred to.

Analysis of the relationship between methylation status of the conserved hypervariable sites (FIG. 20 and/or hypervariable among FIG. 21) and resistance phenotype demonstrates diagnostic potential for several of these sites for multiple drugs, as methylated fraction across reads associates strongly with resistance (FIG. 27A). FIG. 27B demonstrates that epigenetic information at two loci can discriminate resistance for the antitubercular drug isoniazid for multiple resistance-conferring mutations.

FIG. 27A illustrates the association between estimated methylated fraction (scaled IPD ratio) and resistance phenotypes. Eight anti-TB drugs and XDR vs. non-XDR as a binary phenotype were evaluated. Calculated among isolates with active MTase at hypervariable motif sites across 97 M. tuberculosis clinical isolates. Points above dashed line are motif sites whose methylated fraction correlated significantly with resistance phenotype (p<0.01, Benjamini-Hochberg).

FIG. 27B illustrates INH resistance conferred by different genotypic mechanisms clustered by methylation level at two motif sites.

### REFERENCES EXAMPLE 1

1. WHO. Global Tuberculosis Report 2017. (2017). doi:WHO/HTM/TB/2017.23
2. Cohen, K. A. et al. Evolution of Extensively Drug-Resistant Tuberculosis over Four Decades: Whole Genome Sequencing and Dating Analysis of Mycobacterium tuberculosis Isolates from KwaZulu-Natal. PLoS Med. 12, 1-22 (2015).
3. Zhu, L. et al. Precision methylome characterization of Mycobacterium tuberculosis complex (MTBC) using PacBio single-molecule real-time (SMRT) technology. Nucleic Acids Res. 44, 730-743 (2016).
4. Phelan, J. et al. Methylation in Mycobacterium tuberculosis is lineage specific with associated mutations present globally. Sci. Rep. 8, 160 (2018).
5. Low, D. A. & Casadesús, J. Clocks and switches: bacterial gene regulation by DNA adenine methylation. Curr. Opin. Microbiol. 11, 106-112 (2008).
6. Ardissone, S. et al. Cell Cycle Constraints and Environmental Control of Local DNA Hypomethylation in α-Proteobacteria. PLOS Genet. 12, e1006499 (2016).
7. Shell, S. S. et al. DNA Methylation Impacts Gene Expression and Ensures Hypoxic Survival of Mycobacterium tuberculosis. PLOS Pathog 9, e1003419 (2013).
8. Hernday, A., Krabbe, M., Braaten, B. & Low, D. Self-perpetuating epigenetic pili switches in bacteria. Proc. Natl. Acad. Sci. 99, 16470-16476 (2002).
9. Stephenson, S. A.-M. & Brown, P. D. Epigenetic Influence of Dam Methylation on Gene Expression and Attachment in Uropathogenic Escherichia coli. Front. public Heal. 4, 131 (2016).
10. Beaulaurier, J., Schadt, E. E. & Fang, G. Deciphering bacterial epigenomes using modern sequencing technologies. Nat. Rev. Genet. 1 (2018). doi:10.1038/s41576-018-0081-3
11. Gomez-Gonzalez, P. J. et al. An integrated whole genome analysis of Mycobacterium tuberculosis reveals insights into relationship between its genome, transcriptome and methylome. Sci. Rep. 9, 5204 (2019).
12. Casadesus, J. & Low, D. A. Programmed Heterogeneity: Epigenetic Mechanisms in Bacteria. J. Biol. Chem. 288, 13929-13935 (2013).
13. Wallecha, A., Munster, V., Correnti, J., Chan, T. & Woude, M. van der. Dam- and OxyR-Dependent Phase Variation of agn43: Essential Elements and Evidence for a New Role of DNA Methylation. J. Bacteriol. 184, 3338-3347 (2002).
14. Phasevarions of Bacterial Pathogens: Methylomics Sheds New Light on Old

Enemies. Trends Microbiol. 26, 715-726 (2018).
15. Beaulaurier, J. et al. Single molecule-level detection and long read-based phasing of epigenetic variations in bacterial methylomes. Nat Commun 6, (2015).
16. Zhu, L. et al. Precision methylome characterization of Mycobacterium tuberculosis complex (MTBC) using PacBio single-molecule real-time (SMRT) technology. Available at: http://nar.oxfordjournals.org. (Accessed: 18 Apr. 2016)
17. Pacific Biosciences. Kinetics Tools.
18. Otto, T. D., Dillon, G. P., Degrave, W. S. & Berriman, M. RATT: Rapid Annotation Transfer Tool. Nucleic Acids Res. 39, e57-e57 (2011).
19. Minch, K. J. et al. The DNA-binding network of Mycobacterium tuberculosis. Nat. Commun. 6, 5829 (2015).
20. Chiner-Oms, A., Gonzalez-Candelas, F. & Comas, I. Gene expression models based on a reference laboratory strain are poor predictors of Mycobacterium tuberculosis complex transcriptional diversity. Sci. Rep. 8, 3813 (2018).
21. Berney, M. et al. Essential roles of methionine and S-adenosylmethionine in the autarkic lifestyle of Mycobacterium tuberculosis. Proc. Natl. Acad. Sci. U.S.A. 112, 10008-10013 (2015).
22. Blow, M. J. et al. The Epigenomic Landscape of Prokaryotes. PLOS Genet 12, e1005854 (2016).
23. Otto, T. D., Dillon, G. P., Degrave, W. S. & Berriman, M. RATT: Rapid Annotation Transfer Tool. Nucleic Acids Res. 39, e57-e57 (2011).
24. Shell, S. S. et al. Leaderless Transcripts and Small Proteins Are Common Features of the Mycobacterial Translational Landscape. PLoS Genet. 11, (2015).
25. Cortes, T. et al. Genome-wide Mapping of Transcriptional Start Sites Defines an Extensive Leaderless Transcriptome in Mycobacterium tuberculosis. Cell Rep. 5, 1121-1131 (2013).
26. Chauhan, R. et al. Reconstruction and topological characterization of the sigma factor regulatory network of Mycobacterium tuberculosis. Nat. Commun. 7, 11062 (2016).
27. Staroń, A. et al. The third pillar of bacterial signal transduction: classification of the extracytoplasmic function (ECF) σ factor protein family. Mol. Microbiol. 74, 557-581 (2009).
28. Cook, G. M. et al. Physiology of Mycobacteria. Adv. Microb. Physiol. 55, 81-319 (2009).
29. Feklistov, A. & Darst, S. A. Structural basis for promoter-10 element recognition by the bacterial RNA polymerase a subunit. Cell 147, 1257-69 (2011).
30. Lee, W., VanderVen, B. C., Fahey, R. J. & Russell, D. G. Intracellular Mycobacterium tuberculosis exploits host-derived fatty acids to limit metabolic stress. J. Biol. Chem. 288, 6788-800 (2013).
31. Micklinghoff, J. C. et al. Role of the Transcriptional Regulator RamB (Rv0465c) in the Control of the Glyoxylate Cycle in Mycobacterium tuberculosis. J. Bacteriol. 191, (2009).
32. Murima, P. et al. A rheostat mechanism governs the bifurcation of carbon flux in mycobacteria. Nat. Commun. 7, 12527 (2016).
33. Nandakumar, M., Nathan, C. & Rhee, K. Y. Isocitrate lyase mediates broad antibiotic tolerance in Mycobacterium tuberculosis. Nat. Commun. 5, 4306 (2014).
34. Sirakova, T. D. et al. Identification of a diacylglycerol acyltransferase gene involved in accumulation of triacylglycerol in Mycobacterium tuberculosis under stress. Microbiology 152, 2717-2725 (2006).
35. Baek, S.-H., Li, A. H., Sassetti, C. M., Mitchell, M. & Milgram, E. Metabolic Regulation of Mycobacterial Growth and Antibiotic Sensitivity. PLoS Biol. 9, e1001065 (2011).
36. Baek, S.-H., Li, A. H., Sassetti, C. M., Mitchell, M. & Milgram, E. Metabolic Regulation of Mycobacterial Growth and Antibiotic Sensitivity. PLoS Biol. 9, e1001065 (2011).
37. Daniel, J., Maamar, H., Deb, C., Sirakova, T. D. & Kolattukudy, P. E. Mycobacterium tuberculosis Uses Host Triacylglycerol to Accumulate Lipid Droplets and Acquires a Dormancy-Like Phenotype in Lipid-Loaded Macrophages. PLoS Pathog. 7, e1002093 (2011).
38. Tong, J. et al. The FBPase Encoding Gene glpX Is Required for Gluconeogenesis, Bacterial Proliferation and Division In Vivo of Mycobacterium marinum. PLoS One 11, e0156663 (2016).
39. Gago, G., Kurth, D., Diacovich, L., Tsai, S.-C. & Gramajo, H. Biochemical and Structural Characterization of an Essential Acyl Coenzyme A Carboxylase from Mycobacterium tuberculosis. J. Bacteriol. 188, (2006).
40. Cronan, J. E. & Lin, S. Synthesis of the α,ω-dicarboxylic acid precursor of biotin by the canonical fatty acid biosynthetic pathway. Curr. Opin. Chem. Biol. 15, 407-413 (2011).
41. Gopinath, K., Moosa, A., Mizrahi, V. & Warner, D. F. Vitamin B₁₂ metabolism in Mycobacterium tuberculosis. Future Microbiol. 8, 1405-1418 (2013).
42. Minnikin, D. E., Kremer, L., Dover, L. G. & Besra, G. S. The Methyl-Branched Fortifications of Mycobacterium tuberculosis. Chem. Biol. 9, 545-553 (2002).
43. Constant, P. et al. Role of the pks15/1 gene in the biosynthesis of phenolglycolipids in the Mycobacterium tuberculosis complex. Evidence that all strains synthesize glycosylated p-hydroxybenzoic methyl esters and that strains devoid of phenolglycolipids harbor a frameshift mutation in the pks15/1 gene. J. Biol. Chem. 277, 38148-58 (2002).
44. Caws, M. et al. The Influence of Host and Bacterial Genotype on the Development of Disseminated Disease with Mycobacterium tuberculosis. PLoS Pathog. 4, e1000034 (2008).
45. Balabanova, Y. et al. Beijing clades of Mycobacterium tuberculosis are associated with differential survival in HIV-negative Russian patients. Infect. Genet. Evol. 36, 517-523 (2015).
46. Mishra, A. K. et al. Identification of an ?(1?6) mannopyranosyltransferase (MptA), involved in Corynebacterium glutamicum lipomanann biosynthesis, and identification of its orthologue in Mycobacterium tuberculosis. Mol. Microbiol. 65, 1503-1517 (2007).
47. Scherman, H. et al. Identification of a Polyprenylphosphomannosyl Synthase Involved in the Synthesis of Mycobacterial Mannosides. J. Bacteriol. 191, (2009).
48. De Smet, K. A. L., Brown, I. N., Weston, A., Young, D. B. & Robertson, B. D. Three pathways for trehalose biosynthesis in mycobacteria. Microbiology 146, 199-208 (2000).
49. Anthony Malinga, L., Stoltz, A. & Walt, M. van der. Efflux Pump Mediated Second-Line Tuberculosis Drug Resistance. Mycobact. Dis. 6, 1-9 (2016).
50. Morris, R. P. et al. Ancestral antibiotic resistance in Mycobacterium tuberculosis. Proc. Natl. Acad. Sci. 102, 12200-12205 (2005).
51. Fisher, R. A., Gollan, B. & Helaine, S. Persistent bacterial infections and persister cells. Nat. Rev. Microbiol. 15, 453-464 (2017).
52. Wagner, D. et al. Elemental Analysis of Mycobacterium avium-, Mycobacterium tuberculosis-, and Mycobacterium smegmatis-Containing Phagosomes Indicates Pathogen-Induced Microenvironments within the Host Cell's Endosomal System. J. Immunol. 174, 1491-1500 (2005).
53. Kurthkoti, K. et al. The Capacity of Mycobacterium tuberculosis To Survive Iron Starvation Might Enable It To Persist in Iron-Deprived Microenvironments of Human Granulomas. MBio 8, e01092-17 (2017).
54. Darwin, K. H. Mycobacterium tuberculosis and Copper: A Newly Appreciated Defense against an Old Foe? J. Biol. Chem. 290, 18962-6 (2015).
55. Brown, K. A. & Ratledge, C. THE EFFECT OF p-AMINOSALICYCLIC ACID ON IRON TRANSPORT AND ASSIMILATION IN MYCOBACTERIA. Biochimica et Biophysica Acta 385, (1975).
56. Raghu, B., Raghupati Sarma, G. & Venkatesan, P. Effect of Anti-tuberculosis Drugs on the Iron-Sequestration Mechanisms of Mycobacteria.
57. Peterson, E. J. R. R. et al. A high-resolution network model for global gene regulation in Mycobacterium tuberculosis. Nucleic Acids Res. 42, gku777 (2014).
58. Gago, G., Kurth, D., Diacovich, L., Tsai, S.-C. & Gramajo, H. Biochemical and Structural Characterization of an Essential Acyl Coenzyme A Carboxylase from Mycobacterium tuberculosis. J. Bacteriol. 188, (2006).
59. Cronan, J. E. & Lin, S. Synthesis of the α,ω-dicarboxylic acid precursor of biotin by the canonical fatty acid biosynthetic pathway. Curr. Opin. Chem. Biol. 15, 407-413 (2011).
60. Lee, J. J. et al. Glutamate mediated metabolic neutralization mitigates propionate toxicity in intracellular Mycobacterium tuberculosis. Sci. Rep. 8, 8506 (2018).
61. Wipperman, M. F., Yang, M., Thomas, S. T. & Sampson, N. S. Shrinking the FadE Proteome of Mycobacterium tuberculosis: Insights into Cholesterol Metabolism through Identification of an α2β2 Heterotetrameric Acyl Coenzyme A Dehydrogenase Family. J. Bacteriol. 195, (2013).
62. Domenech, P., Reed, M. B., Barry, C. E. & III. Contribution of the Mycobacterium tuberculosis MmpL protein family to virulence and drug resistance. Infect. Immun. 73, 3492-501 (2005).
63. Turapov, O. et al. Oleoyl Coenzyme A Regulates Interaction of Transcriptional Regulator RaaS (Rv1219c) with DNA in Mycobacteria. J. Biol. Chem. 289, 25241-25249 (2014).
64. Mustyala, K. K., Malkhed, V., Chittireddy, V. R. R. & Vuruputuri, U. Identification of Small Molecular Inhibitors for Efflux Protein: DrrA of Mycobacterium tuberculosis. Cell. Mol. Bioeng. 9, 190-202 (2016).
65. Colangeli, R. et al. The Mycobacterium tuberculosis iniA gene is essential for activity of an efflux pump that confers drug tolerance to both isoniazid and ethambutol. Mol. Microbiol. 55, 1829-1840 (2005).
66. Gupta, A. K. et al. Microarray Analysis of Efflux Pump Genes in Multidrug-Resistant Mycobacterium tuberculosis During Stress Induced by Common Anti-Tuberculous Drugs. Microb. Drug Resist. 16, 21-28 (2010).
67. Duan, W. et al. Mycobacterium tuberculosis Rv1473 is a novel macrolides ABC Efflux Pump regulated by WhiB7. Future Microbiol. 14, 47-59 (2019).
68. Parida, S. K. et al. Totally drug-resistant tuberculosis and adjunct therapies. J. Intern. Med. 277, 388-405 (2015).
69. Nieto R, L. M. et al. Biochemical characterization of isoniazid resistant Mycobacterium tuberculosis: can the analysis of clonal strains reveal novel targetable pathways? Mol. Cell. Proteomics (2018).
70. Nosova, E. Y. et al. Analysis of mutations in the gyrA and gyrB genes and their association with the resistance of Mycobacterium tuberculosis to levofloxacin, moxifloxacin and gatifloxacin. J. Med. Microbiol. 62, 108-113 (2013).
71. Schuessler, D. L. et al. Induced ectopic expression of HigB toxin in Mycobacterium tuberculosis results in growth inhibition, reduced abundance of a subset of mRNAs and cleavage of tmRNA. Mol. Microbiol. 90, n/a-n/a (2013).
72. Chownk, M., Kaur, J., Singh, K. & Kaur, J. mbtJ: an iron stress-induced acetyl hydrolase/esterase of Mycobacterium tuberculosis helps bacteria to survive during iron stress. Future Microbiol. 13, 547-564 (2018).
73. Game of Somes: Protein Destruction for Mycobacterium tuberculosis Pathogenesis. Trends Microbiol. 24, 26-34 (2016).
74. Wang, K. et al. The Expression of ABC Efflux Pump, Rv1217c-Rv1218c, and Its Association with Multidrug Resistance of Mycobacterium tuberculosis in China. Curr. Microbiol. 66, 222-226 (2013).
75. De Keijzer, J. et al. Mechanisms of Phenotypic Rifampicin Tolerance in Mycobacterium tuberculosis Beijing Genotype Strain B0/W148 Revealed by Proteomics. J. Proteome Res. 15, 1194-1204 (2016).
76. Reed, M. B., Gagneux, S., DeRiemer, K., Small, P. M. & Barry, C. E. The W-Beijing lineage of Mycobacterium tuberculosis overproduces triglycerides and has the DosR dormancy regulon constitutively upregulated. J. Bacteriol. 189, 2583-2589 (2007).
77. Huet, G. et al. A lipid profile typifies the Beijing strains of Mycobacterium tuberculosis: identification of a mutation responsible for a modification of the structures of phthiocerol dimycocerosates and phenolic glycolipids. J. Biol. Chem. 284, 27101-13 (2009).
78. Cortes, T. et al. Delayed effects of transcriptional responses in Mycobacterium tuberculosis exposed to nitric oxide suggest other mechanisms involved in survival. Sci. Rep. 7, 8208 (2017).
79. Vilchèze, C. et al. Enhanced respiration prevents drug tolerance and drug resistance in Mycobacterium tuberculosis. Proc. Natl. Acad. Sci. 114, 4495-4500 (2017).
80. Keren, I., Minami, S., Rubin, E. & Lewis, K. Characterization and Transcriptome Analysis of Mycobacterium tuberculosis Persisters. MBio 2, (2011).
81. Bussi, C. & Gutierrez, M. G. Mycobacterium tuberculosis infection of host cells in space and time. FEMS Microbiol. Rev. (2019). doi:10.1093/femsre/fuz006
82. Browning, D. F. & Busby, S. J. W. Local and global regulation of transcription initiation in bacteria. Nat. Rev. Microbiol. 14, 638-650 (2016).
83. Gries, T. J., Kontur, W. S., Capp, M. W., Saecker, R. M. & Record, M. T. One-step DNA melting in the RNA polymerase cleft opens the initiation bubble to form an unstable open complex. Proc. Natl. Acad. Sci. 107, 10418-10423 (2010).
84. Saecker, R. M. et al. Kinetic Studies and Structural Models of the Association of E. coli σ70 RNA Polymerase with the XPR Promoter: Large Scale Conformational Changes in Forming the Kinetically Significant Intermediates. J. Mol. Biol. 319, 649-671 (2002).
85. Krokan, H. E. & Bjørås, M. Base excision repair. Cold Spring Harb. Perspect. Biol. 5, a012583 (2013).
86. Campbell, J. L. & Kleckner, N. E. coli oriC and the dnaA gene promoter are sequestered from dam methyltransferase following the passage of the chromosomal replication fork. Cell 62, 967-979 (1990).
87. Ardissone, S. et al. Cell Cycle Constraints and Environmental Control of Local DNA Hypomethylation in α-Proteobacteria. PLoS Genet. 12, e1006499 (2016).
88. Adhikari, S. & Curtis, P. D. DNA methyltransferases and epigenetic regulation in bacteria. FEMS Microbiol. Rev. fuw023 (2016). doi:10.1093/femsre/fuw023
89. Elghraoui, A., Modlin, S. J. & Valafar, F. SMRT genome assembly corrects reference errors, resolving the genetic basis of virulence in Mycobacterium tuberculosis. BMC Genomics 18, 302 (2017).
90. Chin, C.-S. et al. Nonhybrid, finished microbial genome assemblies from long-read SMRT sequencing data. Nat. Methods 10, 563-569 (2013).
91. Koren, S. et al. Canu: scalable and accurate long-read assembly via adaptive k-mer weighting and repeat separation. Genome Res. 27, 722-736 (2017).
92. Hunt, M. et al. Circlator: automated circularization of genome assemblies using long sequencing reads. Genome Biol. 16, 294 (2015).
93. Chaisson, M. J. & Tesler, G. Mapping single molecule sequencing reads using Basic Local Alignment with Successive Refinement (BLASR): Theory and Application. BMC Bioinformatics 13, 238 (2012).
94. English, A. C., Salerno, W. J. & Reid, J. G. PBHoney: identifying genomic variants via long-read discordance and interrupted mapping. BMC Bioinformatics 15, 180 (2014).
95. Shabbeer, A. et al. TB-Lineage: An online tool for classification and analysis of strains of Mycobacterium tuberculosis complex. Infect. Genet. Evol. 12, 789-797 (2012).
96. Lew, J. M., Kapopoulou, A., Jones, L. M. & Cole, S. T. TubercuList—10 years after. Tuberculosis (Edinb). 91, 1-7 (2011).
97. Seemann, T. Prokka: rapid prokaryotic genome annotation. Bioinformatics 30, 2068-2069 (2014).
98. Powell, S. et al. eggNOG v3.0: Orthologous groups covering 1133 organisms at 41 different taxonomic ranges. Nucleic Acids Res. 40, 284-289 (2012).
99. Hyatt, D. et al. Prodigal: Prokaryotic gene recognition and translation initiation site identification. BMC Bioinformatics 11, (2010).
100. Katoh, K. & Standley, D. M. MAFFT multiple sequence alignment software version 7: improvements in performance and usability. Mol. Biol. Evol. 30, 772-80 (2013).
101. Marcais, G. et al. MUMmer4: A fast and versatile genome alignment system. PLOS Comput. Biol. 14, e1005944 (2018).
102. McLaren, W. et al. The Ensembl Variant Effect Predictor. Genome Biol. 17, 122 (2016).

103. Stamatakis, A. RAxML Version 8: A tool for Phylogenetic Analysis and Post-Analysis of Large Phylogenies. Bioinformatics 30, btu033-btu033 (2014). 104. Letunic, I. & Bork, P. Interactive tree of life (iTOL) v3: an online tool for the display and annotation of phylogenetic and other trees. Nucleic Acids Res. 44, W242-5 (2016).
105. Grant, C. E., Bailey, T. L. & Noble, W. S. FIMO: scanning for occurrences of a given motif. Bioinformatics 27, 1017-1018 (2011).

### REFERENCES EXAMPLE 2

1. Beaulaurier, J. et al. Single molecule-level detection and long read-based phasing of epigenetic variations in bacterial methylomes. Nat Commun 6, 7438 (2015).
2. Atack, J. M., Tan, A., Bakaletz, L. O., Jennings, M. P. & Seib, K. L. Phasevarions of Bacterial Pathogens: Methylomics Sheds New Light on Old Enemies. Trends Microbiol. 26, 715-726 (2018).
3. Furuta, Y. & Kobayashi, I. Mobility of DNA sequence recognition domains in DNA methyltransferases suggests epigenetics-driven adaptive evolution. Mob. Genet. Elements 2, 292-296 (2012).
4. Furuta, Y. et al. Methylome Diversification through Changes in DNA Methyltransferase Sequence Specificity. PLOS Genet 10, e1004272 (2014).
5. Casadesus, J. & Low, D. A. Programmed Heterogeneity: Epigenetic Mechanisms in Bacteria. J. Biol. Chem. 288, 13929-13935 (2013).
6. Sanchez-Romero, M. A. & Casadesús, J. The bacterial epigenome. Nature Reviews Microbiology 18, 7-20 (2020).
7. Shell, S. S. et al. DNA methylation impacts gene expression and ensures hypoxic survival of Mycobacterium tuberculosis. PLoS Pathog. 9, e1003419 (2013).
8. Gomez-Gonzalez, P. J. et al. An integrated whole genome analysis of Mycobacterium tuberculosis reveals insights into relationship between its genome, transcriptome and methylome. Sci. Rep. 9, 5204 (2019).
9. Chiner-Oms, A. et al. Genome-wide mutational biases fuel transcriptional diversity in the Mycobacterium tuberculosis complex. Nat. Commun. 10, 3994 (2019).
10. Zhu, L. et al. Precision methylome characterization of Mycobacterium tuberculosis complex (MTBC) using PacBio single-molecule real-time (SMRT) technology. Nucleic Acids Res. 44, gkv1498 (2015).
11. Phelan, J. et al. Methylation in Mycobacterium tuberculosis is lineage specific with associated mutations present globally. Sci. Rep. 8, 160 (2018).
12. Loenen, W. A. M., Dryden, D. T. F., Raleigh, E. A. & Wilson, G. G. Type I restriction enzymes and their relatives. Nucleic Acids Res. 42, 20-44 (2014).
13. Roberts, R. J. et al. A nomenclature for restriction enzymes, DNA methyltransferases, homing endonucleases and their genes. Nucleic Acids Research 31, 1805-1812 (2003).

A number of embodiments of the invention have been described. Nevertheless, it can be understood that various modifications may be made without departing from the spirit and scope of the invention. Accordingly, other embodiments are within the scope of the following claims.

1: A method for treating or ameliorating a Mycobacterium tuberculosis (TB) or a Mycobacterium africanum infection, comprising inhibiting DNA methylation in an infecting Mycobacterium tuberculosis (TB) or a Mycobacterium africanum bacterium or bacterial population, the method comprising administering to an individual in need thereof a DNA methylation inhibitory molecule capable of inhibiting a Mycobacterium tuberculosis or a Mycobacterium africanum DNA methyltransferase,
wherein optionally the DNA methylation inhibitory molecule is formulated as a pharmaceutical composition, or is formulated for administration in vivo; or formulated for enteral or parenteral administration, or for oral, intravenous (IV) or intrathecal (IT) administration, wherein optionally the compound or formulation is administered orally, parenterally, by inhalation spray, nasally, topically, intrathecally, intrathecally, intracerebrally, epidurally, intracranially or rectally,
and optionally the DNA methylation inhibitory molecule or the formulation or pharmaceutical composition is contained in or carried in a nanoparticle, a particle, a micelle or a liposome or lipoplex, a polymersome, a polyplex or a dendrimer,
and optionally the DNA methylation inhibitory molecule, or the formulation or pharmaceutical composition, is formulated as, or contained in, a nanoparticle, a liposome, a tablet, a pill, a capsule, a gel, a geltab, a liquid, a powder, an emulsion, a lotion, an aerosol, a spray, a lozenge, an aqueous or a sterile or an injectable solution, or an implant,
and optionally the DNA methylation inhibitory molecule is an inhibitory nucleic acid, the optionally the inhibitory nucleic acid is contained in a nucleic acid construct or a chimeric or a recombinant nucleic acid, or an expression cassette, vector, plasmid, phagemid or artificial chromosome, optionally stably integrated into a TB cell's chromosome, or optionally stably episomally expressed in a TB cell,
and optionally the inhibitory nucleic acid is or comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), or a ribozyme.

2: The method of claim 1, wherein the Mycobacterium tuberculosis or the Mycobacterium africanum DNA methyltransferase is a methyltransferase selected from the group consisting of MamA, MamB and HsdM.

3: The method of claim 1, wherein the DNA methylation inhibitory molecule capable of inhibiting a Mycobacterium tuberculosis or Mycobacterium africanum DNA methyltransferase is or comprises a small molecule, an inhibitory nucleic acid (optionally and miRNA or antisense molecule), polypeptide or peptide (optionally an antibody capable of specifically binding to the Mycobacterium tuberculosis or Mycobacterium africanum DNA methyltransferase and inhibiting its expression or activity, a lipid or a polysaccharide.

4: A kit for or treating or ameliorating a tuberculosis (TB) infection, wherein optionally Mycobacterium tuberculosis (TB) or Mycobacterium africanum is the microbacterial agent of infection, comprising a DNA methylation inhibitory molecule capable of inhibiting a Mycobacterium tuberculosis or Mycobacterium africanum DNA methyltransferase,
wherein optionally the DNA methylation inhibitory molecule is or comprises a DNA methylation inhibitory molecule used to practice a method of claim 1,
and optionally the kit further comprises instructions for practicing a method of any of the preceding claims.

5: A method for treating or ameliorating a tuberculosis (TB) infection,
wherein optionally Mycobacterium tuberculosis (TB) or Mycobacterium africanum is the microbacterial agent of infection,
comprising inhibiting expression of at least one gene as set forth in Table 1 (FIG. 8), Table 2 (FIG. 9), the “TSS” column of FIG. 20, and/or in the first column (labeled TSS) of FIG. 23,
the method comprising administering to an individual in need thereof a molecule capable of inhibiting expression of the gene or a polypeptide encoded by the gene.

6: The method of claim 5, wherein the molecule capable of inhibiting expression of the gene or a polypeptide encoded by the gene is or comprises a small molecule, an inhibitory nucleic acid (optionally and miRNA or antisense molecule), polypeptide or peptide (optionally an antibody capable of specifically binding to the Mycobacterium tuberculosis or the Mycobacterium africanum DNA methyltransferase and inhibiting its expression or activity, a lipid or a polysaccharide.

7: A kit for or treating or ameliorating a Mycobacterium tuberculosis (TB) or a Mycobacterium africanum infection, comprising a molecule capable of inhibiting expression of at least one gene as set forth in Table 1 (FIG. 8), Table 2 (FIG. 9), the “TSS” column of FIG. 20, and/or in the first column (labeled TSS) of FIG. 23, and further comprising instructions for practicing a method of claim 1.

8: A method for identifying targets for treating, ameliorating, diagnosing, or prognosing infection by a microbial agent, the method comprising an analysis of single-molecule sequencing data,
wherein the analysis comprises deducing knowledge of a DNA sequence and the boundaries of genetic elements encoded therein and deducing knowledge of the base modification status of bases comprising the deduced DNA sequence.

9: The method of claim 8, wherein the method provides evidence of druggability and/or utility to a user for helping to clear microbial infection, and the method further comprising a series of single-molecule sequencing data processing steps that incorporate signals of DNA sequence order and DNA sequence modification, such that their coincidence is inferred and coincidences between base modification and identified genetic elements of the sequence that evidence druggability and/or utility for helping to clear microbial infection are returned to the user.

10: The method of claim 8, wherein the genetic elements encoding a plurality of base modifying enzymes are deduced and/or prior knowledge of the identity of a plurality of genetic elements encoding base modifying enzymes are collated and correlated to sequencing kinetics of sequence contexts that are known/deduced to methylate, in order to deduce of the presence or absence of the phenomenon of intercellular mosaic methylation in the analyzed sample.

11: The method of claim 8, wherein the single-molecule sequencing data is processed through a series of analyses and returns estimates of the likelihood of prognostic outcomes based on the presence, absence, or contingencies dictating the presence/absence of the phenomenon of intercellular mosaic methylation to the user of the embodiment.

12-32. (canceled)
